(19) United States
(12) Patent Application Publication
(10) Pub. No.: US 2024/0197890 A1
(43) Pub. Date: Jun. 20, 2024

(54) FLAVONE DERIVATIVES AND USES IN TARGETING CK2-MEDIATED DISEASES AND CONDITIONS

(71) Applicant: Salk Institute for Biological Studies, La Jolla, CA (US)

(72) Inventors: Ioana I. Nitulescu, San Diego, CA (US); Fred H. GAGE, La Jolla, CA (US); James K. TUCKER, San Diego,, CA (US)

(21) Appl. No.: 17/990,489

(22) Filed: Nov. 18, 2022

Related U.S. Application Data
(63) Continuation of application No. PCT/US2021/032876, filed on May 18, 2021.
(60) Provisional application No. 63/062,869, filed on Aug. 7, 2020.
(60) Provisional application No. 63/026,647, filed on May 18, 2020.

(51) Int. Cl. A61K 47/55; A61P 25/28; A61P 29/00; A61P 31/14

(52) U.S. Cl. CPC A61K 47/55; A61P 25/28; A61P 29/00; A61P 31/14

(57) ABSTRACT

Flavone derivatives and pharmaceutical compositions including the derivatives are disclosed. In some instances, the compounds have increased aqueous solubility, bioavailability, and ability to cross the blood-brain-barrier. The compounds may be used to inhibit CK2 activity and/or to treat diseases and conditions mediated at least in part by CK2 enzyme.

### CROSS REFERENCE TO RELATED APPLICATIONS

This is the U.S. National Stage of International Application No. PCT/US2021/032876, filed May 18, 2021, which was published in English under PCT Article 21(2), which in turn claims the benefit of the earlier filing dates of U.S. Provisional Application No. 63/062,869, filed Aug. 7, 2020, and U.S. Provisional Application No. 63/026,647, filed May 18, 2020, each of which is incorporated by reference herein in its entirety.

### FIELD

This invention concerns embodiments of flavone derivatives and a method for using the derivatives in inhibiting CK2 enzyme activity, thereby treating CK2-mediated diseases and/or conditions.

### BACKGROUND

Neuroinflammation has emerged as a significant etiological factor for numerous neurological and psychiatric diseases, including Alzheimer's disease (AD), Huntington's disease (HD), and Parkinson's disease (PD). Microglia and astrocytes, the brain's resident immune cells, become chronically activated and perpetuate a destructive loop of pro-inflammatory cytokine release. Inflammation and mitochondrial dysfunction are two known processes that contribute to AD by influencing the pathogenesis and severity. Over time, chronic inflammation propagated by microglia and astrocytes leads to neurotoxicity and defects in normal brain function. Notably, inflammation might precede neuronal loss, the hallmark of many neurodegenerative diseases. In turn, mitochondrial dysfunction in neurons, which rely on oxidative phosphorylation for energy, leads to neuronal death. Chronic inflammation in AD is evidenced by the presence of reactive glia in postmortem studies and inflammatory biomarkers in serum and cerebrospinal fluid (CSF).

Casein kinase II (CK2) is a kinase with a brain-enriched variant that shows increased activity in inflammatory and neurodegenerative diseases. Remarkably, CK2 regulates mitochondrial homeostasis as well as innate immune pathways. In neurons, defects in mitophagy and mitochondrial fragmentation have been linked to degeneration in AD patients as well as AD mouse models. Protein kinase CK2 regulates both of these AD-associated processes. First, CK2 regulates innate immunity pathways, and CK2 inhibitors have shown efficacy in immune-driven cancers. CK2 protein levels increase in peripheral immune cells upon induction by pro-inflammatory stimuli such as LPS and TNFα, and CK2 phosphorylates important mediators of inflammation, including NF-κB, IκBa, and AKT. Second, CK2 regulates two aspects of mitochondrial homeostasis, mitophagy and mitochondrial fission. CK2 blocks mitophagy through phosphorylation and inactivation of FUNDC1, which leads to accumulation of damaged mitochondria and mitochondrial apoptosis. CK2 also indirectly upregulates phosphorylation of MFF, leading to mitochondrial fission. In models of vascular injury, CK2 knockdown and CK2 inhibitor treatment restores mitophagy and blocks mitochondrial fission.

Finally, CK2 is highly expressed in the brain and one of its catalytic subunits, CK2A2, is enriched in the brain relative to other tissues. There is evidence of dysregulation of CK2 at the gene and protein level in neurodegenerative diseases. For example, higher CK2 levels were observed in astrocytes from AD patients. In PD, CK2 regulatory subunits were reported to co-localize with Lewy bodies. Notably, CK2 overexpression causes cognitive decline in wild-type mice, and higher CK2 levels and/or activity was observed in transgenic mice for AD (APP/PS1 and 3×Tg models), PD (alpha-synuclein A53T model), and Huntington's disease (zQ175 model).

### SUMMARY

This disclosure concerns embodiments of flavone derivatives and methods for using the compounds. In some embodiments, the compound has a structure according to formula I, or a pharmaceutically acceptable salt, hydrate, stereoisomer, or tautomer thereof:

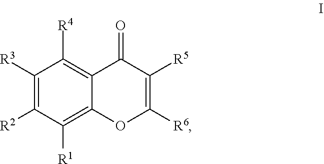

where R¹ is halo, H, substituted or unsubstituted aryl, substituted or unsubstituted heteroaryl, —SF₅, or -L-Q where L is a linker and Q is an E3-ligase binding moiety; R² is H, —O(CH₂)ₘR^{a} where m is an integer greater than ore equal to zero, -L-Q; R³ is H, —OR^{a},

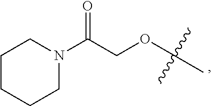 [SMILES: *OCC(=O)N1CCCCC1]

a hydrophobic group (e.g. aliphatic, substituted aliphatic (—CF₃), —SF₅), or -L-Q; R⁴ and R⁵ independently are H or —OR^{a}; R^{a} is H, substituted or unsubstituted aliphatic, substituted or unsubstituted heteroaliphatic, or acyl; and R⁶ is

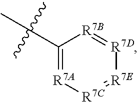

—(CH₂)ⁿ—R^{b}, —C≡C—R^{b}, substituted aliphatic, H, or halo, where n is an integer from 1-10 and R^{b} is substituted or unsubstituted heteroaliphatic or —OR^{a}, or R^{b} is R¹¹, where R¹¹ is substituted or unsubstituted heteroaryl, or substituted or unsubstituted aryl, where R^{7A} N or CH, R^{7B} is N or CH, R^{7C} is N or C—R⁸, R^{7D} is N or C—R¹⁰, and R^{7E} is C—R⁹ or N⁺—O⁻, and 0, 1, or 2 of R^{7A}—R^{7D} is N, R⁸ is —OR^{a}, H, substituted or unsubstituted alkyl, or halo, R⁹ is —OR^{a}, —CN, —C(O)OR^{a}, or azole, and R¹⁰ is H, alkynyl, -alkynyl-R¹¹, -alkynyl-heteroaliphatic, halo, —OR^{a}, or a hydrophobic group, or R⁸/R⁹ or R⁹/R¹⁰ together with the atoms to which they are bound form a substituted or unsubstituted heteroaliphatic or heteroaryl ring. In certain embodiments, if R^{7A} and R^{7B} are both CH, then (i) R³ is not alkyl, or (ii) R⁹ and R¹⁰ together with the atoms to which they are bound form a substituted or unsubstituted heteroaliphatic or heteroaryl ring including at least one N atom in the ring, or (iii) R¹ is furan substituted with —C(O)OR^{a}, or (iv) one of R^{7C} or R^{7D} is N, or (v) R^{7E} is N⁺—O⁻.

In some embodiments, the compound has a structure according to any one of formulas II or III, or a pharmaceutically acceptable salt, hydrate, stereoisomer, or tautomer thereof:

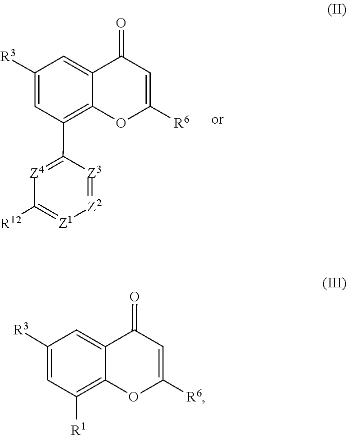

where each of Z¹-Z⁴ is CH or one of Z¹-Z⁴ is N and the others of Z¹-Z⁴ are CH; and R¹² is halo, alkynyl, or -alkynyl-R¹¹; R¹, R³, and R⁶ are as previously defined.

This disclosure also encompasses pharmaceutical compositions. A pharmaceutical composition includes a compound as disclosed herein and a pharmaceutically acceptable excipient. Methods of using compounds according to formulas I-IV to reduce or inhibit CK2 enzyme activity are disclosed. In some embodiments, the compound is a compound where R¹, R², or R³ is -L-Q, and inhibiting CK2 enzyme activity further comprises degrading the CK2 enzyme. In some embodiments, a method of inhibiting CK2 activity includes contacting a cell that expresses CK2 enzyme with an effective amount of one or more compounds as disclosed herein, thereby inhibiting activity of the CK2 enzyme. The cell may be an astrocyte, a microglia, a neuron, a white blood cell, an adipocyte, a myocyte, or an epithelial cell. In any of the foregoing embodiments, inhibiting activity of the CK2 enzyme may reduce or inhibit phosphorylation of one or more biomarkers, increase mitophagy, decrease mitochondrial fission, increase mitochondrial function, or any combination thereof. In some examples, the term “inhibition” does not require a complete elimination of activity. Thus, in some examples, “inhibition” can refer to a reduction in detectable CK2 activity, for example as indicated by reduced phosphorylation of one or more biomarkers, for example a reduction of at least 50%, at least 60%, at least 70%, at least 80%, at least 90%, at least 95%, at least 98%, at least 99%, or even 100%, for example as compared to such activity in the absence of a compound according to formulas I-IV.

In any of the foregoing or following embodiments, contacting the cell with the one or more compounds may comprise administering a therapeutically effective amount of the one or more compounds, or an amount of a pharmaceutical composition comprising the therapeutically effective amount of the one or more compounds, to a subject. In some embodiments, the subject has a disease or condition characterized at least in part by dysregulated CK2 enzyme activity. Administering the therapeutically effective amount of the one or more compounds or the amount of the pharmaceutical composition comprising the therapeutically effective amount of the one or more compounds to the subject may ameliorate at least one sign or symptom of the disease or condition.

In any of the foregoing embodiments, the disease or condition may be characterized at least in part by inflammation, e.g., neuroinflammation. In some embodiments, the disease or condition is cancer, cardiac hypertrophy, cystic fibrosis, a neurodegenerative disease (including, but not limited to, Parkinson's disease, Huntington's disease, Alzheimer's disease, multiple sclerosis, or amyotrophic lateral sclerosis), bipolar disorder, depression, a viral infection (including, but not limited to, coronavirus infections such as SARS-CoV-2 infections), obesity, diabetes mellitus, atherosclerosis, epilepsy, or any combination thereof.

The foregoing and other objects and features of the invention will become more apparent from the following detailed description, which proceeds with reference to the accompanying figures.

### BRIEF DESCRIPTION OF THE DRAWINGS

FIGS. 1A-1F demonstrate anti-inflammatory activity and unbiased target identification of flavones in glia. FIG. 1A shows that 20 μM apigenin (API) blocks IL6, IL8, MCP-1, and TNFα cytokine production in human primary astrocytes (HCA); representative flow cytometry traces from 12 independent experiments. FIG. 1B shows that chrysoeriol (CHR) suppresses LPS-induced pro-inflammatory cytokine release and boosts anti-inflammatory cytokine release in human iPSC-derived microglia (n=2, mean fold-change). FIG. 1C shows that 20 μM CHR blocks microglial phagocytosis of E. coli-FITC conjugated beads (5 h treatment, n=2-3, mean±SD, one-way ANOVA with Dunnett's post-hoc test; representative of two independent experiments. FIG. 1D shows that CHR exhibits dose-dependent anti-inflammatory activity in human astrocytes and microglia (n=3, mean±SD). FIG. 1E shows thermal shift proteome profiling (TPP) in human iPSC-derived astrocytes; arrows indicate proteins exhibiting significant reproducible thermal shifts after CHR treatment in IL1β-activated astrocytes. FIG. 1F shows that an isothermal dose response thermal shift assay validates CK2 as a target of CHR; each dot represents normalized band intensity (Western blot); representative of two independent experiments. *P<0.05, **P<0.01, ***P<0.001, ****P<0.0001.

FIGS. 2A-2E show additional characterization of flavones for anti-inflammatory activity and cytotoxicity in astrocytes (5 h treatment); FIG. 2A is a series of graphs showing activity of several flavones and flavanones in a flow-cytometry-based assay of cytokine production; each dot represents one biological sample from 2-5 independent experiments (n=2-12, mean±SD, one-way ANOVA with Bonferroni post-hoc test); bar charts for naringenin (NAR) and chrysin (CHR) in the IL6 graph represent one experiment; FIG. 2B shows that NAR does not block IL6, IL8 MCP-1, and TNFα cytokine production in human cerebellar astrocytes (HCA; FIG. 2C is a dose-response curve of IL6 expression in TMF or dTMF-treated astrocytes activated with IL1-β (n=3-6, mean±SD), representative of 3 independent experiments; FIG. 2D shows normalized expression (IL6/GAPDH) relative to DMSO from a ddPCR experiment showing no effect of dCHR on IL6 expression in IL1-β-activated astrocytes (n=1-2 biological samples, Poisson error of >15,000 droplets); FIG. 2E is a bar graph showing that flavones do not reduce cell viability of primary human astrocytes at 20 μM; each dot represents percentage of non-Zombie Violet stained cells in one biological sample from 2-5 independent experiments (n=2-10, mean±SD).

FIGS. 3A-3F illustrate stabilization of CK2 and CK2 interactors by flavones using thermal shift proteome profiling (TPP) and Western blotting (WB); FIG. 3A shows thermal shift assay (TSA)-WB isothermal dose response showing stabilization of CK2a1 by CHR; FIG. 3B is a GeneMania network showing protein-protein interactions between hits from FIG. 1E (red nodes-stabilized, blue nodes-destabilized); FIG. 3C is an immunoblot showing physical interaction of PTGR1 and CKa1 by co-immunoprecipitation; FIG. 3D shows TPP in human iPSC-derived astrocytes where red denotes proteins exhibiting significant and reproducible thermal shifts after TMF treatment in IL1-β-activated astrocytes; FIG. 3E is a GeneMania network showing protein-protein interactions between several hits (red nodes-stabilized, blue nodes-destabilized, gray nodes—not significant) and related proteins (white nodes); FIG. 3F shows TSA-WB (left) with TMF and dTMF, along with band intensity quantification (right).

FIGS. 4A-4C provide additional characterization of flavones for in vitro and in-cell CK2 inhibition; FIG. 4A shows that flavones inhibit CK2a1 in vitro (PhosphoSens kinase assay), API (active), CHR (active), TMF (active) vs. dTMF, dCHR, NAR (inactive) vs. chrysin, scutellarein/SCUT (medium active); FIG. 4B is a kinase capture assay showing that apigenin is an ATP-competitive inhibitor of CK2a1 and CK2a2 in H1-derived astrocyte whole lysates (graph shows band intensity normalizations to a 150 kD protein stained by Amido Black; lines represent nonlinear curve fits of dose-curve data; representative of two independent experiments); FIG. 4C is a NanoBRET assay showing dose-dependent CK2a2 target engagement in HEK293T cells (each point mean±sem, n=3; non-linear fit was used to calculate IC₅₀).

FIGS. 5A-5D show that inhibition of CK2 kinase activity reduces inflammation in glia. FIG. 5A is a summary of SAR data showing that apigenin (API), chrysoeriol (CHR), and trimethoxyflavone (TMF) are active, chrysin and scutellarein are intermediately active, and dTMF, dCHR, and naringenin (NAR) are inactive at 20 μM. FIG. 5B shows that structurally unrelated CK2 inhibitor CX4945 blocks IL1β-induced IL6 upregulation as well as TMF (mean±SD, n=2, one-way ANOVA with Tukey's post-hoc test; normalized to DMSO, data are representative of three independent experiments). FIG. 5C shows that knockdown of CK2a1 and CK2a2 significantly blocks IL1β-induced IL8 upregulation; box plot with mean values and 25-75% CI of 4-5 independent experiments, total n=4-8 biological replicates, unpaired t-test. FIG. 5D shows that overexpression of CK2 kinase-dead mutants significantly blocks IL1β-induced IL6 and IL8 upregulation; mean of n=3 independent experiments, each value normalized within experiments to CK2a2 WT; one-way ANOVA with Sidak's post-hoc test. *P<0.05, **P<0.01, ***P<0.001, ****P<0.0001.

FIGS. 6A-6C show CK2 genetic perturbation assays and NF-KB immunoprecipitation; FIG. 6A shows the knockdown efficiency of experiments shown in FIG. 5B (mean expression shown with each dot representing an experiment); FIG. 6B is a Western blot confirming expression of CK2a1-HA and CK2a2-HA; FIG. 6C is an immunoblot of NF-κB showing NF-κB 529 phosphorylation is reduced with CHR (2 h treatment).

FIGS. 7A-7G show that CK2 inhibition attenuates inflammatory biochemical and transcriptional programs via NF-κB; FIG. 7A is a Western blot showing that CK2 levels increase with inflammation; FIG. 7B is an immunoblot and accompanying quantification showing that IκB S32 levels are increased with inflammation and decreased with CK2 inhibition after 5 hours; FIG. 7C is a series of immunofluorescence images showing reduction of nuclear pNF-κB after 1 h of CK2 inhibitor treatment (mean±SD, n=2-4, one-way ANOVA, Fisher's test); FIG. 7D is a series of immunofluorescence images and a graph showing reduction of nuclear pNF-κB after 5 h of CK2 inhibitor treatment (mean±SD, n=4, one-way ANOVA, Fisher's test); FIG. 7E is an X2K interaction network showing top enriched kinase modules, intermediate proteins (not labeled), and their downstream TF targets in IL1-β-stimulated astrocytes (unrelated to CK2 in gray, red edges denote CK2 interactions); FIG. 7F shows that immune signatures are significantly enriched in genes downregulated in inflamed astrocytes treated with API; FIG. 7G shows that CK2 inhibition reduced expression of neurotoxic A1 genes and increased expression of neuroprotective A2 genes.

FIGS. 8A-8E evaluate RNA-sequences in IL1-β and API-treated astrocytes; FIG. 8A is a bar graph depicting p-values associated with enriched kinases in IL1-β-treated astrocytes compared to controls (kinase enrichment analysis, KEA); FIG. 8B is a bar graph depicting p-values associated with enriched TFs and other chromatin factors in IL1-β-treated astrocytes compared to controls (ChEA); FIG. 8C is a Venn diagram showing overlap of significantly DE genes up in IL1-β and down in IL1-β-treated cells; FIG. 8D is a Venn diagram showing overlap of significantly De genes down in IL1-β and up in API+IL-1-β-treated cells; FIG. 8E is a rainfall plot showing rank of CMap signatures by z-score in terms of similarity to API and IL1-β-treated HCAs.

FIG. 9 shows that SQSTM1/p62 was induced by IL1β and reduced by CK2 inhibitor treatment (Bort=bortezomib).

FIG. 10 shows that CK2 inhibitor CHR reduced AKT pS129 levels that are induced in primary astrocytes stimulated with IL1β for 5 hours.

FIGS. 11A and 11B show that CK2 inhibitors CX-4945 and CHR reduced AKT pS473 levels in primary astrocytes stimulated with IL1β for 5 hours (FIG. 1A); FIG. 11B shows the controls—total AKT levels in cells.

FIGS. 12A-12E show effects of CK2 in neurological diseases; FIG. 12A is a box plot showing that Ck2 enzymatic activity is higher in postmortem Parkinson's disease (PD) patient samples compared to controls (mean and 25-75% Cl, n=6 per group, two-tailed t-test); FIG. 12B shows expression of CK2 genes in postmortem brains (parietal cortex) from patients with dementia (Dem, n=21) or controls (Ct, n=27) from the Aging, Dementia, and TBI RNA-seq study, the box plot depicts the mean z-score (two-tailed t-test); FIG. 12C is representative immunoblots and quantification showing secreted HMGB1 and cellular CK2a1 and CK2a2 protein levels normalized to GAPDH in Alzheimer's disease (AD) vs. control iPSC-derived astrocytes (mean and whiskers showing min/max); FIG. 12D is a Western blot and quantification of HMGB1 secretion after CK2 inhibitor treatment; FIG. 12E is representative immunoblots and quantification showing secreted HMGB1 levels in AD (n=6 patients) vs. control (n=4) iNs from 3 experiments (mean and 27-75% Cl, two-tailed Welch's t-test, P−=0.053).

FIGS. 13A-13E show that CK2 levels and activity are higher in PD postmortem samples and AD patient-derived astrocytes; FIG. 13A shows that fluorescence signal increases over time as Csox peptide is phosphorylated in postmortem brain lysates (n=6 PD patients, n=6 controls, no lysate control in green (inset shows relevant time course to signal saturation); FIG. 13B shows that the fluorescence generated by phosphorylated Csox peptide is CK2-specific as it could be blocked by pretreatment of lysates with 20 μM of CX4945 for 30 min.; FIG. 13C is a Western blot showing no significant differences in CK2a1 or CK2a2 at the protein level in PD patients (P=0.15 for Ck2a1, P=0.41 for CK2a2, two-tailed t-test, n=6 each group); FIG. 13D shows that CSNK2A2 protein levels in AD are higher than controls, from the UPP Proteomics Study, box plot depicting mean log 2(abundance) (n=44-48, one-way ANOVA with Sidak's post-hoc test), CSNK2A1 and CSNK2B not significant; FIG. 13E shows HMGB1 protein levels in dorsolateral prefrontal cortex are higher in AD patients than controls, data from 2 Emory ADRC cohorts published at syn20821165, box plot depicting mean log 2(abundance) (n=9-10, two-tailed t-test, Brain1 P=0.037 and Brain2 P=0.026).

FIG. 14 is fluorescence images and a bar graph showing that CK2 Y255 phosphorylation is reduced by CHR and CX-4945 treatment in nuclei of primary human astrocytes.

FIG. 15 is fluorescence images and a bar graph showing that NF-κB transactivation domain (S529) phosphorylation is reduced by CHR and CX-4945 treatment in nuclei of primary human astrocytes.

FIG. 16 is a graph showing that rhamnetin inhibits phosphorylation of a commercial synthetic substrate peptide.

FIG. 17 is a bar graph showing CK2 activity quantified in n=2 control and n=3 BD patient-derived astrocyte cell line lysates in duplicate using the Cyclex CK2 ELISA assay (p=0.009, unpaired t-test).

FIG. 18 is a series of graphs showing that CK2A1 baseline protein levels (normalized to GAPDH) trend higher in BD astrocytes compared to controls (CT) (n=4 control, n=5 BD patients) and CK2A1 levels or phosphorylation of CK2 substrate CDC37-513 are not increased by IL1β activation in BD astrocytes.

FIG. 19 shows neuronal activity as measured by mean firing rate in iGluta neurons co-cultured with BD or CT astrocytes pre-treated with IL1β (D+ vs. D−), CHR and IL1β-pretreated (C+) BD astrocytes, vehicle (D+).

FIGS. 20A and 20B show that CSNK2A2 is upregulated in AD astrocytes at baseline; FIG. 20A shows that CK2 is a highly enriched kinase in differentially expressed genes in AD vs. CT astrocytes at baseline (n=3 CT, n=3 AD); FIG. 20B shows that CK2A2 is upregulated at the transcriptional level in AD patient-derived astrocytes (n=3 CT, n=3 AD).

FIG. 21 shows that AD iNs exhibit mitochondrial defects as reflected by reduced mitochondrial matrix protein ACO2 (unpaired t-test, p=0.03).

FIG. 22 shows that AD iNs exhibit increased CK2 activity and that CK2 inhibition with CHR increased LC3-II in the neurons.

FIG. 23 shows that AD patient iPSC-derived astrocytes exhibit hyperactivation in response to IL1β stimulation through increased IL-6 expression by qRT-PCR (n=5 AD, n=3 controls in duplicate).

FIG. 24 shows structural comparisons between adenosine triphosphate (ATP) and apigenin. Circled functional groups A, B, and C perform similar functions.

FIG. 25 is two crystal structures showing similarity in binding modes between adenosine diphosphate (ADP) and apigenin.

FIG. 26 shows molecular models of trimethoxyflavone and trimethoxyflavanone.

FIGS. 27-40 are exemplary synthesis schemes for certain disclosed compounds. In the schemes, solid reaction arrows indicate reactions that were performed, and dashed reaction arrows indicate prophetic reactions.

FIG. 41 shows results of an NF-κB-luciferase assay for anti-inflammatory activity of several known compounds, along with literature IC₅₀ values for CK2 inhibition.

FIGS. 42A and 42B show the generation of simple flavone analogues with improved physicochemical properties. CNS Score is a Schrodinger Maestro feature that predicts and ranks CNS drug-like properties from −2 to 2 (best). Shown in 42B are dose response curves for IL-6 secretion in astrocytes treated with IL1β and IN4.1 and IN4.2.

FIGS. 43A and 43B show dose-dependent reduction of IL-6 levels in primary astrocytes by several disclosed compounds, as well as solubility data.

FIG. 44 shows dose-dependent inhibition of CK2A1 activity by four of the disclosed compounds in vitro.

FIGS. 45A and 45B show antiviral activity of disclosed compounds against SARS-CoV2 in Vero-E6 cells (FIG. 45A) and cell viability (FIG. 45B).

FIGS. 46A and 46B show drug concentrations of a disclosed compound in mouse serum over time (FIG. 46A) and brain tissue (FIG. 46B).

FIGS. 47A-47C show drug concentrations of a disclosed compound in mouse serum over time (FIG. 47A) and liver tissue (FIG. 47B), as well as showing that administering the compound daily for 7 days did not affect body weight (FIG. 47C).

FIG. 48 shows blood-brain barrier permeability and anti-inflammatory activity of a disclosed compound as evidenced by reduction of IL6 and IL8 expression.

### DETAILED DESCRIPTION

This disclosure concerns embodiments of flavone derivatives, as well as methods of using the derivatives to inhibit CK2 enzyme activity. In some embodiments, the disclosed compounds exhibit an aqueous solubility ≥10 μM, blood-brain-barrier permeability (e.g., ≥100 ng/g in an organoid model), a high potency (IC₅₀≤100 nM) for CK2 in radiometric kinase assays, and/or an IC₅₀≤1 μM in a THP-1 monocyte NF-κB reporter assay or IL-6/IL-8 secretion in LPS-stimulated human peripheral blood mononuclear cells (PBMCs) or IL1β stimulated human astrocytes.

### I. Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). The presently disclosed compounds also include all isotopes of atoms present in the compounds, which can include, but are not limited to, deuterium, tritium, ¹⁸F, ¹⁴C, etc. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Acyl: An organic functional group having the general formula —C(O)R, where R is hydrogen, alkyl, heteroalkyl, haloalkyl, aliphatic, heteroaliphatic, aryl, or heteroaryl.

Administration: To provide or give a subject an agent, such as one or more compounds according to formulas I-IV provided herein, by any effective route. Exemplary routes of administration include, but are not limited to, oral, injection (such as subcutaneous, intramuscular, intradermal, intraperitoneal, intravenous, intraosseous, intracerebroventricular, intrathecal, and intratumoral), sublingual, rectal, transdermal, intranasal, vaginal and inhalation routes.

Aliphatic: A substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C≡C— double bond in an aliphatic chain, or up to one substituent for a carbon of a terminal methine group). A substituted aliphatic group includes at least one sp³-hybridized carbon or two sp²-hybridized carbons bonded with a double bond or at least two sp-hybridized carbons bonded with a triple bond. Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

Alkoxy: A radical (or substituent) having the structure —OR, where R is a substituted or unsubstituted alkyl. Methoxy (—OCH₃) is an exemplary alkoxy group.

Alkyl: A hydrocarbon group having a saturated carbon chain. The chain may be cyclic, branched or unbranched. Examples, without limitation, of alkyl groups include methyl, ethyl, propyl, butyl, pentyl, hexyl, heptyl, octyl, nonyl and decyl. The terms alkenyl and alkynyl refer to hydrocarbon groups having carbon chains containing one or more double or triple bonds, respectively.

Analog or Derivative: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a compound that is derived from a similar compound or a compound that can be imagined to arise from another compound, for example, if one atom is replaced with another atom or group of atoms. The latter definition is common in organic chemistry. In biochemistry, the word is used for compounds that at least theoretically can be formed from the precursor compound.

Aryl: A monovalent aromatic carbocyclic group of, unless specified otherwise, from 6 to 15 carbon atoms having a single ring (e.g., phenyl) or multiple condensed rings in which at least one ring is aromatic (e.g., quinoline, indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic or polycyclic (e.g., bicyclic, tricyclic or tetracyclic).

Azole: A 5-membered heterocyclic ring including a nitrogen atom and at least one other heteroatom (nitrogen, sulfur, or oxygen) as part of the ring. An oxazole is an azole including an oxygen atom, where the oxygen and the nitrogen are separated by one carbon.

Blood-brain barrier (BBB): A selective semipermeable border formed by endothelial cells lining the central nervous system microvasculature and allowing only certain agents circulating in the blood to pass through the barrier and contact central nervous system tissue (e.g., brain, spinal cord tissue).

Casein kinase 2 (CK2): (e.g., OMIM: 115440 and 115441) A serine/threonine-selective protein kinase responsible for phosphorylation of substrates in various pathways within a cell, and has been implicated in cell cycle control, DNA repair, regulation of the circadian rhythm, and other cellular processes. A member of enzyme class EC 2.7.11.1. CK2 typically appears as a tetramer of two a subunits and two β subunits. The terms CK2A, CK2a, and CK2α as used herein are interchangeable. Similarly, the terms CK2B, CK2b, and CK20 are interchangeable.

COVID-19: A contagious disease caused by severe acute respiratory syndrome coronavirus 2 (SARS-CoV-2). Symptoms of COVID-19 are variable, but often include fever, cough, fatigue, breathing difficulties, and loss of smell and taste. Symptoms can begin one to fourteen days after exposure to the virus. Around one in five infected individuals do not develop any symptoms. While most people have mild symptoms, some people develop acute respiratory distress syndrome (ARDS). ARDS can be precipitated by cytokine storms, multi-organ failure, septic shock, and blood clots. Longer-term damage to organs (in particular, the lungs and heart) has been observed. There is concern about a significant number of patients who have recovered from the acute phase of the disease but continue to experience a range of effects—known as long COVID—for months afterwards. These effects include severe fatigue, memory loss and other cognitive issues, low-grade fever, muscle weakness, and breathlessness. In some examples, one or more of the according to formulas I-IV provided herein are used to treat COVID-19.

Excipient: A physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Heteroaliphatic: An aliphatic compound or group having at least one carbon atom in the chain and at least one heteroatom, i.e., one or more carbon atoms has been replaced with an atom having at least one lone pair of electrons, typically nitrogen, oxygen, phosphorus, silicon, or sulfur. Heteroaliphatic compounds or groups may be substituted or unsubstituted, branched or unbranched, cyclic or acyclic, and include “heterocycle”, “heterocyclyl”, “heterocycloaliphatic”, or “heterocyclic” groups. Heteroalkyl refers to an alkyl or cycloalkyl radical having at least one carbon atom in the chain and containing at least one heteroatom, such as N, O, S, or S(O)ₙ (where n is 1 or 2).

Heteroaryl: An aromatic compound or group having at least one heteroatom, i.e., one or more carbon atoms in the ring has been replaced with an atom having at least one lone pair of electrons, typically nitrogen, oxygen, phosphorus, silicon, or sulfur.

Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.

SARS-CoV-2: Also known as Wuhan coronavirus or 2019 novel coronavirus, SARS-CoV-2 is a positive-sense, single stranded RNA virus of the genus betacoronavirus that has emerged as a highly fatal cause of severe acute respiratory infection. Includes the original SARS-Cov-2 virus or variants thereof (such as the UK variant B1.1.17, the South Africa variant B.1.351, and the Brazil variant P.1). The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms, background and genetic tests such as reverse transcription-polymerase chain reaction (rRT-PCR), and antibody tests. The test can be done on patient samples such as respiratory or blood samples.

In some examples, one or more of the according to formulas I-IV provided herein are used to treat one or more symptoms of a SARS-CoV-2 infection.

Stereoisomers: Isomers that have the same molecular formula and sequence of bonded atoms, but which differ only in the three-dimensional orientation of the atoms in space.

Subject: An animal (human or non-human) subjected to a treatment, observation or experiment. Includes both human and veterinary subjects, including human and non-human mammals, such as rats, mice, cats, dogs, pigs, horses, cows, and non-human primates. In some examples, the treated subject has an inflammatory disease or a disease that causes undesired inflammation, such as cancer, cardiac hypertrophy, cystic fibrosis, a neurodegenerative disease, bipolar disorder, depression, a viral infection (such as SARS-CoV-2), obesity, diabetes mellitus, atherosclerosis, epilepsy, or any combination thereof.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto.

Tautomers: Constitutional isomers of organic compounds that differ only in the position of the protons and electrons, and are interconvertible by migration of a hydrogen atom. Tautomers ordinarily exist together in equilibrium.

Therapeutically effective amount or dose: An amount sufficient to provide a beneficial, or therapeutic, effect to a subject or a given percentage of subjects.

Triazine: A 6-membered nitrogen-containing heterocycle including three nitrogen atoms.

Treating or treatment: With respect to disease, either term includes (1) preventing the disease, e.g., causing the clinical symptoms of the disease not to develop in an animal that may be exposed to or predisposed to the disease but does not yet experience or display symptoms of the disease, (2) inhibiting the disease, e.g., arresting the development of the disease or its clinical symptoms, or (3) relieving the disease, e.g., causing regression of the disease or its clinical symptoms.

### II. Flavone Derivatives

Embodiments of flavone derivatives are disclosed. The flavone derivative may be a compound according to formula I, or a pharmaceutically acceptable salt, hydrate, stereoisomer, or tautomer thereof:

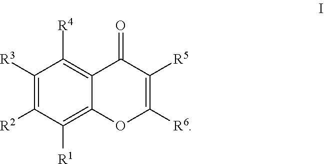

With respect to formula I, R¹ is H, halo, —OR^{a}, substituted or unsubstituted aryl, substituted or unsubstituted heteroaryl, —SF₅, or -L-Q where L is a linker and Q is an E3-ligase binding moiety; R² is H, —O(CH₂)ₘR^{a} where m is an integer greater than or equal to zero, or -L-Q. R³ is H, halo, —OR^{a},

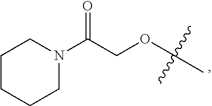 [SMILES: *OCC(=O)N1CCCCC1]

a hydrophobic group (e.g. aliphatic, substituted aliphatic (e.g., —CF₃), —SF₅), or -L-Q. R⁴ and R⁵ independently are H or —OR^{a}. R^{a} is H, substituted or unsubstituted aliphatic, substituted or unsubstituted heteroaliphatic, or acyl. R⁶ is —(CH₂)ₙ—R^{b}, —C≡C—R^{b}, substituted aliphatic, H, or halo, where n is an integer from 1-10 and R^{b} is substituted or unsubstituted heteroaliphatic or —OR^{a}, or R^{b} is R¹¹, where R¹¹ is substituted or unsubstituted heteroaryl or substituted or unsubstituted aryl; or R⁶ is

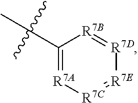

where R^{7A} N or CH, R^{7B} is N or CH, R^{7C} is N or C—R⁸, R^{7D} is N or C—R¹⁰, and R^{7E} is C—R⁹ or N⁺—O⁻, and 0, 1, or 2 of R^{7A}—R^{7D} is N; R⁸ is H, —OR^{a}, substituted or unsubstituted alkyl, or halo, R⁹ is —OR^{a}, —CN, —C(O)OR^{a}, or azole, and R¹⁰ is H, alkynyl, -alkynyl-R¹¹, -alkynyl-heteroaliphatic, halo, —OR^{a}, or a hydrophobic group, or R⁸/R⁹ or R⁹/R¹⁰ together with the atoms to which they are bound form a substituted or unsubstituted heteroaliphatic or heteroaryl ring. In some embodiments, if R^{7A} and R^{7B} are both CH, (i) R³ is not alkyl, or (ii) R⁹ and R¹⁰ together with the atoms to which they are bound form a substituted or unsubstituted heteroaliphatic or heteroaryl ring including at least one N atom in the ring, or (iii) R¹ is furan substituted with —C(O)OR^{a}, or (iv) one of R^{7C} or R^{7D} is N, or (v) R^{7E} is N⁺—O⁻.

In any of the foregoing or following embodiments, R^{a} is H, substituted or unsubstituted aliphatic, substituted or unsubstituted heteroaliphatic, or acyl. In some embodiments, R^{a} is H, substituted or unsubstituted C₁-C₁₀ alkyl, substituted or unsubstituted C₁-C₁₀ heteroalkyl including one or more heteroatoms, or acyl. In certain embodiments, R^{a} is H, unsubstituted C₁-C₁₀ alkyl, C₁-C₁₀ haloalkyl, or acetyl. In some examples, R^{a} is H, methyl, difluoromethyl, trifluoromethyl, or fluoromethyl. In certain implementations, R^{a} is H or methyl. In an independent embodiment, R^{a} is cycloheteroalkyl, such as 1,4-oxazinyl.

In any of the foregoing or following embodiments, R¹ is H, halo, —OR^{a}, substituted or unsubstituted aryl, substituted or unsubstituted heteroaryl, —SF₅, or -L-Q, where R^{a}, L, and Q are as previously defined. In some embodiments, R¹ is H, halo, C₁-C₅ alkoxy, substituted aryl, substituted heteroaryl, —SF₅, or -L-Q. In certain embodiments, R¹ is H, Br, Cl, substituted aryl or substituted heteroaryl. The substituted aryl may be a substituted phenyl, e.g., phenyl substituted with halo, alkynyl, or -alkynyl-R¹¹. In some embodiments, the phenyl is substituted at the meta position relative to its attachment to the 1-benzopyran-4-one moiety. In certain examples, the phenyl is substituted at the meta position with halo (e.g., chloro), ethynyl, or

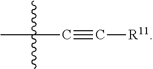

In some embodiments, R¹ is substituted heteroaryl, e.g., a substituted pyridine or substituted furan, such as halo-substituted pyridine or furan substituted with —C(O)OR^{a}. In some implementations, R¹ is

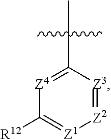

where each of Z¹-Z⁴ is CH or one of Z¹—Z⁴ is N and the others of Z¹-Z⁴ are CH; and R¹² is halo, alkynyl, or -alkynyl-R¹¹ where R¹¹ is as previously described. In certain implementations, R¹² is halo, alkynyl, or H, such as Cl, ethynyl, or H. In some examples, R¹ is furan substituted with —C(O)OCH₃. Exemplary R¹ groups include: Cl, Br, H,

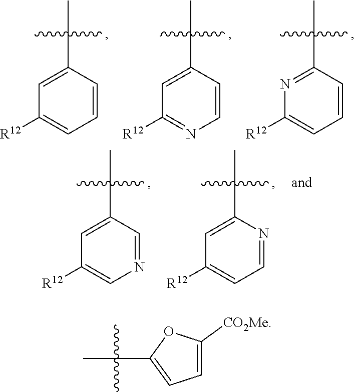

In any of the foregoing or following embodiments, R² is H, —O(CH₂)ₘR^{a} where m is an integer greater than or equal to zero, or -L-Q, where R^{a}, L, and Q are as previously defined. In some embodiments, m is an integer from 0 to 10. In certain implementations, m is 0, 1, 2, 3, or 4. In certain embodiments, R² is H or —O(CH₂)ₘR^{a}, where m is 0 and R^{a} is methyl or acyl, or m is 1, 2, or 3, and R^{a} is cycloheteroalkyl, sums 1,4-oxazinyl. In some examples, m is 2, and R^{a} is 1,4-oxazinyl, i.e., R² is

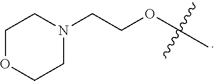 [SMILES: *OCCN1CCOCC1]

In certain implementations, R² is H, methoxy, or acetoxy.

In any of the foregoing or following embodiments, R³ is H, halo, —OR^{a},

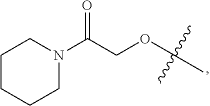 [SMILES: *OCC(=O)N1CCCCC1]

a hydrophobic group, or -L-Q, where R^{a}, L, and Q are as previously defined. Exemplary hydrophobic groups include, but are not limited to, aliphatic, haloaliphatic, and pentafluorosulfanyl groups. In some examples, the hydrophobic group is alkyl, haloalkyl (e.g., trifluoromethyl), or pentafluorosulfanyl. In some embodiments, R³ is H, Br, Cl, F,

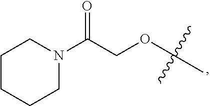 [SMILES: *OCC(=O)N1CCCCC1]

trifluoromethyl, or pentafluorosulfanyl.

In any of the foregoing or following embodiments, R¹, R², or R³ may be -L-Q where L is a linker and Q is an E3-ligase binding moiety. In such embodiments, the compound is a targeted degrader, or proteolysis-targeting chimera (PROTAC), capable of degrading CK2 enzyme. Generally, where the compound includes -L-Q, only one of R¹, R², and R³ is -L-Q, and the others are as previously defined. L may be any linker. Suitable linkers include, but are not limited to, heteroaliphatic linkers (e.g., PEG-based linkers) or aliphatic linkers. Q is an E3-ligase binding moiety. Exemplary E3-ligase binding moieties includes, but are not limited to the VHL ligase-binding moiety, the cereblon ligase-binding moiety, the IAP ligase-binding moiety, the MDM2 ligase-binding moiety, and derivatives thereof. In some embodiments, Q is:

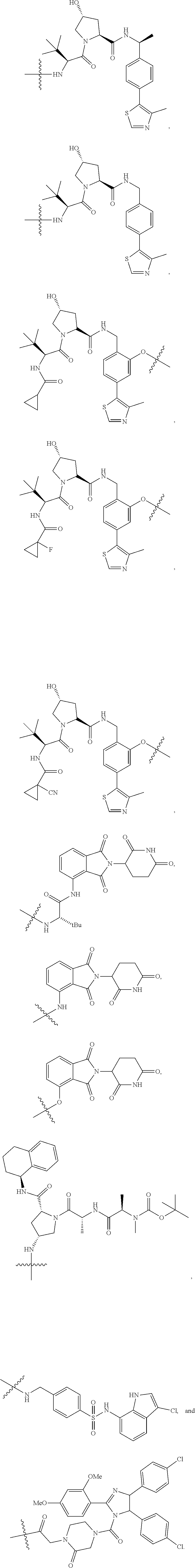

In certain embodiments, -L-Q is:

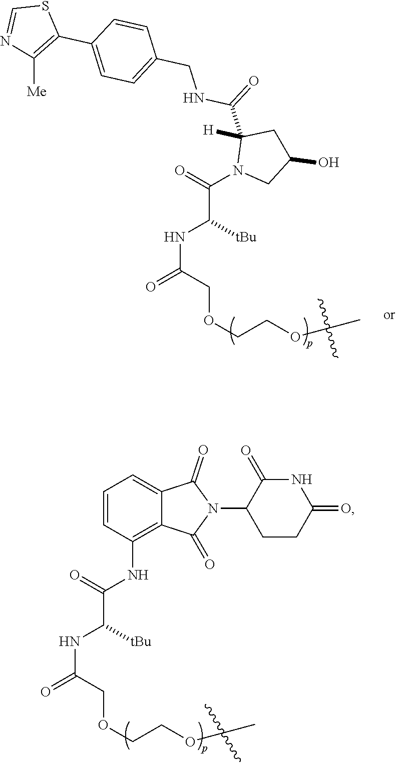

where p is an integer from 2 to 7.

In any of the foregoing or following embodiments, R⁴ and R⁵ independently are H or —OR^{a} where R^{a} is as previously defined. In some embodiments, R⁴ is H, C₁-C₅ alkoxy, or acetoxy. In certain embodiments, R⁴ is H, methoxy, or acetoxy. In some embodiments, R⁵ is H, hydroxy, or methoxy. In some implementations, R⁴ and R⁵ are both H.

In any of the foregoing or following embodiments, when R² is other than H, then R¹ and R³ may be H. In any of the foregoing or following embodiments, when R³ is other than H, then R² and R⁴ may be H. In one embodiment, R¹ and R³ are H, and R² and R⁴ are other than H. In some examples, R² and R⁴ are OH, methoxy or acetoxy, and R¹ and R³ are H. In an independent embodiment, R¹ and R³ are other than H, and R² and R⁴ are H. In some examples, R¹ and R³ are halo (e.g., bromo or chloro), and R² and R⁴ are H, OH, or methoxy. In another independent embodiment, one of R¹-R⁴ is other than H, and the others of R¹-R⁴ are H. In some examples, R² is methoxy or —O(CH₂)₂R^{a}, where R^{a} is 1,4-oxazinyl, and R¹, R³, and R⁴, are H. In other examples, R³ is,

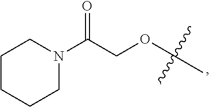 [SMILES: *OCC(=O)N1CCCCC1]

and R¹, R², and R⁴, are H. In still other examples, R¹ is

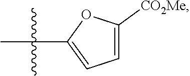 [SMILES: *c1ccc(C(=O)OC)o1]

and R²-R⁴ are H. In some implementations, R³ is H, halo or —SF₅, R¹ is H or

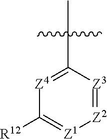

where R¹² is halo, and R² and R⁴ are H. In certain implementations, R² is -L-Q, R¹ and R³ are halo, H, or methoxy, and R⁴ is H. In yet another independent embodiment, R¹-R⁴ are H.

In any of the foregoing or following embodiments, R⁶ is —(CH₂)ₙ—R^{b}, —C≡C—R^{b}, substituted aliphatic, H, or halo, where n is an integer from 1-10 and R^{b} is substituted or unsubstituted heteroaliphatic or —OR^{a}, or R^{b} is R¹¹, where R¹¹ is substituted or unsubstituted heteroaryl or substituted or unsubstituted aryl, or R⁶ is

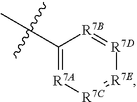

where R^{7A} N or CH, R^{7B} is N or CH, R^{7C} is N or C—R⁸, R^{7D} is N or C—R¹⁰, and R^{7E} is C—R⁹ or N⁺—O⁻, and 0, 1, or 2 of R^{7A}—R^{7D} is N. R⁸ is H, —OR^{a}, substituted or unsubstituted alkyl, or halo; R⁹ is —OR^{a}, —CN, —C(O)OR^{a}, or azole; and R¹⁰ is H, alkynyl, -alkynyl-R¹¹, -alkynyl-heteroaliphatic, halo, —OR^{a}, or a hydrophobic group; or R⁸/R⁹ or R⁹/R¹⁰ together with the atoms to which they are bound form a substituted or unsubstituted heteroaliphatic or heteroaryl ring. R^{a} and R¹¹ are as previously defined. In some embodiments, if R^{7A} and R^{7B} are both CH, then (i) R³ is not alkyl, or (ii) R⁹ and R¹⁰ together with the atoms to which they are bound form a substituted or unsubstituted heteroaliphatic or heteroaryl ring including at least one N atom in the ring, or (iii) R¹ is furan substituted with —C(O)OR^{a}, or (iv) one of R^{7C} or R^{7D} is N, or (v) R^{7E} is N⁺—O⁻.

In some embodiments, R⁶ is —(CH₂)ₙ—R^{b} or —C≡C—R^{b} where n is an integer from 1-10 and R^{b} is substituted or unsubstituted heteroaliphatic or —OR^{a}, or R^{b} is R¹¹, where R¹¹ is substituted or unsubstituted heteroaryl or substituted or unsubstituted aryl. In certain embodiments, n is 1, 2, or 3. In some examples, n is 2. In some embodiments, R^{b} is an azole (e.g., an oxazole, oxadiazole, furazan, imidazole), triazine, tetrahydrofuran, furan, unsubstituted or substituted phenyl (e.g., hydroxyphenyl), pyridine, or pyrimidine. When R^{b} is an azole, the attachment point may be through a ring nitrogen atom or a ring carbon atom.

In some embodiments, R⁶ is substituted aliphatic, halo (e.g., Cl, Br, or F), or H. In certain implementations, R⁶ is hydroxy or alkoxy, such as C₁-C₅ hydroxyalkyl, e.g., hydroxyethyl (—CH₂CH₂OH). In an independent embodiment, R⁶ is —(CH₂)₂—R^{b} where R^{b} is an azole, triazine, or tetrahydrofuran.

In some embodiments, R⁶ is

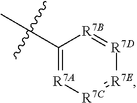

where R^{7A} N or CH, R^{7B} is N or CH, R^{7C} is N or C—R⁸, R^{7D} is N or C—R¹⁰, and R^{7E} is C—R⁹ or N⁺—O⁻, and 0, 1, or 2 of R^{7A}—R^{7D} is N. R⁸ is —OR^{a}, H, substituted or unsubstituted alkyl, or halo; R⁹ is —OR^{a}, —CN, —C(O)OR^{a}, or azole; and R¹⁰ is H, alkynyl, -alkynyl-R¹¹, -alkynyl-heteroaliphatic, halo, —OR^{a}, or a hydrophobic group; or R⁸/R⁹ or R⁹/R¹⁰ together with the atoms to which they are bound form a substituted or unsubstituted heteroaliphatic or heteroaryl ring. In some embodiments, R⁶ is substituted phenyl or pyridinyl. In some embodiments, R⁶ is substituted phenyl or pyridinyl, where R⁹ is cyano, hydroxyl, —COOH, methoxy, acetoxy, azole (e.g., tetrazole, triazole, or diazole), or difluoromethoxy. In one embodiment, R^{7A} is N or CH, R^{7B} is N or CH, R^{7C} is N or C—R⁸ where R⁸ is H, —OCH₃, —CH₃, or halo, R^{7D} is N or C—R¹⁰ where R¹⁰ is H, halo, —CH₃, or

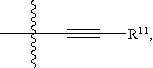

R^{7E} is C—R⁹ or N⁺—O⁻ where R⁹ is —OH, —OCH₃, —CO₂H, or —CN, and 0, 1, or 2 of R^{7A}—R^{7D} is N. In an independent embodiment, R^{7A} N or CH, R^{7B} is N or CH, R^{7C} is N or CH, R^{7D} is N or CH, R^{7E} is C—R⁹ where R⁹ is azole, and 0, 1, or 2 of R^{7A}—R^{7D} is N. In another independent embodiment, R⁶ is substituted or unsubstituted para-pyridyl N-oxide. In some implementations, R⁸ and R¹⁰ are H. In other implementations, one of R⁸ and R¹⁰ is H and the other of R⁸ and R¹⁰ is methoxy, halo, alkynyl, -alkynyl-R¹¹, -alkynyl-heteroaliphatic, or substituted or unsubstituted alkyl. In still other implementations, both R⁸ and R¹⁰ are other than H. For instance, R⁸ and R¹⁰ independently may be halo, or substituted or unsubstituted alkyl, such as methyl. In some embodiments, R^{7A} is N, R^{7B} is CH, R⁸ is H or methoxy, R⁹ is hydroxy, methoxy, or acetoxy, and R¹⁰ is H, ethynyl, or methyl. In some embodiments, R^{7A} and R^{7B} are CH, R¹⁰ is H, R⁹ is hydroxy, methoxy, or —COOH, and R¹⁰ is alkynyl or -alkynyl-R¹¹ where R¹¹ is as previously defined. In some embodiments, R^{7A} is CH, R^{7B} is N, R⁸ is H or methoxy, and R¹⁰ is H, ethynyl, or halo.

In certain embodiments, (i) R¹⁰ is H and R⁸/R⁹ together with the atoms to which they are bound form a substituted or unsubstituted heteroaliphatic or heteroaryl ring, or (ii) R⁸ is H and R⁹/R¹⁰ together with the atoms to which they are bound form a substituted or unsubstituted heteroaliphatic or heteroaryl ring. Exemplary polycyclic R⁶ groups include, but are not limited to

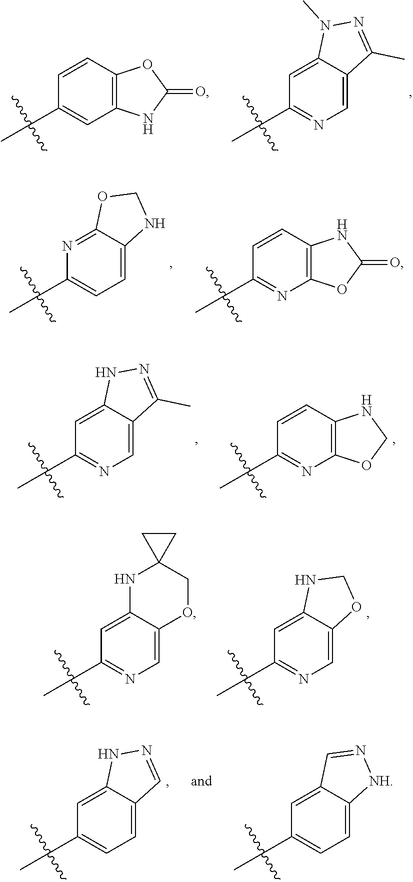

In some embodiments, the flavone derivative is a compound according to one of formulas II-IV:

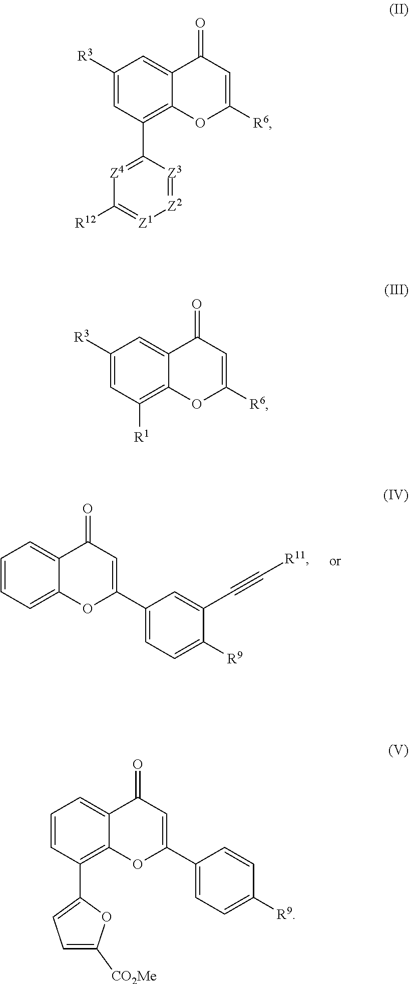

In certain embodiments, the flavone derivative is a compound according to one of formulas IIA-IIE or IIIA-IIIF:

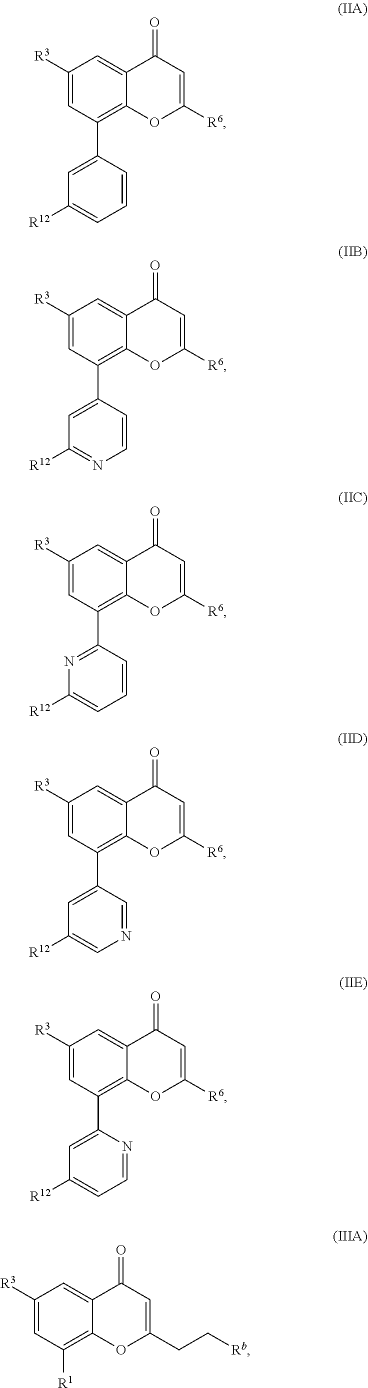

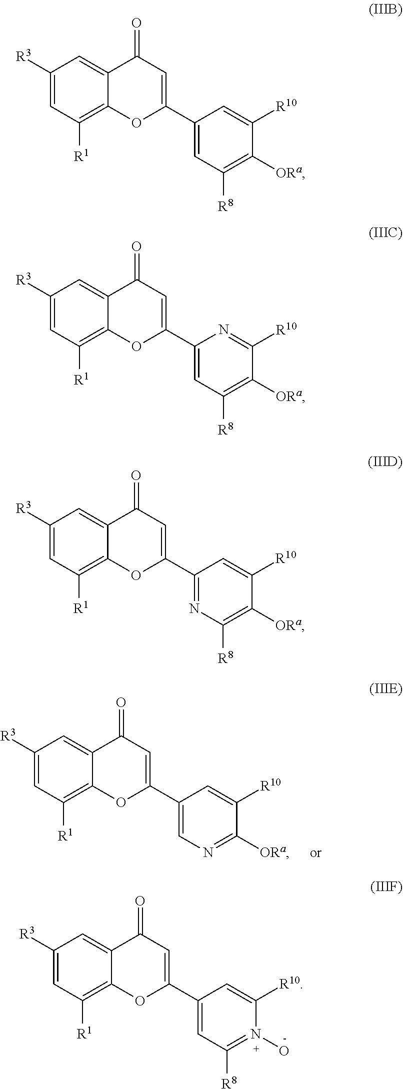

R¹, R³, R⁶, R⁸-R¹¹, R^{a} and R^{b} are as previously defined, and Z¹-Z⁴ are CH or one of Z¹-Z⁴ is N and the others of Z¹-Z⁴ are CH. R¹² is halo, H, alkynyl, or -alkynyl-R¹¹. In certain embodiments, R³ is halo or H; R⁹ is —CN, or —OH; R⁸ is H, substituted or unsubstituted alkyl (e.g. CH₃, CF₃), halo, or —OR^{a}; R¹⁰ is ethynyl, substituted or unsubstituted alkyl (e.g., C₁-C₄ alkyl), halo, or —OR^{a}; R¹¹ is heteroaryl; R¹² is halo, H, or ethynyl; R^{a} is H, alkyl (e.g., C₁-C₄ alkyl), or acyl; and R^{b} is —OH or R¹¹.

In some embodiments, the compound has a structure according to any one of formulas IIA-IIE or IIIA-IIIB, where R³ is halo or H; R⁶ is H, —(CH₂)ₙ—R^{b}, or

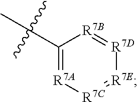

R⁸ is H, substituted or unsubstituted alkyl (e.g. CF₃), halo, or —OR^{a}; R⁹ is —CN, or —OH; R¹⁰ is ethynyl, substituted or unsubstituted alkyl, halo, or —OR^{a}; R¹² is halo, H, or ethynyl; R^{a} is H, alkyl, or acyl; and R^{b} is —OH or R¹¹, where R¹¹ is heteroaryl. In some implementations, the compound has a structure according to formula IIA where R³ is Cl or H, R¹² is Cl, and R⁶ is H,

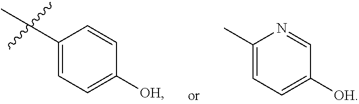

In some implementations, the compound has a structure according to formula IIB or IIIC where R¹ and R³ are Br, R¹⁰ is —CF₃ or —OCH₃, and R^{a} is H.

In any of the foregoing or following embodiments, R¹¹ may be:

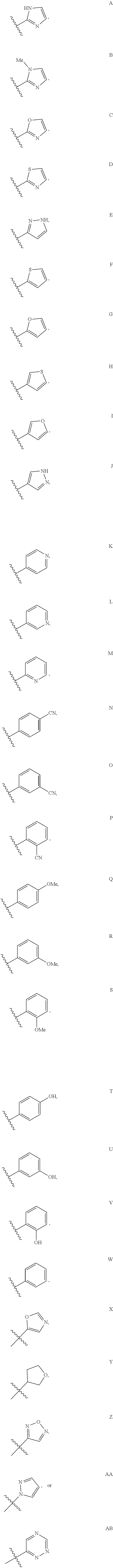

Some embodiments of the compounds have an aqueous solubility ≥10 μM, such as ≥5 μM, ≥10 μM, ≥50 μM, ≥100 μM, or ≥200 μM. Presence of polar substituents may enhance aqueous solubility and/or bioavailability. Certain embodiments of the compounds are capable of passing through the blood-brain barrier. In some embodiments, the compound has an aqueous solubility ≥10 μM and is capable of passing through the blood-brain barrier. In some embodiments, the compound includes one or more alkoxy groups at R¹⁻⁵, R⁸, or R⁹, where the alkoxy groups may provide enhanced metabolic stability in vivo compared to compounds that include no alkoxy groups. In some embodiments, the alkoxy groups are methoxy groups.

In some embodiments, pyridyl derivatives (with N at C2′ (R^{7A}) or C6′ (R^{7B})) and/or derivatives including hydroxy groups (e.g., at C3) or other substituent heteroatoms may exhibit increased aqueous solubility. The substituent at the C4′ (R⁹) position may interact with Lys68 in the CK2 binding pocket. In certain embodiments, including no more than one H-bond donor may facilitate the ability to cross the blood-brain barrier.

Exemplary compounds according to formulas I-IV include, but are not limited to, the compounds in Table 1, as well as pharmaceutically acceptable salts, hydrates, stereoisomers, or tautomers thereof.

TABLE 1

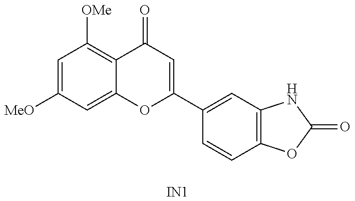

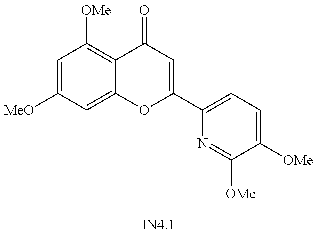

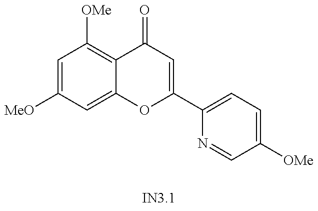

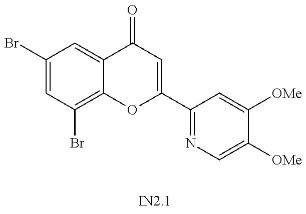

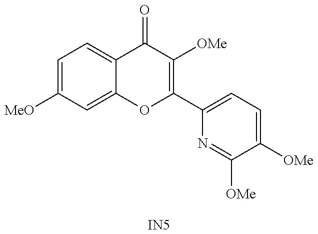

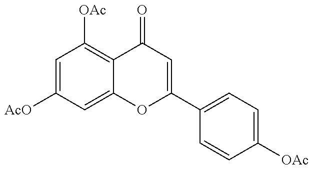

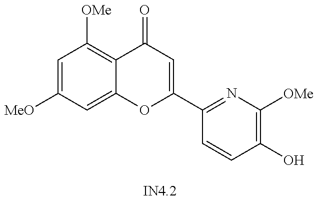

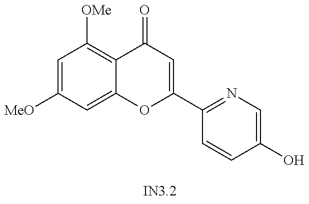

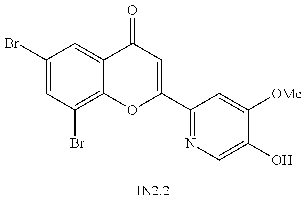

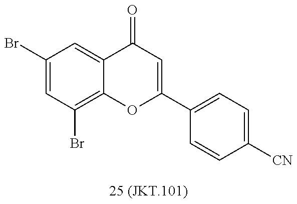

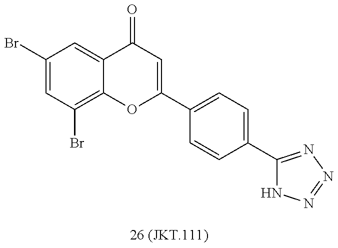

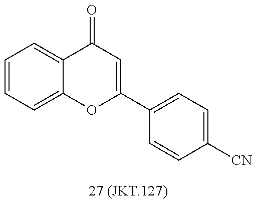

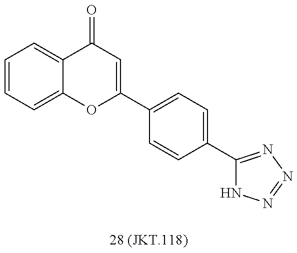

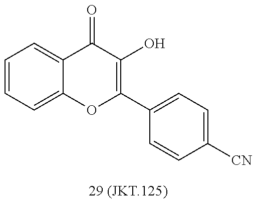

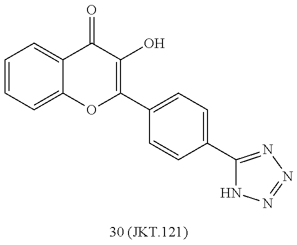

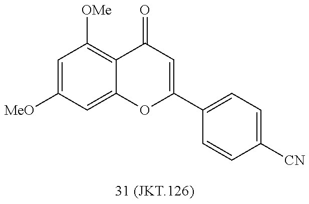

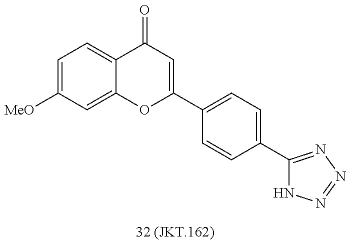

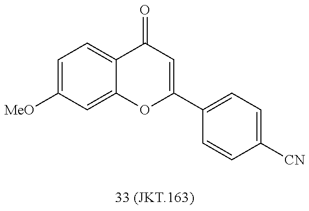

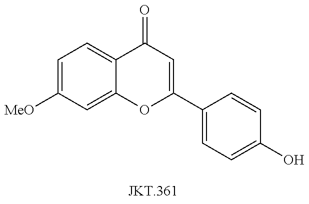

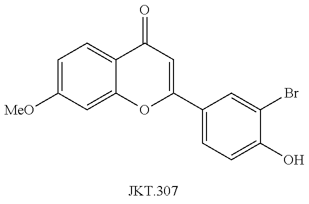

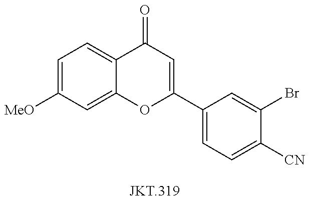

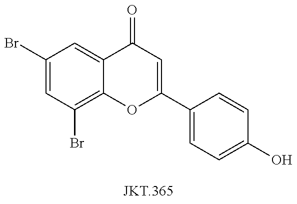

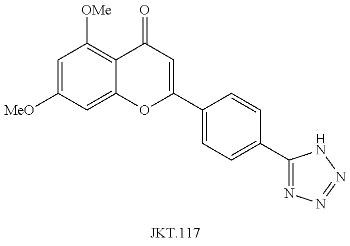

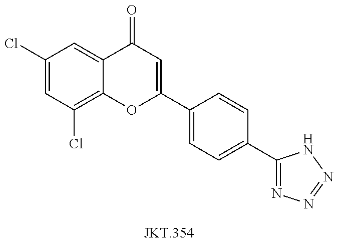

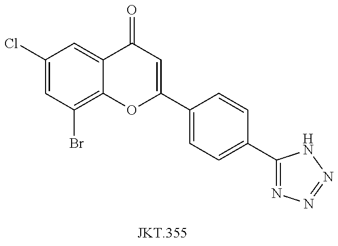

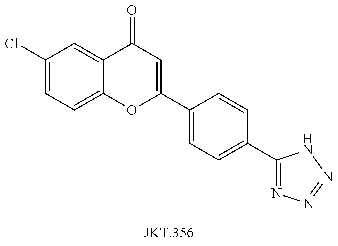

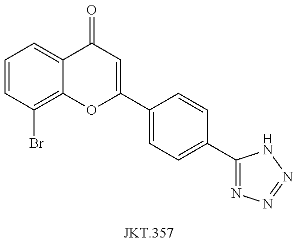

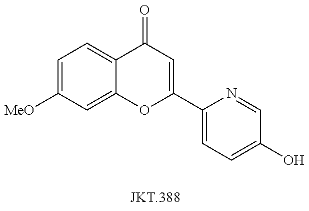

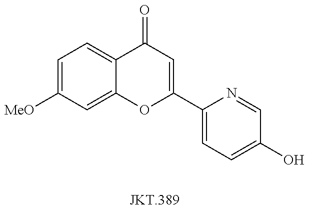

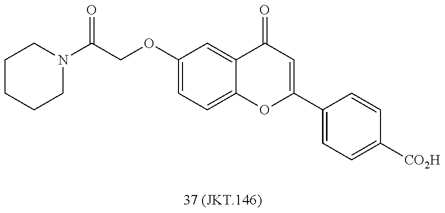

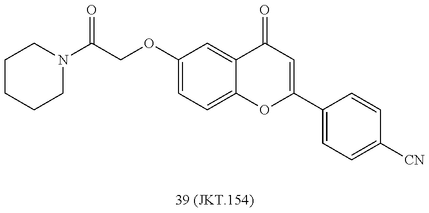

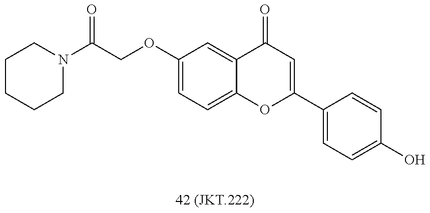

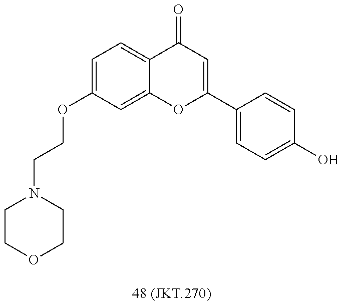

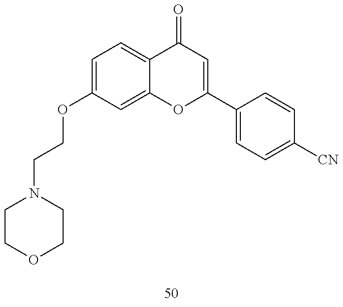

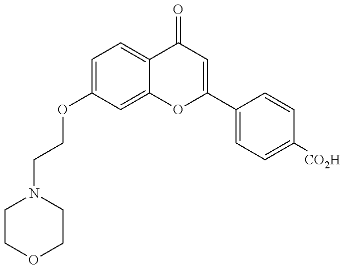

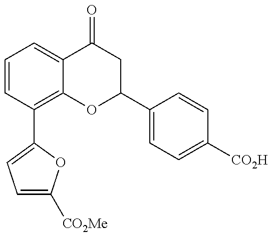

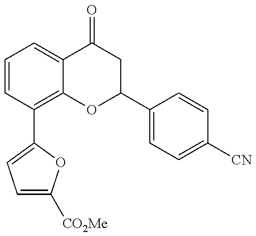

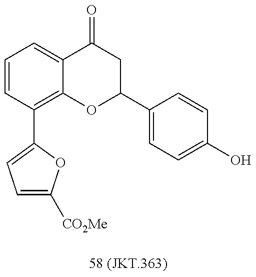

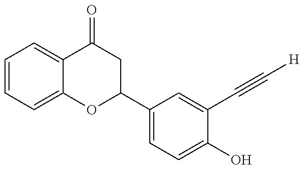

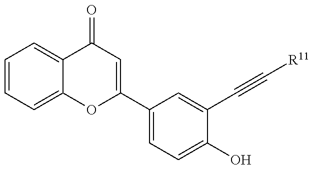

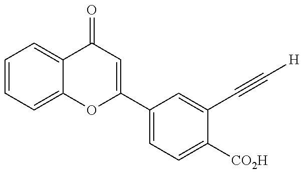

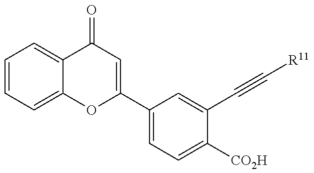

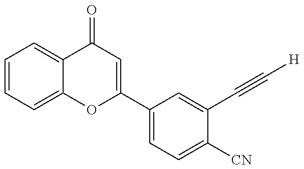

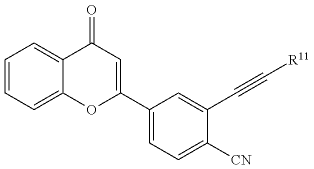

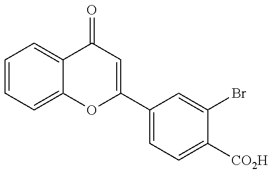

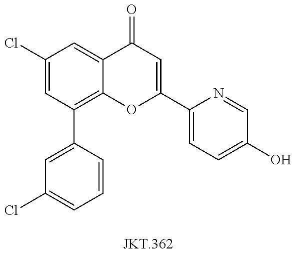

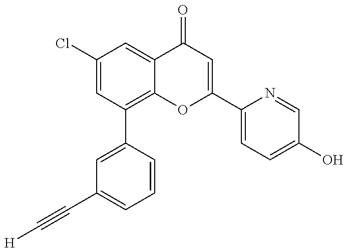

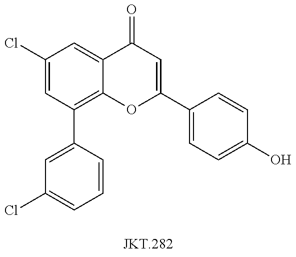

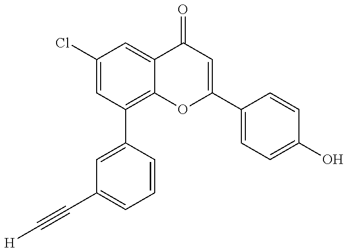

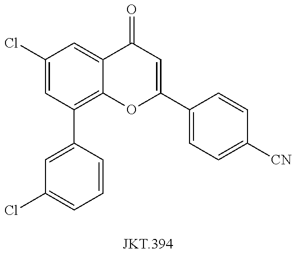

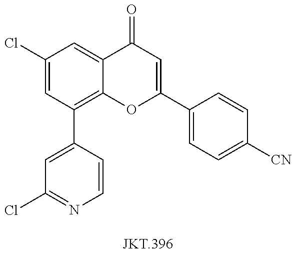

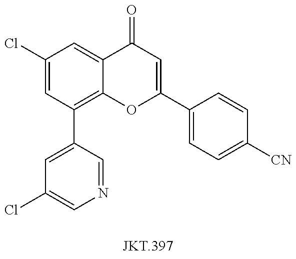

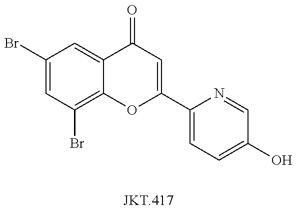

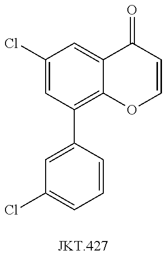

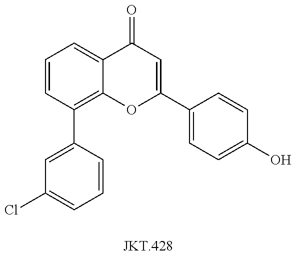

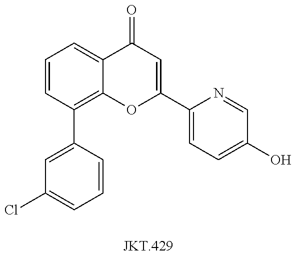

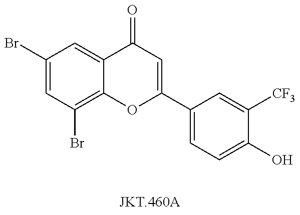

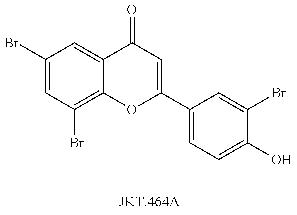

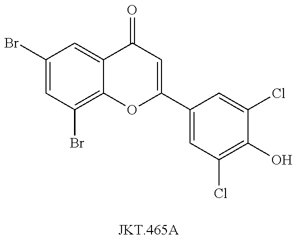

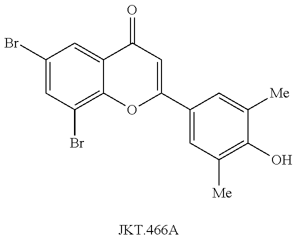

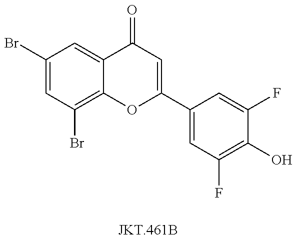

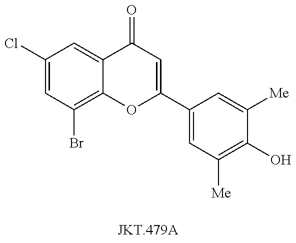

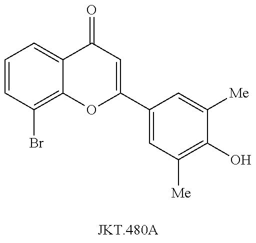

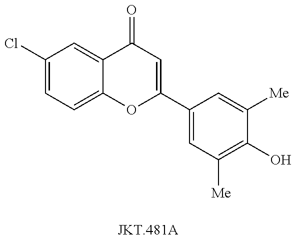

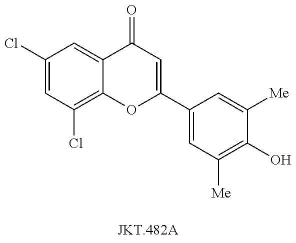

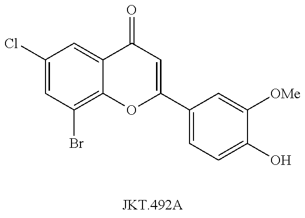

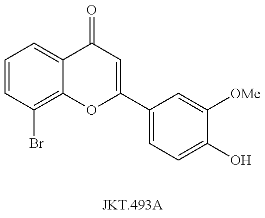

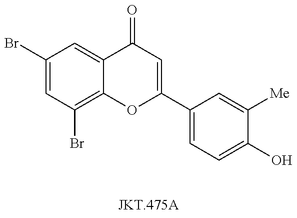

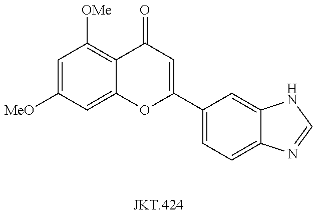

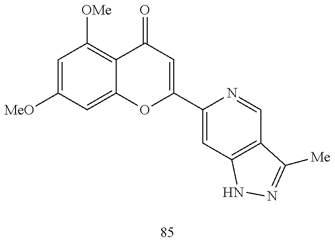

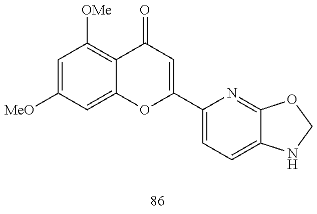

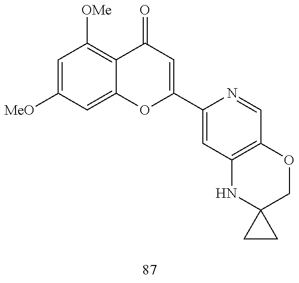

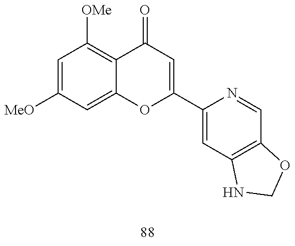

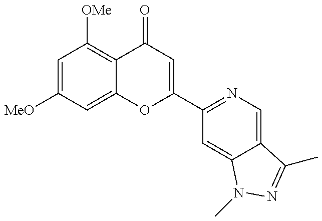

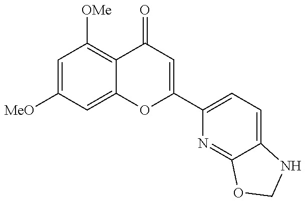

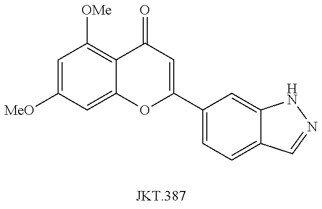

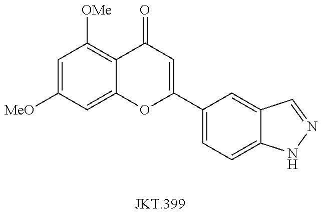

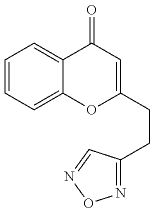

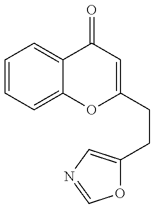

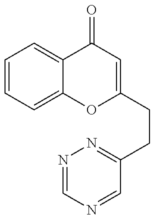

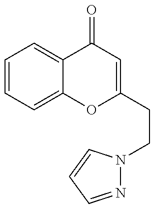

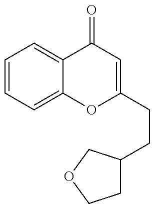

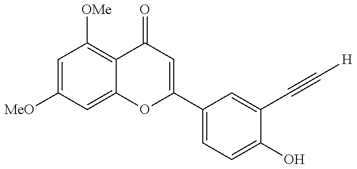

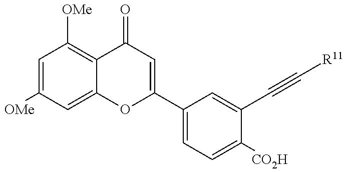

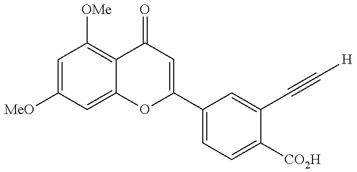

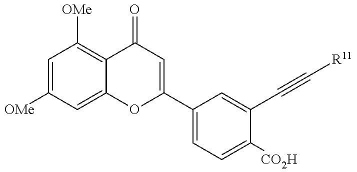

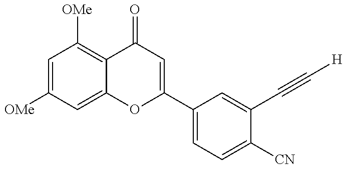

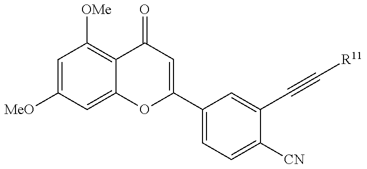

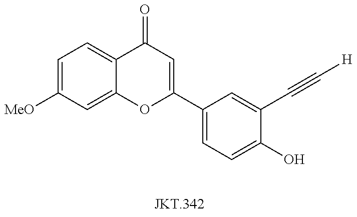

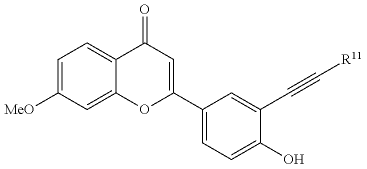

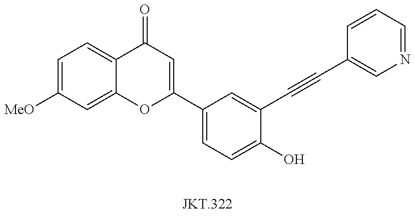

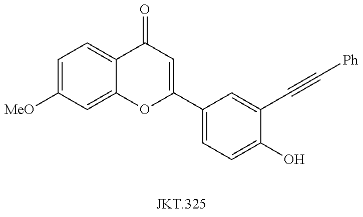

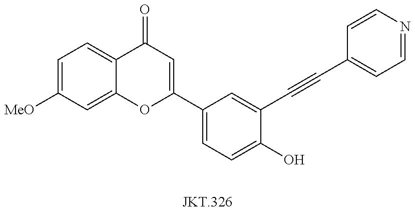

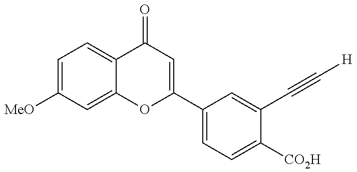

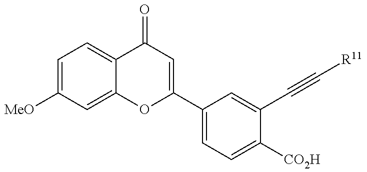

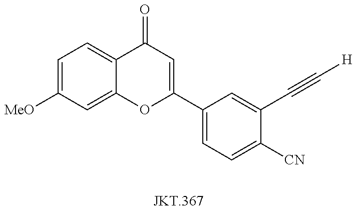

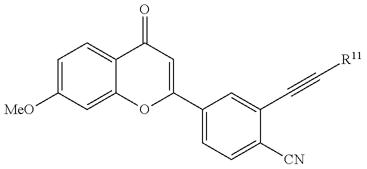

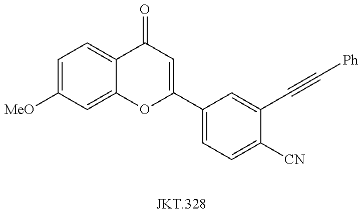

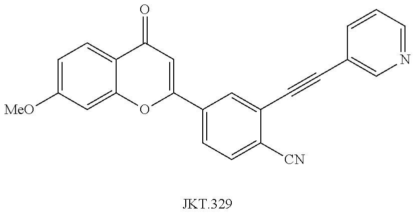

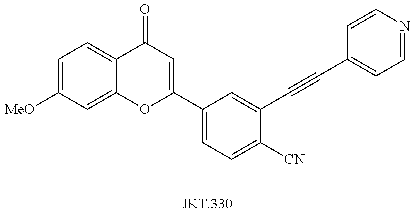

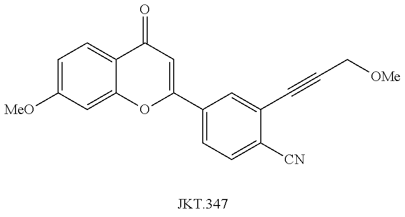

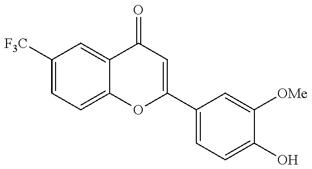

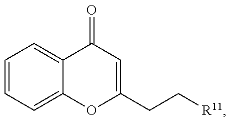

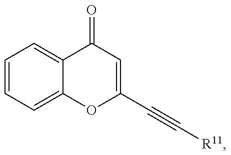

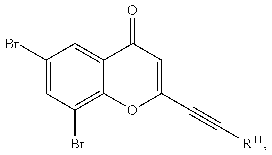

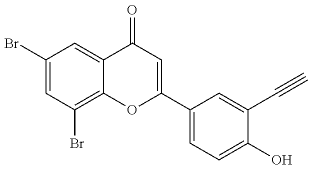

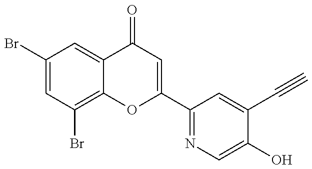

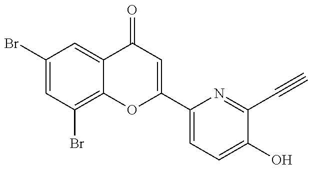

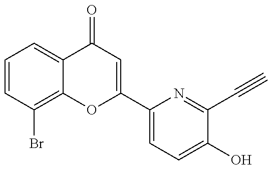

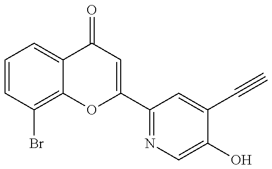

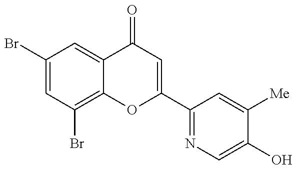

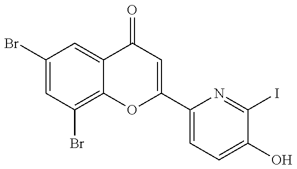

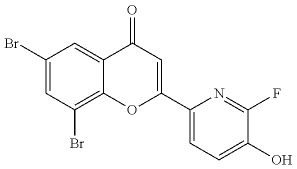

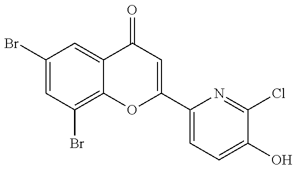

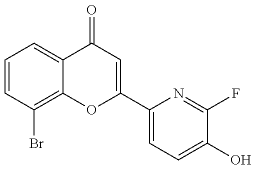

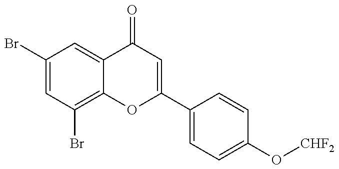

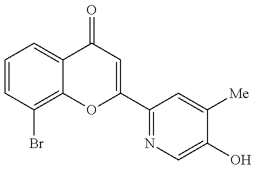

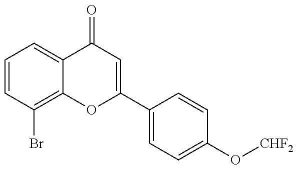

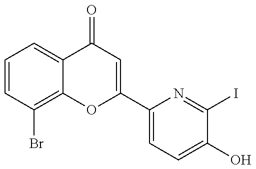

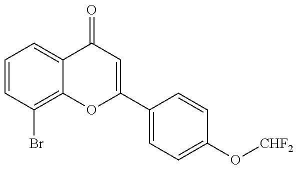

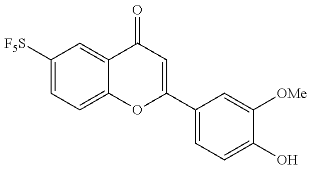

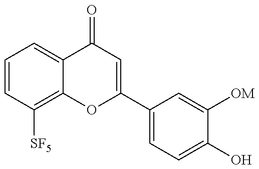

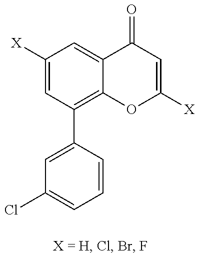

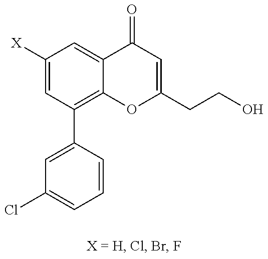

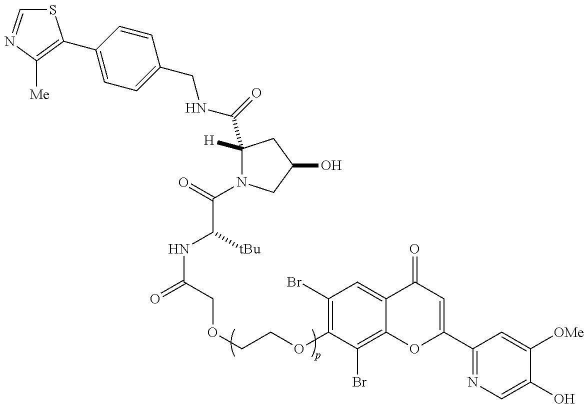

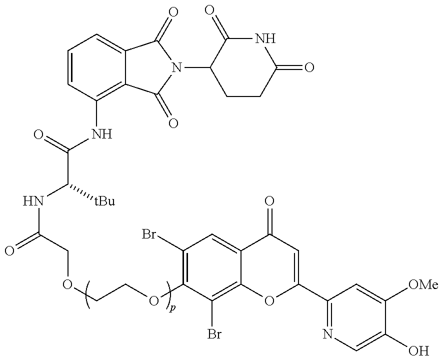

where R¹¹ is substituted or unsubstituted heteroaryl or substituted or unsubstituted aryl, and p is 2, 3, 4, 5, 6, or 7. Exemplary compounds including R¹¹ in Table 1 include, but are not limited to:

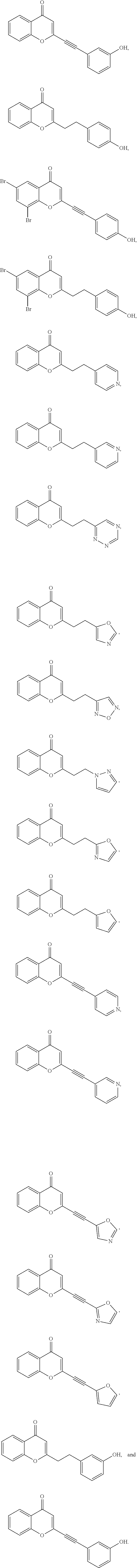

### III. Pharmaceutical Compositions

The disclosure also encompasses pharmaceutical compositions comprising one or more of the disclosed flavone derivatives. A pharmaceutical composition comprises a compound as disclosed herein and a pharmaceutically acceptable excipient.

The compounds described herein can be used to prepare therapeutic pharmaceutical compositions. The compounds may be added to the compositions in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and b-glycerophosphate. Suitable inorganic salts may also be formed, including hydrochloride, halide, sulfate, nitrate, bicarbonate, and carbonate salts.

Pharmaceutically acceptable salts may be obtained using procedures known to persons of ordinary skill in the art, for example by reacting a sufficiently basic compound, such as an amine, with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human or veterinary patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 2% to about 60% of the weight of a given unit dosage form. The amount of active compound in such therapeutically useful compositions is such that an effective dosage level can be obtained.

The tablets, troches, pills, capsules, and the like may also contain one or more of the following excipients: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

### IV. Methods of Use

Casein kinase 2 (CK2) is a potent driver of inflammation, such as neuroinflammation. CK2 is a potent driver of glial inflammation, especially in Alzheimer's disease (AD), and pharmacological inhibition or genetic perturbation of CK2 reduces biochemical and transcriptional programs driving inflammation in glia. NF-κB is a major transcriptional driver of inflammation. CK2 modulates NF-κB activity via phosphorylation of NF-κB S529 and IκBα S32 and downregulates NF-κB transcriptional signatures. CK2 activity is upregulated in AD, Huntington's disease (HD), and Parkinson's disease (PD) patients. In AD patient-derived astrocytes, this correlates with higher secretion of alarmin HMGB1, which can be blocked by CK2 inhibition. AD patient-derived neurons also have overactivated CK2 signaling and mitochondrial dysfunction. CK2 thus may play a dual pathogenic role in certain neuroinflammatory disease, such as AD.

Some embodiments of the disclosed compounds are inhibitors of CK2 enzyme activity. Inhibiting CK2 kinase activity reduces or blocks inflammatory signaling and or improves mitochondrial phenotypes, for example a reduction of at least 50%, at least 60%, at least 70%, at least 80%, at least 90%, at least 95%, at least 98%, at least 99%, or even 100%, for example as compared to such activity in the absence of a compound according to formulas I-IV. In any of the foregoing or following embodiments, the compound may be a targeted degrader where R is -L-Q, and inhibiting CK2 activity further comprises degrading the CK2 enzyme. Exemplary methods of measuring CK2 kinase activity are provided herein.

Embodiments of a method of inhibiting CK2 activity include contacting a cell that expresses CK2 enzyme with an effective amount of one or more compounds as disclosed herein, thereby inhibiting activity of the CK2 enzyme. Contacting may be performed in vitro, in vivo, or ex vivo. In some embodiments, the cell is an astrocyte, a microglia, a neuron, a white blood cell, an adipocyte, a myocyte, or an epithelial cell. The white blood cell may be a granulocyte (neutrophil, eosinophil, or basophil), a phagocyte (dendritic cell, monocyte, macrophage), a lymphocyte (T cell, B cell, natural killer (NK) cell)).

In any of the foregoing or following embodiments, inhibiting activity of the CK2 enzyme may inhibit phosphorylation of one or more biomarkers, increase mitophagy, decrease mitochondrial fission, increase mitochondrial function, or any combination thereof. In some embodiments, the one or more biomarkers are HMGB1, S100A9, SORCS1, IFI16, ILF2, IFNL1, ARFGAP1, RL6IP4, DTD1, SQSTM1, FERMT2, HDLBP, MAP4K4, NAV1, PNPLA6, SMC3, TMX2, IMMT, NF-KB, IκBa, FUNDC1, CK2 (pY255), or any combination thereof.

In any of the foregoing or following embodiments, contacting the cell with the compound may include administering a therapeutically effective amount of the compound, or a therapeutically effective amount of a pharmaceutical composition comprising the compound, to a subject. The subject may be an animal, such as a mammal. In some examples, the subject is a human. In some embodiments, the subject has a disease or condition characterized at least in part by dysregulated CK2 activity. The subject may be identified as having such a disease or condition by any suitable means as understood by a person skilled in the art, such as a physician or diagnostician. Suitable means for identifying the subject as having such a disease or condition may include laboratory tests, imaging, physical evaluation, and the like.

In some embodiments, administering the therapeutically effective amount of the compound or the therapeutically effective amount of the pharmaceutical composition comprising the compound to the subject ameliorates at least one sign or symptom of the disease or condition. “Ameliorate” means that at least one sign or symptom is reduced. In certain embodiments, the sign or symptom may be eliminated. Thus, administration of the compound may reduce severity of the disease or condition, slow progression of the disease or condition, or treat the disease or condition. In some embodiments, the compound may be administered on a prophylactic basis to prevent a disease or condition characterized at least in part by dysregulated CK2 activity.

In some embodiments, the disease or condition is characterized at least in part by inflammation, and administering the therapeutically effective amount of the compound or the therapeutically effective amount of the pharmaceutical composition comprising the compound to the subject may reduce the inflammation. In certain embodiments, the inflammation is neuroinflammation.

In any of the foregoing or following embodiments, the disease or condition may be cancer (e.g., cancers with mutated CSNK2A1 genotype or upregulated CK2 levels, such as cancers of the breast, lung, colon, and prostate), cardiac hypertrophy, cystic fibrosis, a neurodegenerative disease, bipolar disorder, depression, a viral infection, obesity, diabetes mellitus, atherosclerosis, epilepsy, or any combination thereof. Exemplary neurodegenerative diseases include, but are not limited to, Parkinson's disease, Huntington's disease, Alzheimer's disease, multiple sclerosis, and amyotrophic lateral sclerosis. Exemplary viral infections include, but are not limited to, coronavirus infections, such as SARS-CoV-2 infections. Advantageously, some embodiments of the disclosed compounds may be dual targeting compounds, e.g., reducing neuroinflammation and mitochondrial defects in central nervous system diseases.

In some examples, the disease or condition is a viral infection, such as a positive-strand RNA viral infection or negative-strand RNA viral infection. Exemplary positive-strand RNA viral infection includes, but are not limited to, infection with one or more of: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain and SARS-CoV-2). Exemplary negative-strand RNA viral infections includes, but are not limited to infection with one or more of: Orthomyxyoviruses (such as the influenza virus), Rhabdoviruses (such as Rabies virus), and Paramyxoviruses (examples of which include measles virus, respiratory syncytial virus, and parainfluenza viruses). In some examples, the disease or condition is a DNA viral infection, such as: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19). In some examples, the disease or condition is a Retrovirus infection, such as human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In some examples, the disease or condition is a cancer, such as a solid tumors such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the cancer is an adenocarcinoma, such as prostate adenocarcinoma. In some examples, the disease or condition is a liquid tumor, such as a lymphatic, white blood cell, or other type of leukemia. In a specific example, the cancer is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

The compound or pharmaceutical composition may be administered to the subject through any suitable route. In some embodiments, the compound or pharmaceutical composition is administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). In some embodiments, the compound or pharmaceutical composition is administered to the subject by injection. The therapeutically effective dosages of the agents can be provided as repeated doses within a prolonged prophylaxis or treatment regimen that will yield clinically significant results to alleviate one or more symptoms or detectable conditions associated with a targeted condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages can be determined using in vitro models. Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative embodiments, an effective amount or effective dose of the agents may simply inhibit or enhance one or more selected biological activities correlated with a disease or condition, as set forth herein, for either therapeutic or diagnostic purposes.

The actual dosages of the agents will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the agent for eliciting the desired activity or biological response in the subject. Dosage regimens can be adjusted to provide an optimum prophylactic or therapeutic response. A therapeutically effective amount is also one in which any toxic or detrimental side effects of the agent is outweighed in clinical terms by therapeutically beneficial effects. A non-limiting range for a therapeutically effective amount of a compound according to any one of formulas I-IV within the methods and formulations of the disclosure is 0.001 mg/kg body weight to 100 mg/kg body weight, such as 0.01 mg/kg body weight to 20 mg/kg body weight, 0.01 mg/kg body weight to 10 mg/kg body weight 0.05 mg/kg to 5 mg/kg body weight, or 0.1 mg/kg to 2 mg/kg body weight. Dosage can be varied by the attending clinician to maintain a desired concentration at a target site (for example, systemic circulation). Higher or lower concentrations can be selected based on the mode of delivery, for example, trans-epidermal or oral delivery versus intravenous or subcutaneous delivery. Dosage can also be adjusted based on the release rate of the administered formulation, for example, of sustained release oral versus injected particulate or transdermal delivery formulations, and so forth.

In any of the foregoing or following embodiments, the therapeutically effective amount may be administered at intervals for a period of time effective to provide a therapeutic effect, e.g., amelioration of at least one sign or symptom of a disease or condition characterized at least in part by dysregulated CK2 activity. In some embodiments, the intervals are once daily. In other embodiments, the therapeutically effective amount may be divided into two or more doses administered at intervals in a 24-hour period. In some embodiments, the effective period of time is from one day to several months, such as from one day to 12 months, three days to six months, seven days to three months, 7-30 days, or 7-14 days. In certain embodiments, where the disease or condition is chronic, the effective period of time may be even longer than 12 months, such as a period of years.

In some examples, a subject treated one or more compounds provided herein to reduce CK2 enzyme activity can receive additional treatment with other compounds, such as one or more antiviral compounds, one or more anti-inflammatory agents (such as a steroid, such as a corticosteroid), one or more chemotherapeutic agents, and one or more biologics (such as a monoclonal antibody used to treat cancer, such as one specific for PD1, EGFR (e.g., cetuximab), VEGF (e.g., bevacizumab), CTLA4, or a tumor-specific antigen such as HER2 (e.g., trastuzumab), CD52 (e.g., alemtuzumab), CD20, or CD19).

### V. Representative Embodiments

Certain representative embodiments are exemplified in the following numbered paragraphs.

1. A compound according to formula I, or a pharmaceutically acceptable salt, hydrate, stereoisomer, or tautomer thereof:

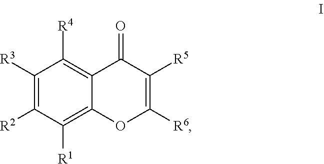

where R¹ is H, halo, —OR^{a}, substituted or unsubstituted aryl, or substituted or unsubstituted heteroaryl; R² is H or —O(CH₂)ₘR^{a} where m is an integer from 0 to 10; R³ is H, halo, —OR^{a},

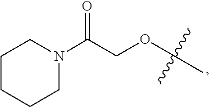 [SMILES: *OCC(=O)N1CCCCC1]

or a hydrophobic group; R⁴ and R⁵ independently are H or —OR^{a}; R^{a} is H, substituted or unsubstituted aliphatic, substituted or unsubstituted heteroaliphatic, or acyl; and R⁶ is —(CH₂)ₙ—R^{b} where n is an integer from 1-10 and R^{b} is substituted or unsubstituted heteroaliphatic or R^{b} is R¹¹, where R¹¹ is substituted or unsubstituted heteroaryl or substituted or unsubstituted aryl, or R⁶ is

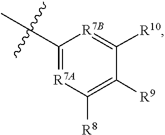

where
one of R^{7A} and R^{7B} is CH or N, and the other of R^{7A} and R^{7B} is CH, and R⁸ is H or —OR^{a}, R⁹ is —OR^{a}, —CN, —C(O)OR^{a}, or azole, and R¹⁰ is H, alkynyl, or -alkynyl-R¹¹, or R⁸/R⁹ or R⁹/R¹⁰ together with the atoms to which they are bound form a substituted or unsubstituted heteroaliphatic or heteroaryl ring, wherein if R^{7A} and R^{7B} are both CH, then (i) R⁹ is other than —OR^{a}, or (ii) R¹⁰ is heteroaryl-substituted alkynyl, or (iii) R⁹ and R¹⁰ together with the atoms to which they are bound form a substituted or unsubstituted heteroaliphatic or heteroaryl ring including at least one N atom in the ring, or (iv) R¹ is furan substituted with —C(O)OR^{a}.

2. The compound of paragraph 1, having a structure according to any one of formulas II-IV, or a pharmaceutically acceptable salt, hydrate, stereoisomer, or tautomer thereof:

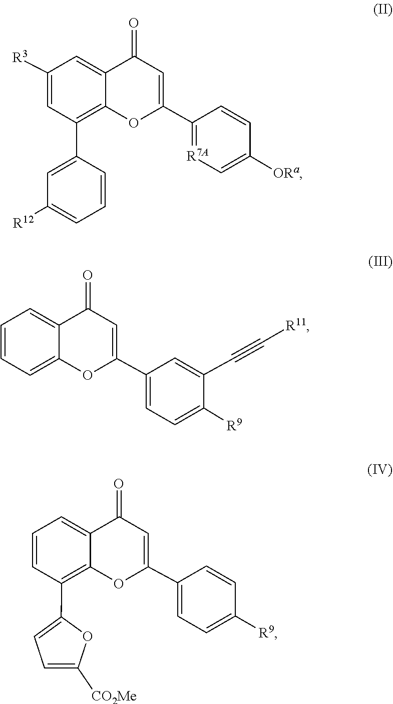

where R³ is halo or a hydrophobic group; R⁹ is —CO₂H, —CN, or —OH; R¹¹ is heteroaryl; R¹² is halo, alkynyl, or -alkynyl-R¹¹; and R^{a} is H or acyl.
3. The compound of paragraph 1 or paragraph 2, where R¹¹ is

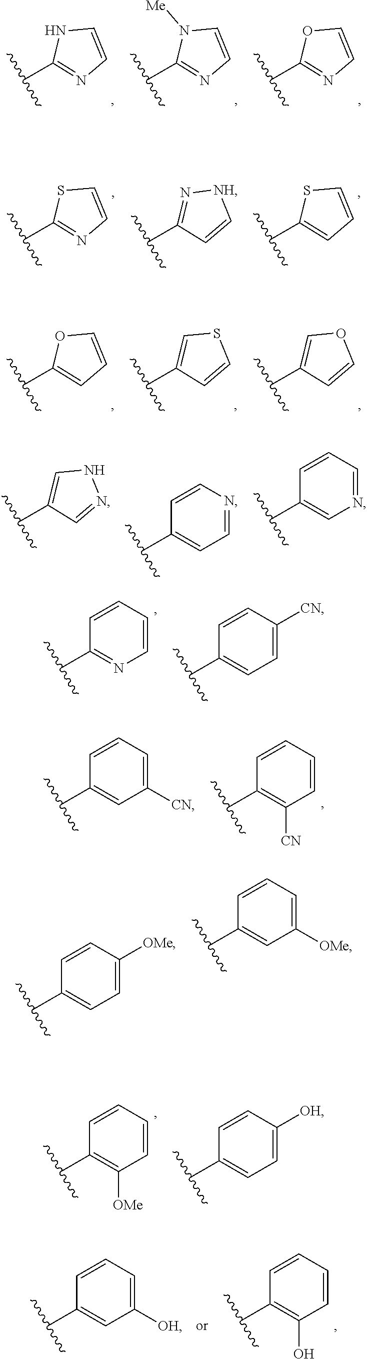

4. The compound according to any one of paragraphs 1-3, wherein: (i) R⁶ is

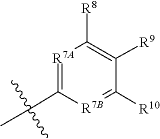

where R⁸ is H, R⁹ is —OH, —CO₂H, —CN, or —OCH₂CH═CH₂ and R¹⁰ is H or

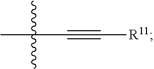

### or (ii) R⁶ is

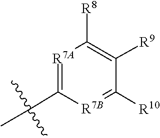

where R⁸ and R¹⁰ are H, and R⁹ is azole; or (iii) R⁶ is —(CH₂)₂—R^{b} where R^{b} is an azole, triazine, or tetrahydrofuran; or (iv) R⁶ is

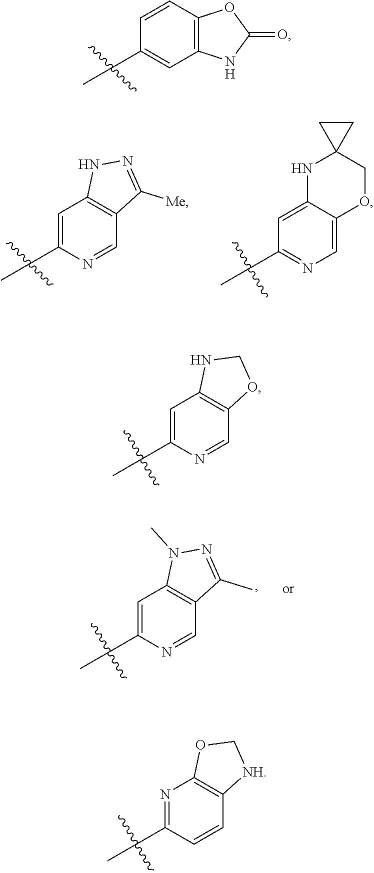 [SMILES: *c1ccc2oc(=O)[nH]c2c1 ; *c1cc2[nH]nc(C)c2cn1 ; *c1cc2c(cn1)OCC1(CC1)N2 ; *c1cc2c(cn1)OCN2 ; *c1cc2c(cn1)c(C)nn2C ; *c1ccc2c(n1)OCN2]

5. The compound according to any one of paragraphs 1-4, wherein R^{a} is H or methyl.

6. The compound according to paragraph 1, wherein: (i) R¹ and R³ are halo or methoxy, and R² and R⁴ are H; or (ii) R¹ and R³ are H, and R² and R⁴ are halo or methoxy; or (iii) R¹, R³, and R⁴ are H, and R² is methoxy; (iv) R¹-R⁴ are H; or (v) R¹, R³, and R⁴ are H, and R² is

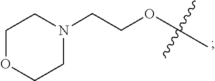 [SMILES: *OCCN1CCOCC1]

or (vi) R¹, R², and R⁴ are H, and R³ is

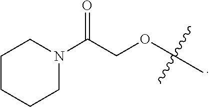 [SMILES: *OCC(=O)N1CCCCC1]

7. The compound according to paragraph 1, wherein: (i) the compound is capable of passing through the blood-brain barrier; or (ii) the compound has an aqueous solubility ≥500 μM; or (iii) both (i) and (ii).

The compound according to paragraph 1, wherein the compound is:

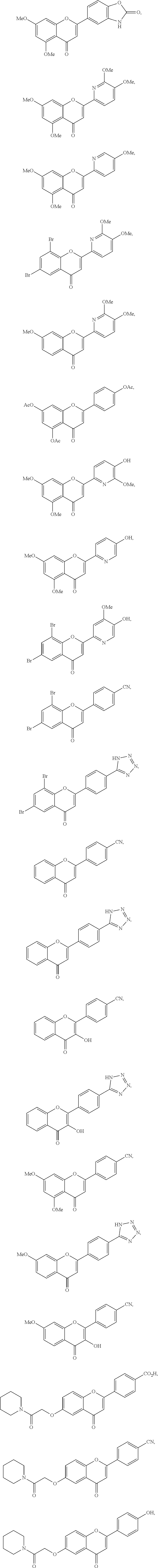

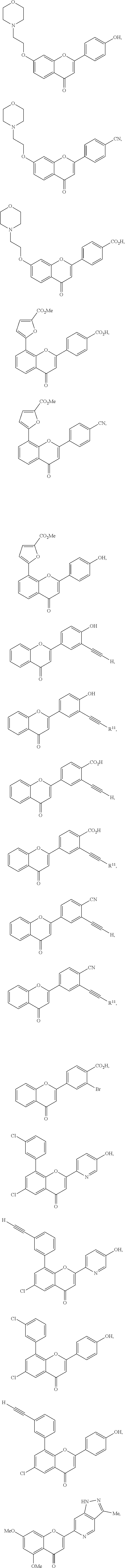

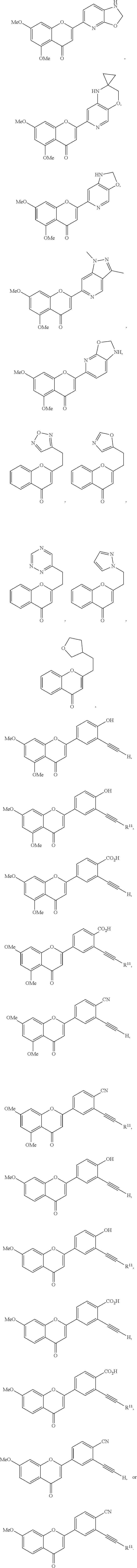

where R¹¹ is substituted or unsubstituted heteroaryl.

9. A pharmaceutical composition, comprising a compound according to any one of paragraphs 1-8 and a pharmaceutically acceptable excipient.

10. A method of inhibiting CK2 activity, comprising: contacting a cell that expresses CK2 enzyme with an effective amount of a compound according to any one of paragraphs 1-9, thereby inhibiting activity of the CK2 enzyme.

11. The method of paragraph 10, wherein the cell is an astrocyte, a microglia, a neuron, a white blood cell, an adipocyte, a myocyte, or an epithelial cell.

12. The method of paragraph 10 or paragraph 11, wherein inhibiting activity of the CK2 enzyme inhibits phosphorylation of one or more biomarkers, increases mitophagy, decreases mitochondrial fission, increases mitochondrial function, or any combination thereof.

13. The method of paragraph 12, wherein the one or more biomarkers are HMGB1, S100A9, SORCS1, IFI16, ILF2, IFNL1, ARFGAP1, RL6IP4, DTD1, SQSTM1, FERMT2, HDLBP, MAP4K4, NAV1, PNPLA6, SMC3, TMX2, IMMT, NF-KB, IκBα, FUNDC1, CK2 (pY255), or any combination thereof.

14. The method of any one of paragraphs 10-13, where contacting the cell with the compound comprises administering a therapeutically effective amount of the compound, or an amount of a pharmaceutical composition comprising the therapeutically effective amount of the compound, to a subject.

15. The method of paragraph 14, wherein the subject has a disease or condition characterized at least in part by dysregulated CK2 activity.

16. The method of paragraph 15, wherein administering the therapeutically effective amount of the compound or the amount of the pharmaceutical composition comprising the therapeutically effective amount of the compound to the subject ameliorates at least one sign or symptom of the disease or condition.

17. The method of paragraph 15 or paragraph 16, wherein the disease or condition is characterized at least in part by inflammation.

18. The method of paragraph 17, wherein the inflammation is neuroinflammation.

19. The method of paragraph 15 or paragraph 16, wherein the disease or condition is cancer, cardiac hypertrophy, cystic fibrosis, a neurodegenerative disease, bipolar disorder, depression, a viral infection, obesity, diabetes mellitus, atherosclerosis, or any combination thereof.

20. The method of paragraph 19, wherein the neurodegenerative disease is Parkinson's disease, Alzheimer's disease, multiple sclerosis, or amyotrophic lateral sclerosis.

21. The method of paragraph 19, wherein the viral infection is a SARS-CoV-2 infection.

22. The method of any one of paragraphs 13-21, wherein administering is performed orally or parenterally.

### VI. Examples

### Methods:

Postmortem samples. Fresh frozen postmortem tissue from the substantia nigra from PD (n =6) or age-matched controls (n=6) was acquired from the NIH Neurobiobank.

Cell culture. Primary fetal human cerebellar astrocytes (HCA, ScienCell) were cultured in Astrocyte Medium (AM, ScienCell). hESC and hiPSC cell lines were maintained on Matrigel (Cultrex)-coated plates in TeSR medium (made in-house by the Salk Stem Cell Core), fed daily and passaged with dispase every 5-7 days (Gibco). All subjects provided written informed consent and all procedures were approved by local human subjects committees. Mature hES-derived astrocytes were cultured in DMEM/F12 Glutamax (Thermo Fisher Scientific) supplemented with N2 and B27 (Thermo Fisher Scientific) and 10% fetal bovine serum (FBS, Biowest). Mature iPSC-derived microglia were cultured in DMEM/F12 Glutamax serum-free media (see below) supplemented with TGF-b1, IL-34, and M-CSF. All cell lines were maintained in a humidified incubator (5% CO₂) at 37° C. and were routinely tested for mycoplasma. THP-1 NFKB-Luciferase reporter cells (InvivoGen) were grown in RPMI 1640 with 10% FBS, Glutamax, 25 mM HEPES pH=7.5, and penicillin/streptomycin (P/S). Vero-E6 cells (kind gift from Sandra Leibel) were grown in DMEM plus 10% FBS and P/S.

Animals. All animal procedures were approved by the Institutional Animal Care and Use Committee of The Salk Institute for Biological Studies. Wildtype male C57BL/6 mice (2-3 months old) were housed under standard 12-h light/dark cycles with free access to food and water. IN2.2 or vehicle (3% hydroxypropylmethylcellulose, HPMC) was injected as a single dose (25.6 mg/kg) i.p. mice (n=6 and n=3 controls). Blood was collected at t=0, 0.5, 1, 2, 4, 8, 24 via retro-orbital bleeding and allowed to clot at room temperature to separate serum and clarified. At the final timepoint, mice were anesthetized with ketamine and tissues were harvested. Drug concentrations in serum and tissues were determined by LC/MS-MS. Target engagement in the brain was determined in brain homogenates from treated animals using the Cyclex CK2 ELISA kit. JKT56/IN3.2 or vehicle (2% HPMC) was injected as a single dose (20 mg/kg) i.p. in mice (n=6). Blood and tissues were collected and processed by LC-MS/MS as described above for IN2.2. Tolerability was assessed mice (mean±s.d., n=5) by injecting with JKT56/IN3.2 daily at 20 mg/kg and monitoring closely for any signs of toxicity based on body weight changes and behavior/appearance (e.g. signs of distress, rough coat, squinty eyes, hunched posture).

Compounds. Natural products were sourced as follows: chrysin (Sigma Aldrich), TMF, dTMF, chrysoeriol, homoeryodictyol (Indofine Chemical). Apigenin was a kind gift from the Mars Corporation. Naringenin was a kind gift from James LeClair. Compounds were stored at −20° C. as 20 mM stock solution aliquots in DMSO.

THP-1 NF-κB-Luciferase reporter assay. Dose response curves were generated for several known compounds using THP-1 NF-κB-Luciferase reporter cells. The THP-1 cells were differentiated for 3 days with 10 ng/mL PMA into monocytes. Then, cells were treated+/−LPS and 8 concentrations of each compound or vehicle for 5 hours, after which media was transferred to a 384-well white plate (Corning), luciferase substrate was added, and luminescence was read using a Promega GloMax Discover microplate reader.

Differentiation of astrocytes from hES and hiPSC. H1 human embryonic stem cells (hESC, WiCell Research Institute) or AD and control iPSCs (UCI ADRC iPS Cell Bank) were cultured on Matrigel-coated plates in mTeSR medium and differentiated into glial progenitor cells (GPC) and mature astrocytes as previously described (Santos et al., Stem Cell Reports 2017, 8:1757-1769). First, embryoid bodies were prepared by mechanical dissociation of H1 cultures with 1 mg/mL collagenase IV (Gibco), plated onto ultra-low attachment plates (Corning) in TeSR medium with 10 μM Y-27632 (ROCK inhibitor, StemCell Tech), and incubated overnight with rocking. For differentiation of GPCs from embryoid bodies, media was changed to AM supplemented with 500 ng/mL Noggin (R&D Systems) and 10 ng/mL PDGFAA (Peprotech) for 2 weeks and then Noggin was withdrawn for another week. The embryoid bodies were dissociated with papain (Papain dissociation system, Worthington) and the GPCs were cultured and expanded in 10 mg/mL poly-L-ornithine (Sigma)/1 mg/mL laminin (Invitrogen)-coated plates in AM supplemented with 20 ng/mL fibroblast growth factor 2 (FGF-2, Joint Protein Central) and 20 ng/mL epidermal growth factor (EGF, Humanzyme). Astrocytes were differentiated from low-confluent GPC cultures in DMEM/F12 Glutamax supplemented with N2 and B27 and 10% FBS. After 2 weeks of differentiation, the cells were transferred to non-coated plates for another 2 weeks of maturation.

Differentiation of microglia-like cells from iPSCs. H1 hESC and EC11 or Clue4-7 (Schafer et al., Nature Neuroscience 2019, 1-20, doi:10.1038/s41593-018-0295-x) iPSC were cultured on Matrigel-coated plates in mTeSR medium and differentiated into mature microglia-like cells (iMGL) via an induced hematopoietic progenitor cell (iHPC) intermediate, as previously described (Abud et al., Neuron 2017, 278-293.e9). First, confluent stem cell cultures were dissociated into single-cell suspensions with TrypLE (Gibco) and plated onto untreated tissue-culture plates in TeSR with 10 μM Y-27632. The next day, media was changed to iHPC basal medium supplemented with FGF2 50 ng/ml, BMP4 50 ng/ml (Peprotech), Activin-A 12.5 ng/ml (Proteintech), 1 μM Y-27632, and 2 mM LiCl (Sigma), and placed in a hypoxic incubator (5% 02, 5% CO₂) for 4 days. iHPC basal medium was composed of 50% DMEM/F12, 50% IMDM, 2% ITS-X Insulin-Transferrin-Selenium-Ethanolamine (Thermo-Fisher Scientific), L-ascorbic acid 2-phosphate magnesium (64 μg/ml; Sigma), monothioglycerol (400 μM), PVA (10 μg/ml; Sigma), Glutamax (1×), chemically-defined lipid concentrate (1×), non-essential amino acids (NEAA; 1×), and 1% Penicillin/Streptomycin. Next, cells were cultured in normoxic conditions for another 6-16 days with fresh media supplemented with FGF2 50 ng/ml, VEGF 50 ng/mL (Proteintech), TPO 50 ng/ml (Proteintech), SCF 10 ng/ml (Proteintech), IL-6 50 ng/ml (Proteintech), and IL-3 10 ng/mL (Proteintech) added every 2 days. The resulting iHPCs were collected and sorted for CD43+ staining by FACS. Next, the pure iHPCs were differentiated into microglia-like cells by a 25-day maturation in iMGL medium (50% DMEM/F12, 50% IMDM, 2% ITS-G Insulin-Transferrin-Selenium (Thermo-Fisher Scientific), 2% B27, 0.5% N2, 200 μM monothioglycerol, 5 μg/mL insulin (Sigma), Glutamax (1×), NEAA (1×), 1% Penicillin/Streptomycin (ScienCell) supplemented with cytokines. For the first 22 days, cytokines M-CSF 25 ng/ml, IL-34 100 ng/ml, and TGFb-1 50 ng/ml (Proteintech) were added. Following a 3 day maturation with the addition of CD200 100 ng/ml and CX3CL1 100 ng/ml, the iMGLs were FACS-sorted using a 6-antibody panel. CD45+, CD11b+, CD64+, CD14+, CX3CR1+ and HLA-DR+ cells were plated onto 96-, 24- or 6-well Primaria plates (Corning) for downstream bead phagocytosis and activation assays.

Flow cytometry. For each well of HCA in a 6-well plate, 2 mL of fresh media with GolgiPLUG (BD #555029, 1:1000) and GolgiSTOP (BD #554724, 1:1000) with 20 μM compound or vehicle (DMSO) and IL1-β 10 ng/mL or DPBS was added. The cells were incubated for 5 h at 37° C. Cells were collected with TrypLE and plated into a 96-well V-bottom plate for staining. The plate was centrifuged for 5 min, 1300 rpm, 4° C. to pellet cells, which were subsequently resuspended in 100 μl DPBS with 1 μl Zombie Violet (Biolegend #423113) and 5 μl Human Trustain FC block (Biolegend #422302) per well for 15 min at room temperature in the dark. The plate was centrifuged, supernatant was aspirated, and the cells were washed with 100 μl/well DPBS. Cells were fixed with 100 μl/well of Cytofix/Cytoperm (BD) for 20 min at 4° C. The plate was centrifuged, supernatant was aspirated, and cells were resuspended in 100 μl Permwash (BD) and incubated for 15 min at 4° C. The plate was centrifuged, supernatant was aspirated, and cells were incubated with 100 μl/well of Ab mix or isotype mix in Permwash (see below) for 20 min at 4° C., followed by centrifugation and 2× PermWash washes. The cells were resuspended in 100 ul DPBS and transferred to FACS tubes containing 200 ul of PBS (total volume per tube 300 μl) for analysis on FACS Analyzers (LSRII or Fortessa).

| Antigen | Fluorochrome | Volume | Conc. | isotype | clone | catalog # | Company |
|---|---|---|---|---|---|---|---|
| IL-8 | PercP | 5 μL | 100 μg/ml | Mouse | BH0814 | 514606 | Biolegend |
| | | | | IgG2b | | | |
| TNF-a | Pe-Cy7 | 5 μL | 200 μg/ml | Mouse | MAb11 | 502930 | Biolegend |
| | | | | IgG1, k | | | |
| IL-6 | APC | 5 μL | 50 μg/ml | Rat | MQ2- | 501112 | Biolegend |
| | | | | IgG1, k | 13A5 | | |
| MCP-1 | PE | 3 μL | 200 μg/ml | Armenian | 2H5 | 505904 | Biolegend |
| (CCL2) | | | | Hamster IgG | | | |

FACS sorting. For FACS sorting of iHPC and iMGL, cells were collected and filtered PGP-61,T2 through a 70 μm mesh cell strainer attached to a 50 ml conical tube to remove large clumps and centrifuge at 1000 rpm for 3 min. The pellet was washed with 3 mL of cold sterile filtered FACS buffer (1×DPBS, 2% BSA, and 0.05 mM EDTA pH 8.0) and transferred to a 15 ml conical tube centrifuge. They were resuspended in 100-300 μl of FACS buffer, 5 μl/100 μl of Trustain was added, and the cells were incubated at RT for 5 min. 5 μl of cells were removed from the sample and transferred to a new conical tube, brought to 100 μl of FACS buffer and 5 μl of corresponding isotype control antibody (see below) was added along with 1 μl of Zombie Violet live/dead stain. For each sample, 5 μl of each antibody (see below) and 1 μl-3 μl of Zombie Violet live/dead stain was added. Each sample and the isotype control was incubated on ice in the dark for 20 min. After staining, the samples were washed once with 1 ml of cold FACS buffer, centrifuged and resuspended in 100-300 μl FACS buffer. The sample and the isotype control were transferred into separate FACS tubes with a 40 μm filter attached. For iHPC, all live and CD43+cells were sorted into cold basal iHPC differentiation medium including Pen/Strep. For iMGL, all live and CD45+, CD11b+, CD64+, CD14+, CX3CR1+ and HLA-DR+ cells were sorted into cold basal iMGL differentiation medium.

| Color | Antibody |
|---|---|
| APC | CD64 |
| PerCP-Cy5.5 | CX3CR1 |
| 488/FITC | CD14 |
| PE | CD11b |
| PE-Cy7 | HLA-DR |
| APC-Cy7 | CD45 |
| FITC | CD43 |
| FITC | Isotype Ct |
| APC | Isotype Ct |
| PerCP-Cy5.5 | Isotype Ct |
| FITC | Isotype Ct |
| PE | Isotype Ct |
| PE-Cy7 | Isotype Ct |
| APC-Cy7 | Isotype Ct |

Cytokine release assay. Microglia were treated in 6-well plates with fresh iMGL media and either vehicle alone (DMSO) or 200 ng/mL lipopolysaccharide (LPS, Sigma) with 20 μM CHR or vehicle for 5 hours in duplicate. Subsequently, media cleared of cells and debris was collected, snap-frozen and stored at -80 C until processing with the Human Cytokine Array kit (R&D Systems, Catalog #ARY005). Briefly, media was diluted in Array Buffer 4 and incubated with a cocktail of biotinylated detection antibodies for 1 h at room temperature with rocking. The resulting immunocomplexes were incubated with nitrocellulose membranes spotted with capture antibodies overnight at 4 C (see below for full list of targets). The membranes were washed 4× with Wash Buffer, then incubated with IRDye 800CW Streptavidin for 30 minutes at room temperature (1:2000 in Array Buffer 5, LI-COR #926-32230). The membranes were washed 4× and imaged with an Odyssey CLx instrument. Intensity for each spot was quantified with ImageStudio. Technical duplicate values for each target were averaged, and we determined differential cytokine release by microglia exposed to LPS+vehicle versus vehicle only (n=2 biological replicates, two-tailed t-tests, P<0.05). Then, we determined whether the release of those cytokines was significantly affected by CHR treatment (n=2 biological replicates, two-tailed t-tests, P<0.05).

| | | |
|---|---|---|
| C5a | IL-4 | IL-32 alpha |
| CD40 ligand | IL-5 | CXCL10/IP-10 |
| G-CSF | IL-6 | CXCL11/I-TAC |
| GM-CSF | IL-8 | CCL2/MCP-1 |
| CXCL1/GRO alpha | IL-10 | MIF |
| CCL1/I-309 | IL-12 p70 | CCL3/MIP-1 alpha |
| ICAM-1 | IL-13 | CCL4/MIP-1 beta |
| IFN-gamma | IL-16 | CCL5/RANTES |
| IL-1 alpha | IL-17 | CXCL12/SDF-1 |
| IL-1 beta | IL-17E | Serpin E1/PAI-1 |
| IL-1ra | IL-23 | TNF-alpha |
| IL-2 | IL-27 | TREM-1 |

Phagocytosis assay. Microglia were pre-treated in 12-well plates with fresh iMGL media with vehicle (DMSO), 20 μM CHR, or 20 μg/mL cytochalasin D for 1 h at 37 C (2-3 biological replicates). Then, 0.5 μl of pH rodo E. coli-FITC beads (Thermo Fisher, P35366) was added per well with gentle mixing and the cells were incubated for another 4 hours. pHrodo beads only fluoresce when they are internalized in cells. Cells were then collected by scraping, washed with FACS buffer, and counter-stained with Zombie Violet to assess viability. Live cells were analyzed on the FACS Canto instrument for geometric mean fluorescence on the FITC channel. The background FITC fluorescence value (pHrodo beads only) was subtracted from each mean, and fold-change was computed across experimental groups.

Western blotting. For drug treatment assays, cells were treated with vehicle (DMSO) or 20 μM compound and stimulated with IL1-B 10 ng/mL for 1 h, 6 h, or 24 h. Cells were dissociated with 1:1 Accutase:papain for 3 min at rt, pelleted for 5 min at 800 g, and washed twice with cold DPBS. Pellets were lysed with RIPA buffer (Sigma R0278) supplemented with Halt protease inhibitor cocktail (Thermo Fisher 78445) and clarified by centrifugation (16,000 g, 10 min, 4° C.). Protein concentration was determined by BCA assay (Thermo Fisher). Proteins were denatured in 1×LDS Buffer and 2.5% beta-mercaptoethanol (X) for 10 min at 70° C., separated on Bolt 4-12% Bis-Tris Plus polyacrylamide gels (Thermo Fisher), transferred to PVDF membranes using the iBlot 2 Dry Blotting System, and blocked with 0.1% casein-PBS (Bio-Rad) for 1 h at room temperature. The blots were incubated with primary antibodies overnight at 4° C. in PBS containing 0.1% casein and 0.2% Tween-20. Antibodies used: rabbit anti-CK2a1 (Bethyl #, 1:5000), rabbit anti-CK2a2 (Bethyl #, 1:5000), mouse anti-GAPDH (Fitzgerald #10-1501, 1:20,000), mouse anti-NF-KB (CST #6956S, 1:1000), pNF-KB S529 (TFS #14-9864-82, 1:100), LC3B (CST 2775, 1:1000), ACO2 (CST 6922, 1:1000), rabbit HMGB1 (Novus #NB1002322, 1:1000), mouse anti-IκBα (CST #4814S, 1:1000), mouse anti-IκBa pS32 (SC #8404, 1:500), HA-tag (Bethyl #A190-108A, 1:20000). The membranes were washed 5× with 0.1% PBS-Tween20 (PBST) and incubated with secondary antibodies (1:20,000) for 1 h at rt in PBS with 0.1% casein, 0.2% Tween20, and 0.1% SDS. Secondary antibodies: goat anti-mouse IgG IRDye 800CW #925-32210, goat anti-rabbit mouse IgG IRDye 800CW #925-32211, goat anti-mouse IgG IRDye 680RD #926-68070, or goat anti-mouse IgG IRDye 680RD #926-68071. The blots were then washed 4× with PBST and 1× with DPBS. The blots were imaged using the Odyssey CLx imaging system and blots were analyzed on the ImageStudio software (LI-COR).

Immunotluorescence. Primary human astrocytes (ScienCell) were plated on glass slides (EMD Millipore PEZGS0896) and treated with CHR, CX-4945/silmitasertib, or DMSO and stimulated with IL1-B for 1 hour, 5 hours or 25 hours. After treatment, cells were fixed with 4% paraformaldehyde solution for 20 min at room temperature and washed 3× with DPBS for 10 min each. The cells were permeabilized using 5% horse serum and 10% Triton X in DPBS for 15 min at room temperature and blocked with 5% horse serum in PBS for 30 min at room temperature. Cells were incubated with primary antibodies in blocking buffer either overnight at 4° C. or for 2 hours at room temperature. After incubation, cells were washed 3× with PBS, blocked using 5% horse serum in PBS for 30 min at room temperature, and incubated with Cy3 red-labeled or AF488 green-labeled secondary antibodies diluted in blocking buffer for 1 hour at room temperature. The cells were washed once with PBS for 10 min, then counter-stained with DAPI. The cells were washed 3× with PBS and mounted with glass coverslips and Immu-Mount solution. Images were obtained with the Zeiss fluorescent microscope. The following primary antibodies were used: rabbit anti-NF-κB (1:400; CST), mouse anti-phospho NF-κB S529 (20 ug/ml; Thermo Fisher), rabbit anti-CK2A1 (1:500; Bethyl), mouse anti-IKb alpha (1:400; CST), mouse anti-phospho IKb alpha (1:50; Santa Cruz), rabbit anti-phospho CK2A1 (1:25; Thermo Fisher), rabbit anti-phospho Cdc37 (1:50; Novus), mouse anti-phospho NF-κB S529 (1:25; R&D), rabbit anti-Cdc37 (1:100; CST), mouse anti-SQSTM1 (1:50; Santa Cruz), anti rabbit-phospho NF-κB S536 (1:1600; CST), anti rabbit-Akt (pan) (1:400; CST), anti mouse-phospho Akt S473 (1:500; Sigma).

Activation assays and ddPCR. HCA or H1-derived astrocytes were treated with vehicle (DMSO) or 20 μM compound and stimulated with IL1-B 10 ng/mL for 5 h. Cells were scraped into RNA-Bee and total RNA was purified using the Direct-zol RNA kit (Zymo Research). RNA concentration was measured using Nanodrop and RNA was reverse transcribed into cDNA using the High Capacity cDNA Reverse Transcriptase Kit (Applied Biosystems). Reaction mixes consisting of TaqMan FAM probe (IL6, IL8, CSNK2A1, CSNK2A2), control TaqMan ACTB-VIC probe (TFS 4326315E), ddPCR Supermix for Probes (Bio-Rad 186-3024), and cDNA were formed into oil droplets using the QX200 Droplet Generator. After amplification (Bio-Rad C1000 Touch Thermal Cycler) according to manufacturer's instructions, the plate was read in the QX200 Droplet Reader.

Cloning and lentiviral transduction. pBOB-CAG-CK2a1-WT, pBOB-CAG-CK2a2-WT, pBOB-CAG-CK2a1-K68M, pBOB-CAG-CK2a1-K69M plasmids were generated by cloning HA-tagged CK2 inserts from parent plasmids (Litchfield lab, Addgene 27086, 27090, 27089, 27087) into the pBOB backbone (Addgene #12337). Plasmids were transformed into TOP10 competent cells (Thermo Fisher). H1-GPCs in 6-well plates were transduced with lentiviral particles (˜10⁸ particles/mL, Salk Virus Core) by incubating for 3 days. Media was changed and cells were differentiated into mature astrocytes as described above after checking for HA-CK2 expression by Western blotting. Astrocytes were processed as described under “Activation assays, ddPCR”.

siRNA knockdown. HCAs were nucleofected with siRNAs against CSNK2A1 (Ambion s2888), CSNK2A2 (Ambion s7501), or scrambled control (Ambion 4390843) using the Amaxa Nucleofector II (Program T-019). After 48 hours, knockdown efficiency was assessed by ddPCR and cells were replated to assess inflammatory response to IL1-β as described under “Activation assays, ddPCR”. Results shown represent a single electroporation.

Thermal shift assay and TPP. Experiments were performed as previously described (Franken et al., Nat Protoc 2015, 10:1567-1593; Reinhard et al., Nat Meth 2015, 12:1129-1131). 45 million H1-derived astrocytes were activated with 10 ng/mL IL1-β for 6 h, dissociated with 1:1 Accutase/papain (3 min rt), and centrifuged at 1,800 rpm for 2 min at 4 C. The pellet was washed twice with 10 ml cold DPBS, lysed in 2.25 mL DPBS and 0.4% v/v NP-40 by freeze-thawing 3×, and clarified by centrifugation (20,000 g, 30 min, 4° C.). Protein concentration was determined by BCA assay (Bio-Rad), and lysates were diluted to 2 mg/mL. First, compound or vehicle was added (10 μl DMSO, 10 μl of 20 mM TMF, or 10 μl of 20 mM CHR) to individual tubes, then 0.99 mL lysate was added to each tube and vortexed briefly. Final concentrations: 1% vehicle, 200 μM TMF, 200 μM CHR. Reactions were incubated at rt for 30 min, after which each treated extract was divided into 10 aliquots of 95 μl in PCR strip tubes and stored on ice. Each group of treated samples was heated for 3 min at predesignated temperatures (3×10 temperatures), then kept for 3 min at rt before ultracentrifugation (20 min at 4° C., 100,000 g) in 200 μl polycarbonate tubes (Beckman Coulter #343775). Designated temperatures were 37, 41.1, 43.6, 46.9, 50, 53.7, 56, 59.5, 63, and 67° C. A small aliquot (5 μl) of supernatant was saved for Western blotting, and the rest was snap frozen at −80 C and submitted for LC-MS/MS. For isothermal dose-response experiments, CHR was added to individual extracts at concentrations in a 3-fold dilution series ranging from 200 μM-0.1 μM, including one vehicle control, and the extracts were heated at 60° C. and processed as described above.

TPP analysis. MS data was analyzed using the TR workflow of the R package TPP (version 3.10.0) as previously described (Franken et al., Nat Protoc 2015, 10:1567-1593). The package performs protein quantity normalization, fits melting curves, determines melting points, and identifies proteins that have a significant shift in thermal stability compared with controls. We used data from two independent TPP experiments to quantify significant thermal shifts between CHR and vehicle and TMF and vehicle. The algorithm was run with minor changes to default parameters (filtering requirements changed to include peptides with spectral counts ≥2 and fold-change thresholds from 0-1.5). Briefly, data for each protein and condition underwent curve-fitting analysis and significance of thermal shift was calculated only for proteins with R²>0.8 and a plateau of <0.3 for the vehicle curve. Proteins that met all 4 of the following benchmarks for significance and had spectral counts ≥3 were considered “hits”: (a) P values for the two replicate experiments were <0.05 and <0.2, respectively. (b) The compound-vehicle melting point shifts in the two independent experiments had the same direction. (c) Each compound-vehicle ΔTₘ was greater than the ΔTₘ between the two vehicle controls. (d) The minimum curve slope in each experiment was <−0.06.). Network analysis and visualization of each set of hits was performed with the GeneMania app within Cytoscape (v. 3.5.1).

Native kinase capture. Experiments were performed as previously described (Patricelli et al., Chemistry & Biology 2011, 18:699-710). 45 million H1-derived astrocytes were activated with 10 ng/mL IL1-β for 6 h, dissociated with 1:1 Accutase/papain (3 min rt), and centrifuged at 1,800 rpm for 2 min at 4 C. The pellet was washed twice with 10 ml cold DPBS, lysed in 2.25 mL DPBS by repeated freeze-thawing, sonicated (2×10 s pulses with a 30 s break, 4° C.) and clarified by centrifugation (20,000 g, 10 min, 4° C.). The supernatant was desalted through a column (732-2010, Biorad) and then eluted with cold kinase buffer (20 mM HEPES, pH 7.4, 150 mM NaCl, 0.5% Triton X-100, with Halt Protease and Phosphatase inhibitor cocktail). For each treatment, 475 μl of the lysate (2 mg/mL) was pre-incubated with 10 μl MnCl₂ (1 M) and 5 μl compound to the desired concentration at room temperature for 30 min. Uninhibited kinases were captured with 10 μl ActivX desthiobiotin-ATP probe (0.25 mM; 88311, Pierce) at room temperature for 10 min. Samples were mixed with 500 μl urea (8 M; 818710, Millipore) and 50 μl streptavidin agarose (20359, Thermo) for 60 min at room temperature on a nutator. Beads were washed twice with a 1:1 mixture of kinase buffer and 8 M urea, and collected by centrifugation (1,000 g, 1 min). Proteins were eluted from the beads with 100 μl 2×LDS sample buffer (NP0007, Life) at 95° C. for 10 min. Samples were analysed by standard immunoblotting. Experiment was performed twice.

Immunoprecipitation. Cells were treated. Cells were dissociated with Accutase and centrifuged at 1,800 rpm for 5 min at 4 C. The pellet was washed twice with cold DPBS, lysed in IP Buffer (20 mM HEPES 7.4 pH, 150 mM NaCl, 1% Triton-X, 1 mM EDTA, 1 mM EGTA plus Halt Protease and Phosphase Inhibitor Cocktail) with sonication (6×10 s pulses with 10 s breaks, 4° C.) and clarified by centrifugation (20,000 g, 10 min, 4° C.). Lysates were precleared with Protein A/G Magnetic Beads (Pierce) for 20 min at rt. Novus NF-KB antibody (R polyclonal). Proteins were eluted in 4×LDS/BME at room temperature for 10 minutes, then denatured at 70° C. for 10 minutes and run on NuPAGE gels at 1×LDS. Use anti-rabbit LC IgG only secondary for detection. antibodies and secondaries used.

Analysis of Aging, Dementia, and TBI RNA-seq data and UPP Proteomics data. Normalized RNA-seq data (as z-scores) and sample metadata for CSNK2A1, CSNK2A2, CSNK2A3 and CSNK2B expression in the parietal cortex was downloaded from https://aging.brain-map.org/download/index. Data was filtered to retain only samples from patients who had never experienced TBI and who were classified as having “Dementia” (n=21) or “No Dementia” (n=27), and individual tow-tailed t-tests were conducted to determine statistically significant differences; only CSNK2A2 exhibited differential expression between dementia and controls. For the UPP Proteomics study, normalized and batch-corrected 10 g2(abundance) data for CSNK2A1, CSNK2A2, CSNK2A3 and CSNK2B was downloaded from 10.7303/syn17009177. Data was filtered to retain only samples from patients with AD (n=44) or age-matched controls (n=48), and one-way ANOVA with Sidak's post-hoc test was conducted to determine statistically significant differences; only CSNK2A2 exhibited differential expression between AD and controls.

Kinase assays. In vitro kinase assays were run using the Cysteine-Sox-Kinase Sensor AQT0696, 96 assay Kit (AssayQuant Tech, CSKS-AQT0696). Compound stocks (1.25×) at concentrations in a 2-fold dilution series were prepared in kinase reaction mixes and added in duplicate to a 384-well white-bottom NBS plates (Corning X) at 20 μL/well. Then, 5 μL active recombinant human CK2a1 kinase (Thermo Fisher PV3248, 5× in Kinase Dilution Buffer) was added to each well, quickly mixed by rotation, and incubated for 5 h at 30° C., with fluorescence readings taken every 3 min (excitation λ=365 nm, emission λ=495-505 nm) on a Promega GloMax Discover Microplate Reader. The final reaction conditions contained 10 μM AQT0696 Csox peptide, 1 mM ATP, 54 mM HEPES pH 7.5, 1.2 mM DTT, 0.012% Brij-35, 0.52 mM EGTA, 1% glycerol, 0.2 mg/mL BSA, 10 mM MgCl₂, and 20 nM CK2a1. Slopes corresponding to linear reaction rates were obtained for each compound concentration (RFU/sec), normalized to DMSO controls, and IC₅₀ values were determined by applying the dose response non-linear regression model in GraphPad Prism. For kinase assays using postmortem brain tissue lysates, brain tissues were homogenized at 4° C. using a Bullet Blender (Next Advance) in kinase buffer (20 mM HEPES 7.4 pH, 150 mM NaCl, 1% Triton-X, 1 mM EDTA, 1 mM EGTA plus Halt Protease and Phosphase Inhibitor Cocktail). After sonication and clarification, protein concentration was measured by BCA Assay. CK2 activity was measured as described above, except lysates were used instead of purified kinase at 2 μL/well (˜0.3-0.4 ug protein per reaction). Reactions occurred too fast to determine linear slopes when peptide is >10% phosphorylated, so as a surrogate for activity, K_{d} (t₅₀P, the time at which 50% of the peptide is phosphorylated) for each sample dataset was calculated using the saturation binding model in GraphPad Prism (background constraint=no lysate control average fluorescence value; Bmax constraint=fluorescence value reached at plateau in the kinetic assay across all samples). Patient lysates were individually preincubated with 20 μM CX4945 for 30 min at room temperature before initiating kinase kinetic assays.

Vero-E6 infectivity assays. Vero-E6 cells were plated at 2.5K cells/well ˜18 h before treatment in 100 uL media on 96-well black plates with clear bottoms (NUNC 165305). Compounds were added to cells at 1.05× in DMEM+2% FBS media and incubated for 1-2 h. In a BSL3 facility (University of California-San Diego), SARS-CoV-2 virus was added at MOI of 0.025 and plates were incubated for 48 h. Cells were fixed and stained with anti-nucleocapsid (NP) antibody and Alexa-Fluor 594 secondary antibody with SytoxGreen (TFS) counterstain and imaged (Nikon). Percent infected cells (relative to DMSO vehicle) were determined by counting NP+ cells divided by SytoxGreen+ cells. In parallel, uninfected plates were assessed for viability after 48 h (CellTiter AQOne).

Compound structures are evaluated using molecular docking studies and in vitro radiometric kinase assays.

Anti-inflammatory activity is assessed in a THP-1 monocyte NF-KB reporter assay or in a cytokine secretion assay (e.g. IL-6, IL-8) in human PBMCs. Anti-inflammatory activity in human astrocytes is evaluated using a quantitative cytokine secretion assay (e.g. IL-6, IL-8).

Compound activity is assessed in vitro in AD patient-derived neurons, astrocytes, and/or microglia, and in vivo in APP/PS1 mice. In cells, the effects of CK2 inhibition are assessed on (a) activation of AD glia and (b) mitochondrial function in AD neurons (mitophagy, morphology). Amelioration of neuroinflammatory and dysfunctional mitochondrial phenotypes in AD preclinical models is evaluated. In mice, efficacy is assessed after 4-week drug treatment at 6, 9, and 12 months by measuring disease burden through behavioral measures (cognitive tests), biochemistry (cytokine levels, Ck2 substrate phosphorylation), and histology (Iba1/Gfap staining, neuronal loss).

### EXAMPLE

### CK2 as an Anti-Inflammatory Target and Activity of Natural Flavones

To demonstrate that CK2 is required for inflammation induction in astrocytes, CK2 enzyme activity was perturbed via knockdown, chemical inhibition, or overexpression of kinase-dead constructs as discussed in detail below. Knockdown of CSNK2A1/CK2α1 or CSNK2A2/CK2α2 via siRNA reduced the ability of IL1β to induce inflammation in primary astrocytes. In addition, two structurally unrelated CK2 inhibitors blocked inflammation in astrocytes treated with IL1β. Finally, overexpression of a CK2α1 or CK2α2 single point mutant that abrogates kinase activity also blocked inflammation. RNA-seq studies in acutely inflamed primary and control iPSC-derived astrocytes showed that inflammation signatures were significantly downregulated by treatment with a CK2 inhibitor. In contrast, CK2 inhibition did not have a large effect on gene expression in unstimulated (non IL1β-treated) astrocytes and microglia. These data revealed that CK2 plays a key role in the neuroinflammatory cascade.

To test the disease relevance of CK2 as a target for neurodegenerative disease, CK2 levels and activity were measured in AD and PD as discussed in more detail below. Elevated CK2 activity was observed in substantia nigra tissue from PD patients. Data showed CK2α2 levels trending higher in AD patient-derived astrocytes. These data indicated a correlation of CK2 overactivity with neurodegenerative disease.

CK2 as an anti-inflammatory target is further validated by showing that overexpression of kinase-dead CK2a/CK2A1 and CK2a′/CK2A2 reduces inflammation, and apigenin transcriptionally downregulates the NF-KB/IkB pathway.

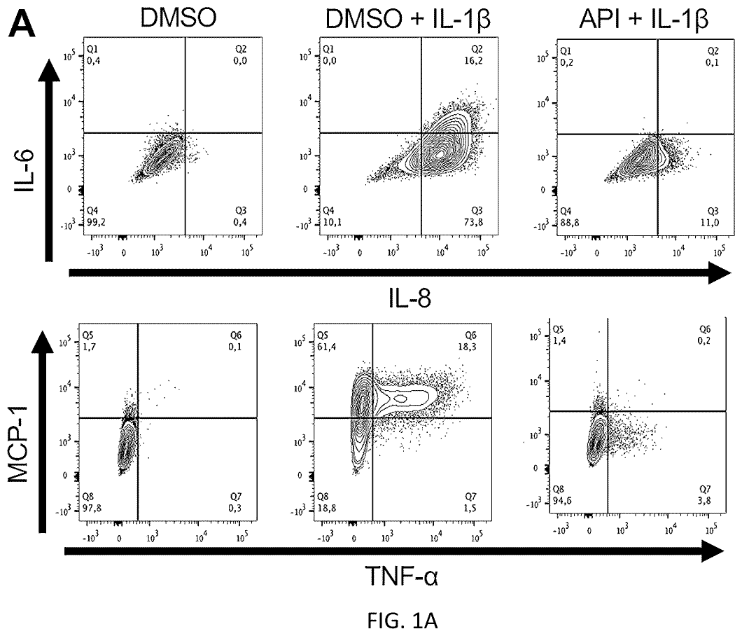

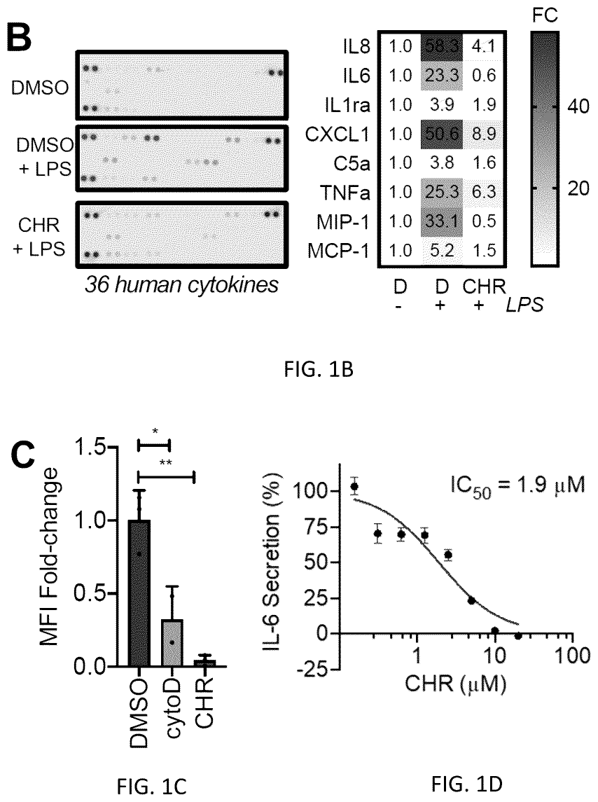

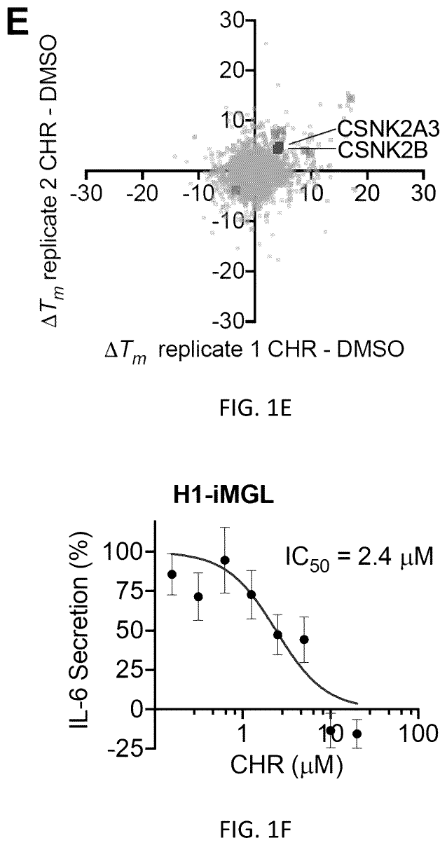

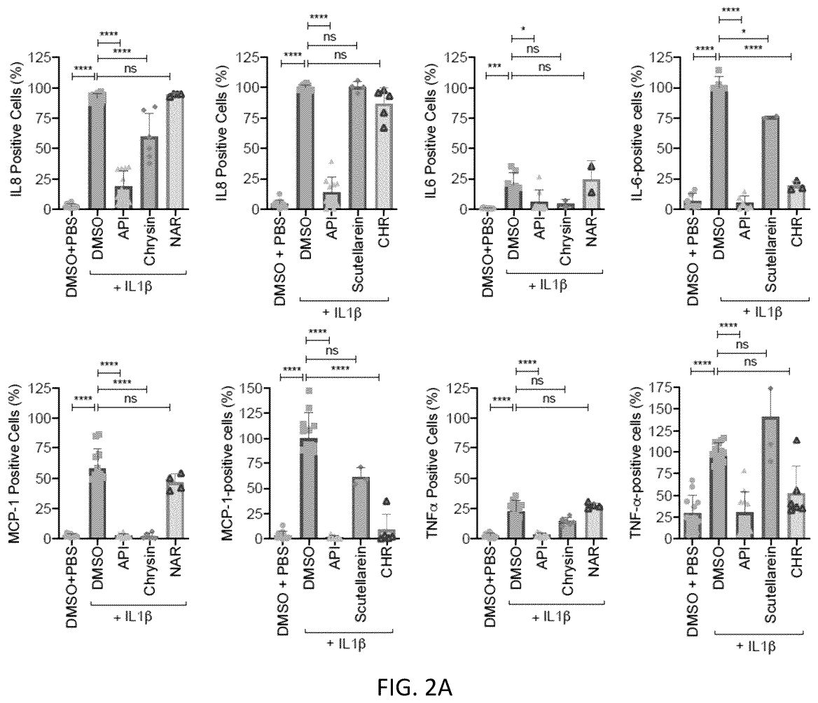

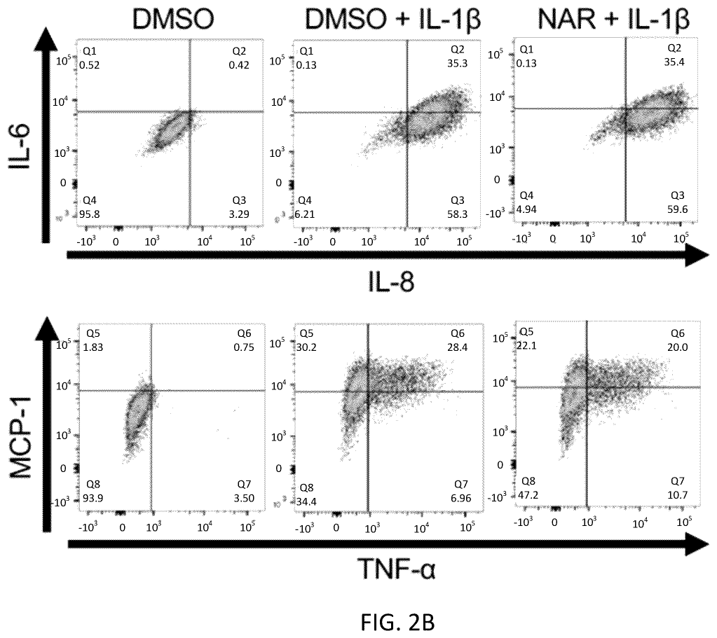

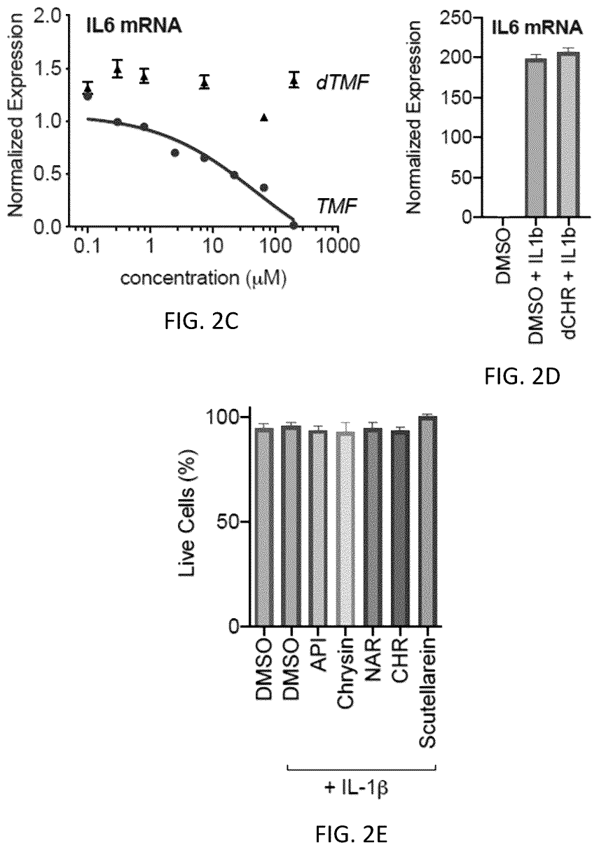

Primary human cerebellar astrocytes (HCA) were co-treated with 20 μM of each compound or vehicle (DMSO), 10 ng/mL IL1-β, and a protein transport inhibitor for 6 hours, and then processed for intracellular staining of pro-inflammatory cytokines IL-6, IL-8, TNF-α, and MCP-1. Results are shown in FIGS. 1-2 and Tables 2-3. Certain flavones like apigenin and trimethyl-apigenin (TMF) were active in reducing inflammation of primary astrocytes and iPSC-derived astrocytes (Table 2; in vitro IC₅₀ values are for CK2 as reported in the literature). In contrast, structurally similar flavanones like naringenin, homoeriodictyol and trimethoxyflavanone were inactive (Table 3).

TABLE 2
Most Active Flavones 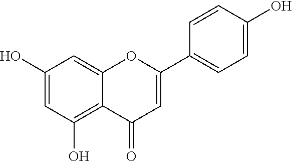 [SMILES: O=c1cc(-c2ccc(O)cc2)oc2cc(O)cc(O)c12]
apigenin CK2 IC₅₀ = 800 nM 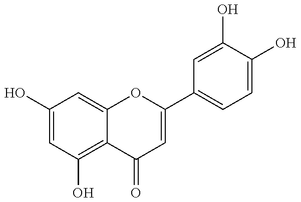 [SMILES: O=c1cc(-c2ccc(O)c(O)c2)oc2cc(O)cc(O)c12]
luteolin CK2 IC₅₀ = 500 nM 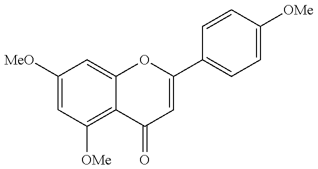 [SMILES: COc1ccc(-c2cc(=O)c3c(OC)cc(OC)cc3o2)cc1]
timethoxyflavone (TMF) 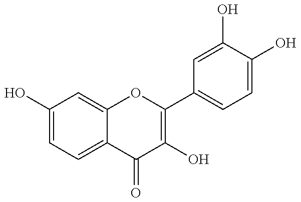 [SMILES: O=c1c(O)c(-c2ccc(O)c(O)c2)oc2cc(O)ccc12]
fisetin CK2 IC₅₀ = 350 nM 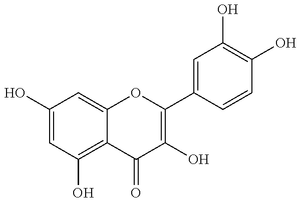 [SMILES: O=c1c(O)c(-c2ccc(O)c(O)c2)oc2cc(O)cc(O)c12]
quercetin CK2 IC₅₀ = 550 nM 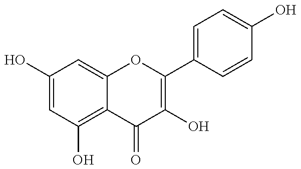 [SMILES: O=c1c(O)c(-c2ccc(O)cc2)oc2cc(O)cc(O)c12]
kaempferol CK2 IC₅₀ = 400 nM

TABLE 3
Inactive Flavones 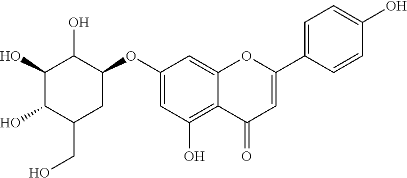 [SMILES: O=c1cc(-c2ccc(O)cc2)oc2cc(O[C@H]3CC(CO)[C@H](O)[C@@H](O)C3O)cc(O)c12]
glycosylated apigenin CK2 IC₅₀ > 40 μM

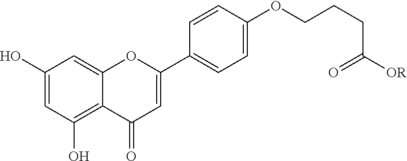

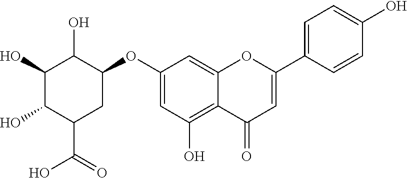 [SMILES: O=C(O)C1C[C@H](Oc2cc(O)c3c(=O)cc(-c4ccc(O)cc4)oc3c2)C(O)[C@H](O)[C@H]1O]

glucoronidatedd apigenin CK2 IC₅₀ = 16.6 μM 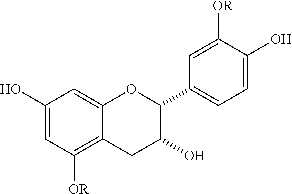
epicatechin 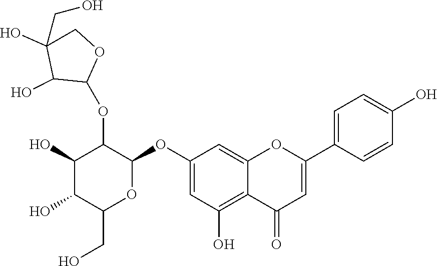 [SMILES: O=c1cc(-c2ccc(O)cc2)oc2cc(O[C@H]3OC(CO)[C@H](O)[C@@H](O)C3OC3OCC(O)(CO)C3O)cc(O)c12]
glycosylated apigenin 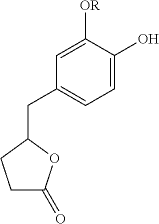
epicatechin metabolite 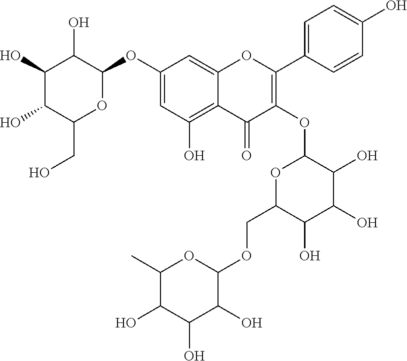 [SMILES: CC1OC(OCC2OC(Oc3c(-c4ccc(O)cc4)oc4cc(O[C@H]5OC(CO)[C@H](O)[C@@H](O)C5O)cc(O)c4c3=O)C(O)C(O)C2O)C(O)C(O)C1O]
glycosylated kaempferol 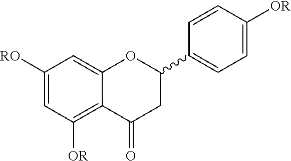
R = H, naringenin CK2 IC₅₀ > 40 μM R = Me, trimethoxyflavanone (dTMF)

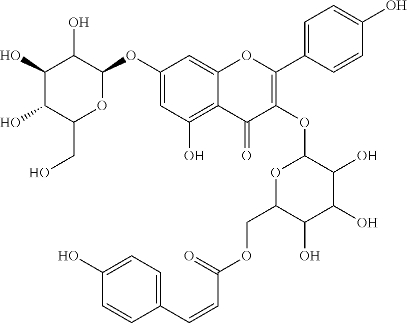 [SMILES: O=C(/C=C\c1ccc(O)cc1)OCC1OC(Oc2c(-c3ccc(O)cc3)oc3cc(O[C@H]4OC(CO)[C@H](O)[C@@H](O)C4O)cc(O)c3c2=O)C(O)C(O)C1O]

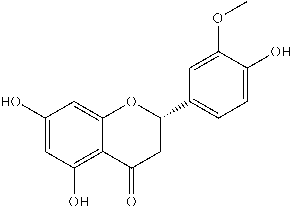 [SMILES: COc1cc([C@@H]2CC(=O)c3c(O)cc(O)cc3O2)ccc1O]

homoeriodictyol (dCHR) 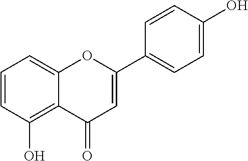 [SMILES: O=c1cc(-c2ccc(O)cc2)oc2cccc(O)c12]
7,4′-dihydroxyflavone

The presence of a C2-C3 double bond was the predominant determining factor for anti-inflammatory activity. For example, apigenin (API) strongly blocked IL1-β-induced IL-6, IL-8, TNF-α, and MCP-1 cytokine upregulation, unlike its flavanone analogue naringenin (NAR, FIG. 1A and FIG. 2B). Similar divergence in activity was observed with trimethylated apigenin (4′,5,7-trimethoxyflavone, TMF) and its corresponding inactive flavanone analogue 4′,5,7-trimethoxyflavanone (dTMF, FIG. 2C) and chrysoeriol (CHR) versus homoeriodictyol (dCHR, FIGS. 2A, 2D). For subsequent phenotypic testing, active flavones API, TMF, or CHR were used. The effects of CHR treatment on cytokine release in LPS-stimulated human induced pluripotent stem cell (iPSC) derived microglia (Abud et al., Neuron 2017, 94:278-293.e9) were assessed using a panel of 36 cytokines (FIG. 1B). CHR significantly blocked the release of multiple LPS-induced pro-inflammatory cytokines, including interleukins (IL-6, IL-8, IL1ra), chemokines (CXCL1, MCP-1, MIP-1), complement (C5a), and TNF-α (p<0.05, unpaired two-tailed t-tests). Altered phagocytic function of microglia is associated with neurodegenerative disease. CHR blocked microglial phagocytosis of pHrodo E. coli beads (P=0.0015, FIG. 1C, compared to phagocytosis inhibitor cytochalasin D). The anti-inflammatory effects of CHR (FIG. 1D) and TMF (FIG. 2C) were dose-dependent in astrocytes and microglia. There was no cytotoxicity associated with flavone treatment (FIG. 2E).

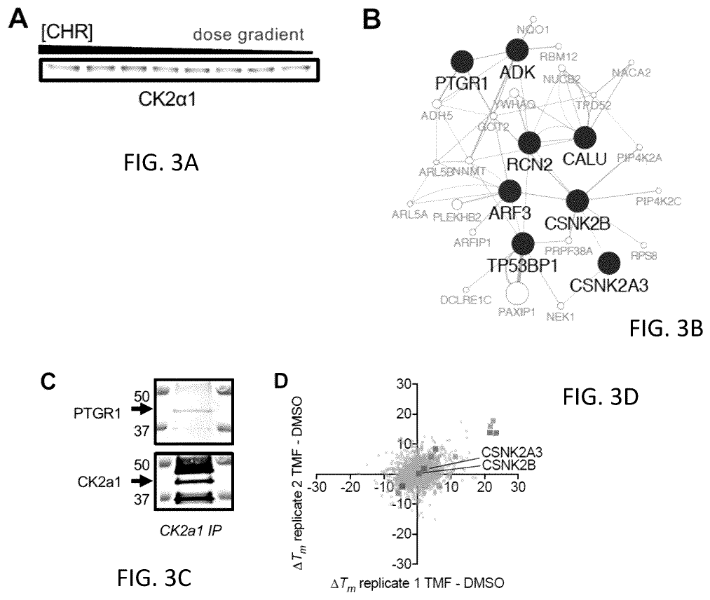

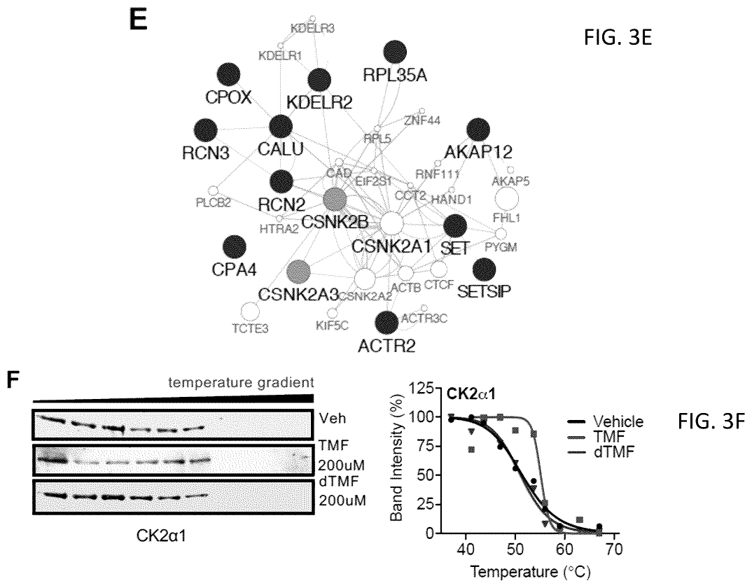

An unbiased proteomics approach was used to identify cellular targets of flavones in inflamed astrocytes. Thermal shift proteome profiling (TPP) identifies drug target engagement in cells by measuring ligand stabilization (or destabilization) of bound proteins against heat denaturation. In this assay, stabilized proteins can be either direct or indirect targets, while destabilized proteins are indirect targets. Lysates were extracted from IL1-β-treated human iPSC-derived astrocytes and treated with CHR or DMSO for 30 minutes. TPP was performed using LC-MS/MS with TMT10 labeling. Eight proteins were identified with a significant thermal shift (ΔTₘ) reproducible in two independent experiments (FIG. 1E). Two of the hits, CSNK2A3 (CK2α1P) and CSNK2B (CK2β), constitute components of the CK2 holoenzyme. CK2 is catalytically active either as a heterotetrameric complex composed of two alpha and two beta subunits or an alpha monomer. CSNK2A3 is nearly identical (99% cDNA homology) to CSNK2A1, while CSNK2A2 shares 77% identity with CSNK2A1. CSNK2A1 peptides were not detected by mass spectrometry in the TPP experiment. CK2α1 was confirmed to be dose-dependently stabilized by CHR in an isothermal dose response thermal shift assay (FIG. 1F, FIG. 3A) followed by Western blotting.

Network analysis of the other identified TPP hits (ADK, PTGR1, TP53BP1, CALU, RCN2, ARF3) showed high connectivity with each other and CK2 (FIG. 3B). ARF3 showed thermal destabilization indicative of an indirect hit. ADK and TP53BP1 physically interact with CK2, CALU is a known CK2 substrate, and RCN2 is a likely CK2 substrate. Co-immunoprecipitation was performed with CK2α1 and showed that it associates with PTGR1 (FIG. 3C).

The TPP experiment was repeated with another flavone compound, TMF. Eleven proteins were found with a significant thermal shift reproducible in two independent experiments (FIG. 3D). CSNK2A3 and CSNK2B were stabilized by TMF, but not enough to pass the stringent statistical significance requirements. Network analysis of the 11 hits and all 4 CK2 subunits, however, shows high connectivity (FIG. 3E). Among the 8 stabilized proteins, 6 are known CK2 substrates (RCN1, RCN3, SET and its paralogue SETSIP, CALU, AKAP12) and ACTR2 is a known interactor. Validation showed that CK2α1 is indeed a target of TMF (but not inactive flavanone dTMF) in the thermal shift assay followed by Western blotting (FIG. 3F).

These data demonstrated CK2 as the relevant target for further validation.

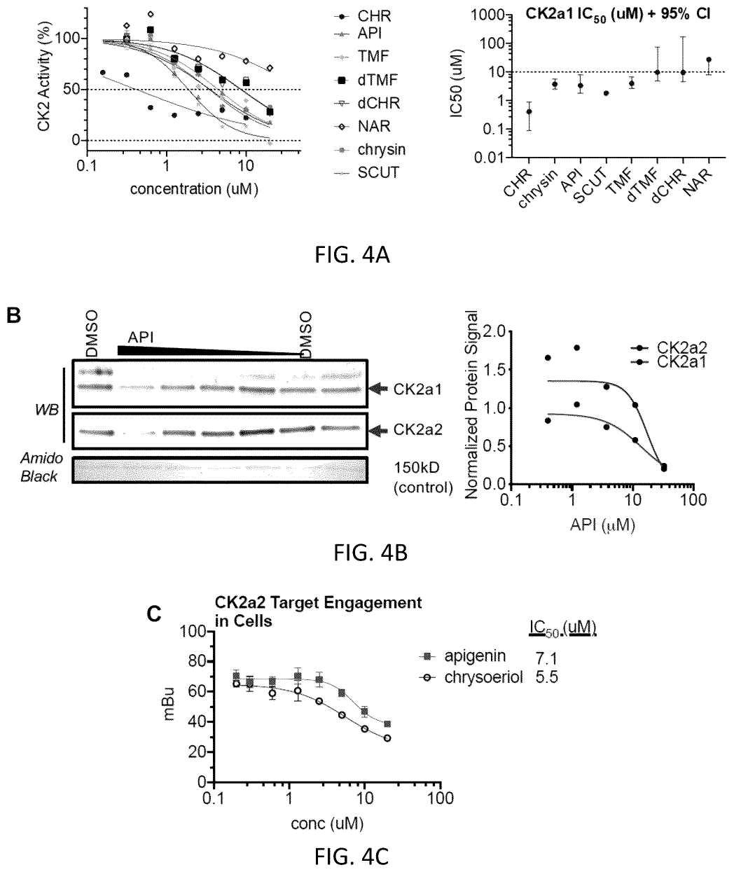

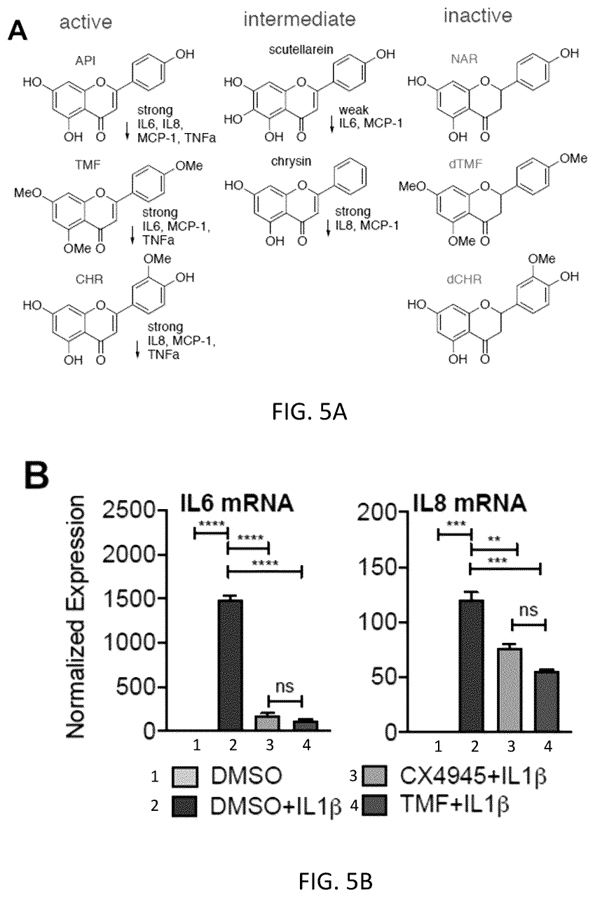

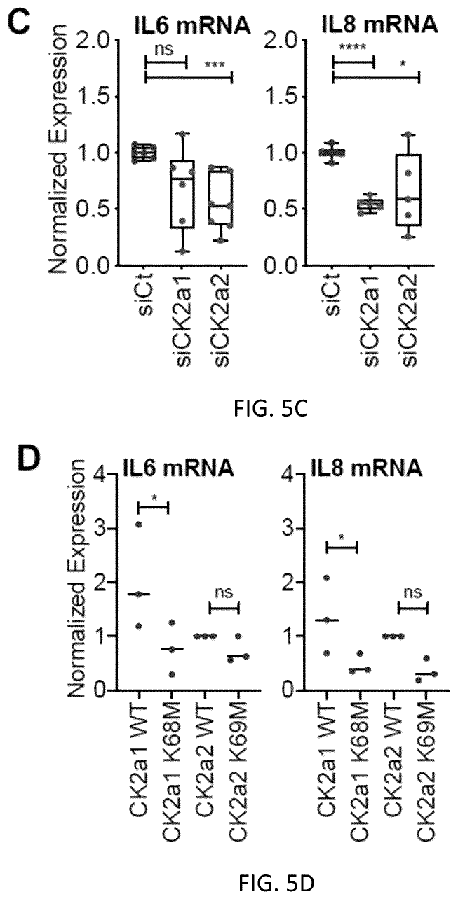

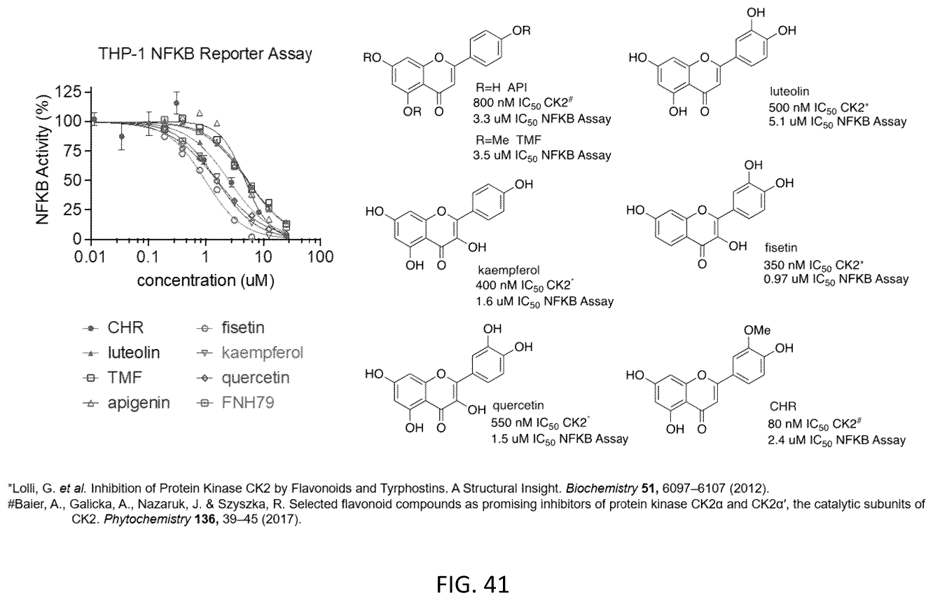

Flavones were verified to directly inhibit CK2 in cells via additional orthogonal methods. In astrocytes, API displaced a desthiobiotin-ATP probe from both CK2α1 and CK2α2 (FIG. 4B). CK2α2 target engagement of API (IC₅₀=7.1 μM) and CHR (IC₅₀=5.5 μM) in cells via NanoBRET (Vasta et al., Cell Chem Biol 2018, 25:206-214.el 1) was confirmed (FIG. 4C). Revisiting our structure-activity studies in the context of CK2 inhibition, we found that anti-inflammatory activity in astrocytes correlated well with CK2 inhibitory activity (FIG. 5A, FIG. 4A, FIG. 2B, FIG. 41). API, TMF, and CHR possessed IC₅₀ values ranging from 410 nM-4 μM (FIG. 4A) as well as strong suppressive activity against at least 3 out of 4 cytokines measured (FIG. 2B,D). Scutellarein and chrysin possessed intermediate IC₅₀ values ranging from 1.8-3.8 μM and intermediate activity, with variable suppression of 2 cytokines. Flavanones missing the key C2-C3 double bond identified earlier were completely inactive both as CK2 inhibitors and anti-inflammatory agents (NAR, dTMF, dCHR, IC₅₀>10 μM). Astrocytes were treated with TMF or CX4945, a potent synthetic CK2 inhibitor currently in clinical trials for cancer. It was found that both successfully blocked expression of IL6 and IL8 (FIG. 5B, one-way ANOVA with Tukey's post-hoc test).

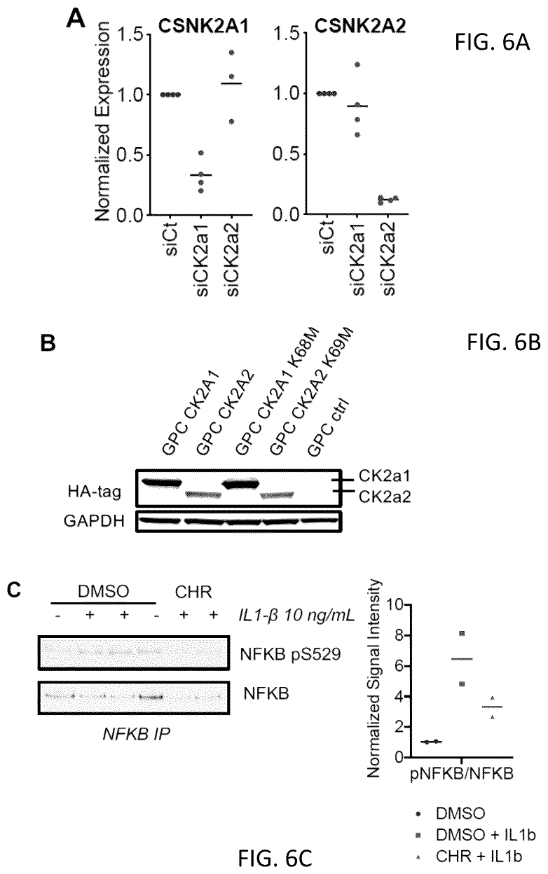

An evaluation of siRNA knockdown of individual CK2 paralogues CK2α1 or CK2α2 in human primary astrocytes was performed. CK2α/CK2α1 and CK2α′/CK2α2 are independent and distinct catalytic subunits of the CK2 holoenzyme that form a tetramer of 2 CK2A subunits and 2 regulatory subunits CK2B. They have different patterns of expression in the brain but unknown sub-functions. After 3 days, cells were treated with IL-1β for 5 hours and RNA was extracted to assess cytokine levels. Knockdown of either CK2α1 or CK2α2 reduced the ability of IL-1β to induce upregulation of pro-inflammatory cytokines IL6 and IL8 (FIG. 5C, FIG. 6A). Correspondingly, overexpression of a “kinase-dead” form of CK2 could affect inflammatory induction in astrocytes was considered. Glial progenitor cells were transduced with an LV-CK2α1 or CK2α2 wild-type (WT) or single point mutant (K68M and K69M, respectively), differentiated them for 4 weeks to produce mature astrocytes, then stimulated with IL1-β for 5 hours. CK2α1-K68M significantly abrogated inflammation relative to WT, and a similar but non-significant trend for CK2α2-K69M versus its WT (FIG. 5D, FIG. 6B). Three days after nucleoporation with siRNA, cells were pretreated with DMSO or TMF for 1 hour and then stimulated with IL1β for 5 hours. Inflammation was reduced by CK2α1 and CK2α2 knockdown. In addition, TMF's anti-inflammatory effect was partially rescued upon individual CK2α1 or CK2α2 knockdown. This shows that TMF reduces inflammation mainly through inhibition of CK2. CK2 as an anti-inflammatory target was validated by showing that overexpression of kinase-dead CK2a and CK2a′ reduces inflammation, and apigenin transcriptionally downregulates the NF-KB/IkB pathway.

These data demonstrate that CK2 is the relevant cellular target of anti-inflammatory flavones, and that CK2 is an important upstream pro-inflammatory regulator in astrocytes.

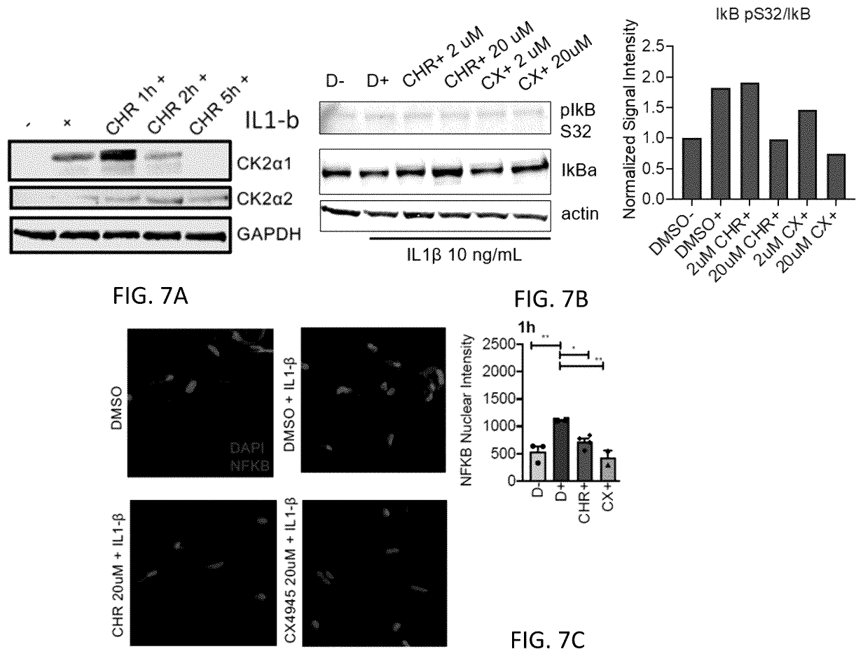

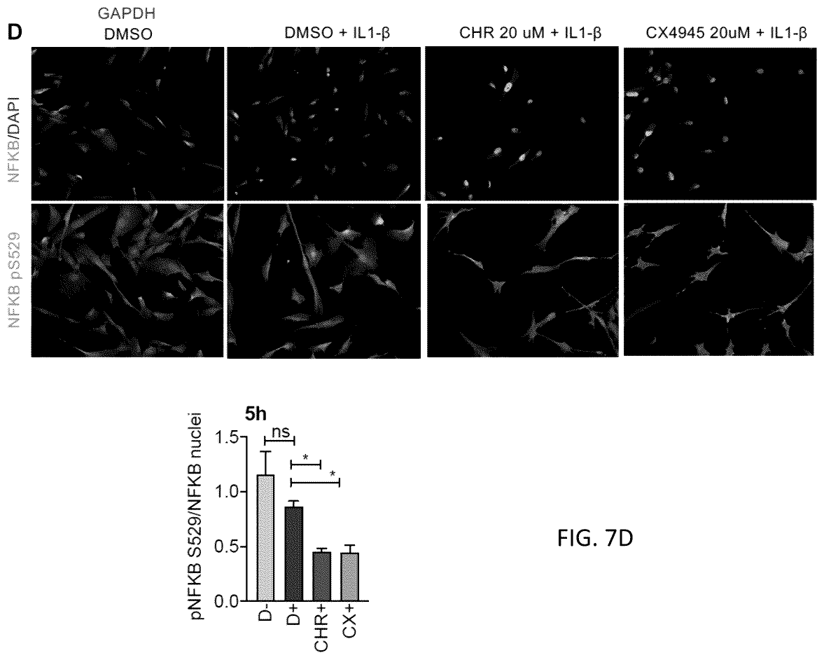

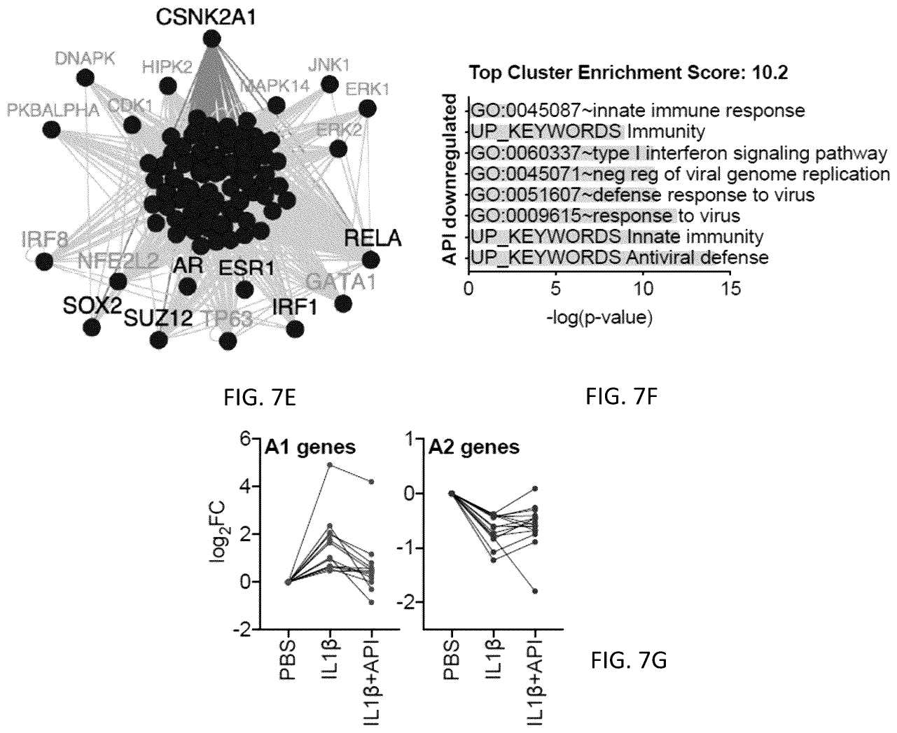

An increase in CK2 levels in human primary astrocytes stimulated with IL1-β was observed, with CK2α1 peaking at 1 h after induction and reduced to baseline with CHR treatment after 5 hours (FIG. 7A). This indicates that CK2 is an immune-responsive kinase in astrocytes.

Several proteins involved in the innate immune response have been characterized as substrates of CK2, including IκB S32 and NF-κB S529. NF-κB is a master transcriptional regulator of the immune response, downstream of a broad repertoire of exogenous stimuli, including Toll-like receptors, IL-1R, and TNFR. IκBα is a negative feedback regulator and acts as an inhibitor of the NF-κB program by sequestering NF-κB in the cytoplasm. IκBa S32 phosphorylation is necessary for degradation of IκBα by pro-inflammatory stimuli. IκB S32 levels were increased with inflammation and decreased with CK2 inhibitors CHR and CX4945 after 5 hours (FIG. 7B). This is consistent with CK2 acting downstream of IL1-β to phosphorylate IκB S32, with CK2 inhibition leading to a reduction of pIκB and subsequent stabilization of total IκBα.

To investigate the potential effects of CK2 activity on NF-κB, NF-κB and NF-κB pS529 were measured by immunofluorescence in activated astrocytes. NF-κB nuclear intensity increased after 1 h of IL1-13 treatment, but this was blocked by CHR or CX4945 treatment (FIG. 7C). At this timepoint, NF-κB pS529 levels were similar across all treatments, possibly as a result of slower turnover by pNF-κB species. After 5 hours, both CHR and CX4945 reduced pNF-κB nuclear intensity relative to total NF-κB (FIG. 7D). At the intermediate timepoint of 2 h post-induction with IL1-β, we detected an increase in pNF-κB relative to NF-κB via immunoprecipitation (IP) followed by immunoblotting; this signal was suppressed by CHR co-treatment (FIG. 6C). Overall, these results reflect the dynamic regulation of NF-κB by CK2 and indicate that CK2 might play an important role in sustaining NF-κB-driven immune response.

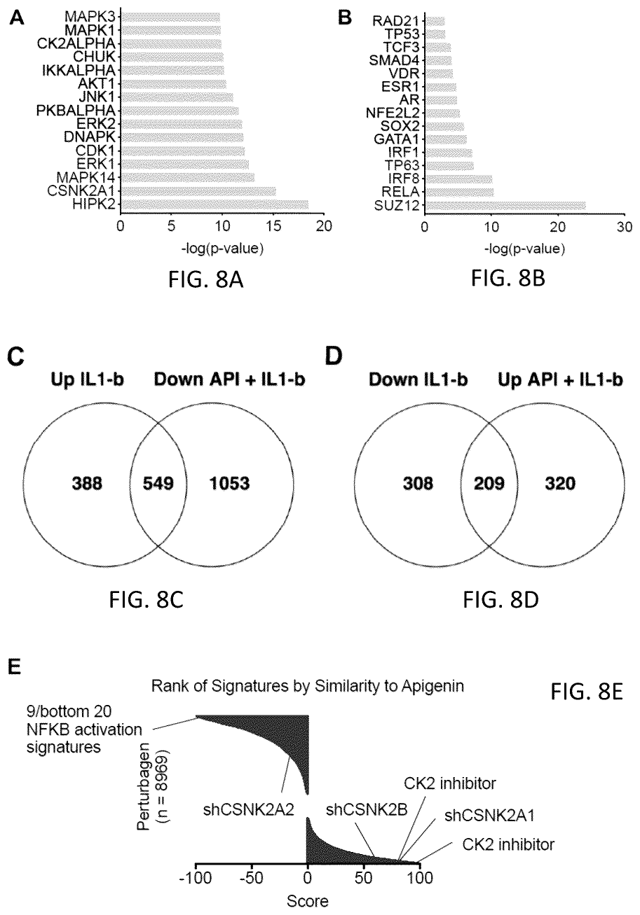

To further investigate the mechanistic role of CK2 in driving inflammation in glia, RNA sequencing (RNA-seq) was performed in human primary astrocytes stimulated with IL1-β and API or vehicle for 5 h. Differential expression (DE) analysis showed 533 genes downregulated and 938 genes upregulated upon activation (P<0.05, |log 2FC|>1). This inflammatory signature was strongly enriched in genes regulated by CK2, which appeared twice in the top 15 hits by Kinase Enrichment Analysis (P=5.8×10⁻¹⁶ and P=1.19×10⁻¹⁰, FIG. 8A). Indeed, network analysis shows that CK2 plays a central role in regulating many of the downstream-enriched transcription factor (TF) modules, including RELA/NF-κB, IRF1, and polycomb repressive complex subunit SUZ12 (FIGS. 7E, 8B). IRF1 is another key TF of innate immunity and SUZ12/EZH2 have been shown to regulate NF-κB target gene expression.

Next, gene expression of cells treated with API+IL1-β versus IL1-β alone was compared, and it was found that 531 genes were upregulated and 1603 genes were downregulated (P<0.05, |log 2FC|>1). Notably, API reversed a significant fraction of the gene expression changes induced in activated astrocytes (58% of the genes upregulated by IL1-β, P=10⁻¹⁰³⁷ FIG. 8C; and 40% of the genes downregulated by IL1-β, P=2.7×10⁻²²⁶, FIG. 8D). Among all of the API-downregulated genes, the top cluster of enriched terms by functional gene annotation analysis was related to the innate immune response (FIG. 7F). Querying DE genes following API treatment in the CMap database, we found that multiple CK2 inhibitor and CK2 knockdown signatures exhibited high similarity scores (FIG. 8E). In contrast, nine out of the top 20 most dissimilar signatures in the CMap database related to NF-κB activation. These results indicate the CK2 inhibition effectively attenuates the inflammatory response in astrocytes.

CK2 inhibition modulation of the A1/A2 polarization of reactive astrocytes was evaluated. A1 astrocytes are neurotoxic, forming as a result of chronic inflammation, acute CNS injury, and neurodegenerative diseases, while A2 astrocytes are neuroprotective, promoting tissue repair following cerebral ischemia. Of the 14 A1 genes significantly upregulated by IL1-β in primary astrocytes, 10 of them were significantly downregulated by API (P<0.05, two-tailed t-tests, FIG. 7G). Similarly, of the 13 A2 genes downregulated by IL1-β, 12 were upregulated by API (P<0.05, two-tailed t-tests, FIG. 7G). This shows that CK2 inhibitors suppress the gene expression program underlying the neurotoxic astrocyte phenotype and promote a neuroprotective astrocyte-like transcriptional profile.

Taken together, these data demonstrate that CK2 is a potent master regulator of inflammation in glia upstream of NF-κB. At the transcriptional level, CK2 maintains the polarization of reactive astrocytes towards pro-inflammatory, neurotoxic pathways at the expense of a neuroprotective response. Thus, CK2 inhibition effectively reverses the A1/A2 polarization of inflamed astrocytes by restraining tissue-damaging and promoting tissue-repair transcriptional programs.

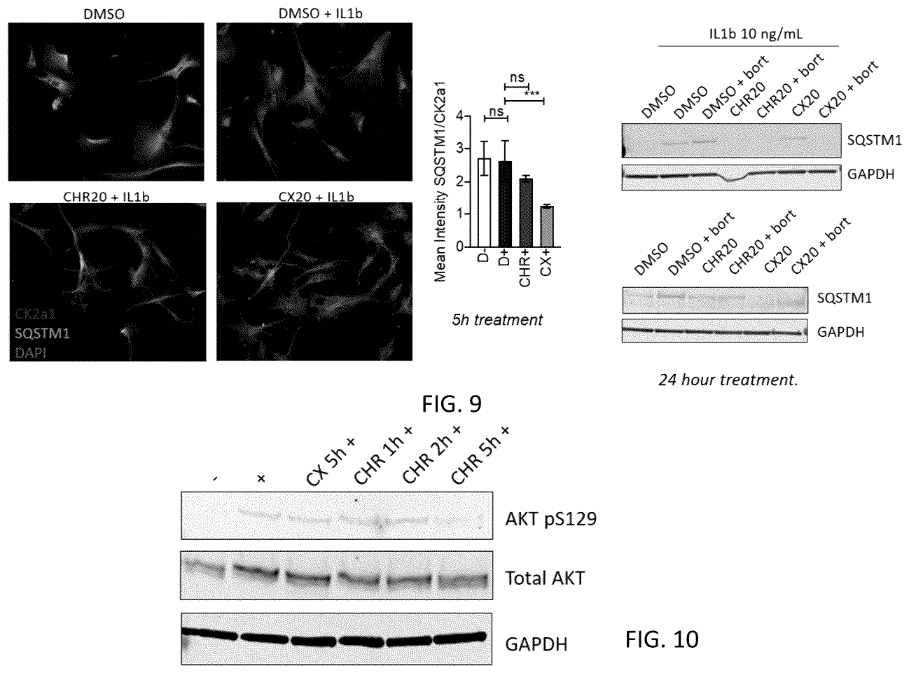

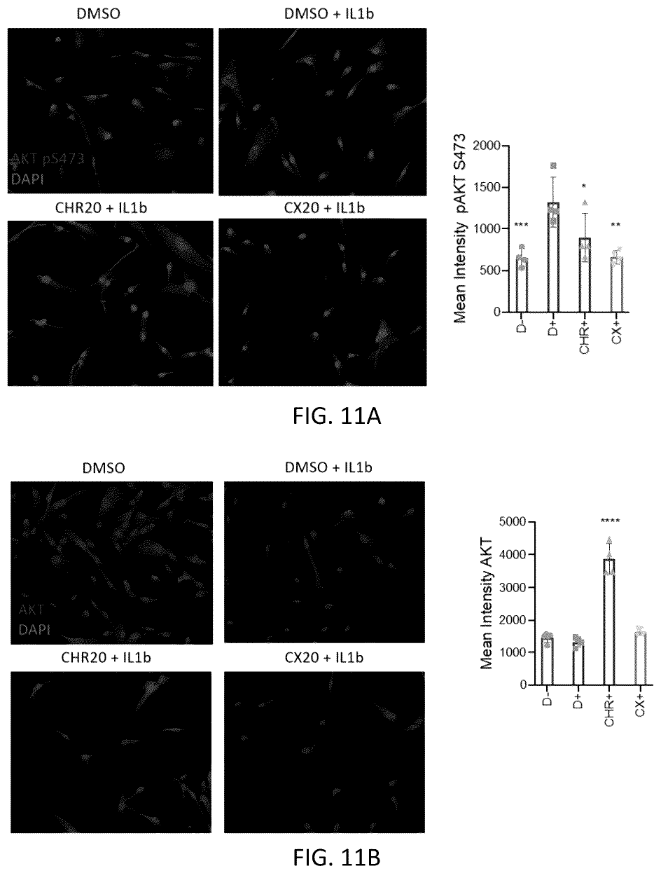

We discovered other CK2-dependent biomarkers in cells, including some induced by inflammation. SQSTM1/p62 was induced by IL1-β and reduced by CK2 inhibitor treatment, as determined by immunofluorescence in primary astrocytes (FIG. 9, left) and western blotting (FIG. 9, right). This process was not proteasome-dependent as bortezomib treatment did not rescue the effect.

CK2 inhibitor CHR reduced AKT pS129 levels that are induced in primary astrocytes stimulated with IL1-β for 5 h (FIG. 10). AKT pS129 is a known direct CK2 substrate.

CK2 inhibitors CX-4945 and CHR reduced AKT pS473 levels in primary astrocytes stimulated with IL1-β for 5 h as determined by immunofluorescence staining (mean±s.e.m., n=4) (FIG. 1A). AKT pS473 is a known indirect biomarker for CK2 activity.

Other potential CK2 substrates from phosphoproteomics and proteomics experiments with TMT-labeling in H1-derived astrocytes treated with DMSO, DMSO+IL1-β, or CHR 20 μM and IL1-β include (i) significantly up phosphoproteins in DMSO+1L1b group relative to DMSO and also down with CHR+IL1-β relative DMSO+IL1-β (1 hour)—CRIP2, DIDO1, SVIL; and (ii) significantly down phosphoproteins by CHR+IL1-β in both 1 h and 5 h timepoints relative to DMSO+IL1-β—PI4K2A, TBC1D15.

Having established that CK2 regulates neuroinflammation in human glia, the question of whether neurodegenerative diseases exhibit dysregulated CK2 activity was investigated. Astrogliosis and chronic inflammation underlie pathogenesis in both Parkinson's disease and Alzheimer's disease. Neuroinflammation also is found in Huntington's disease.

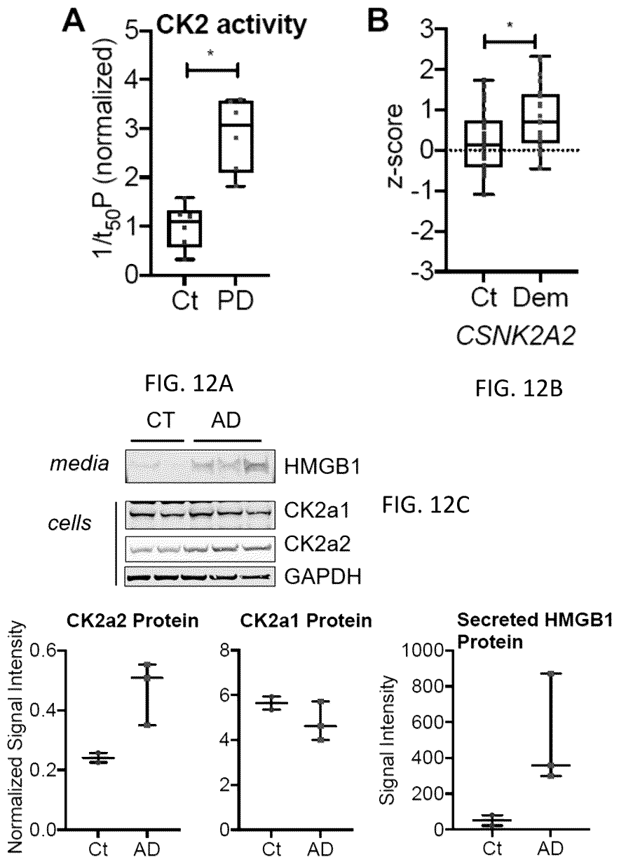

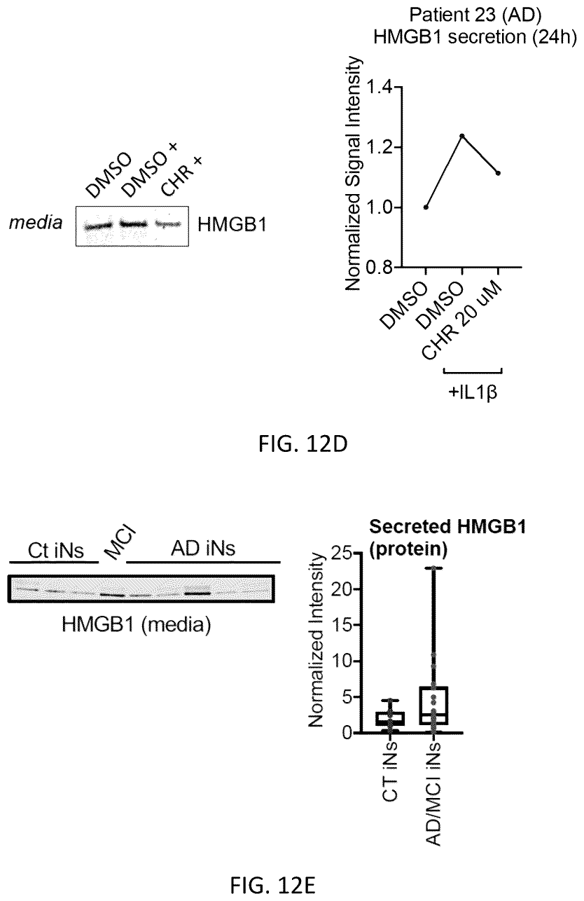

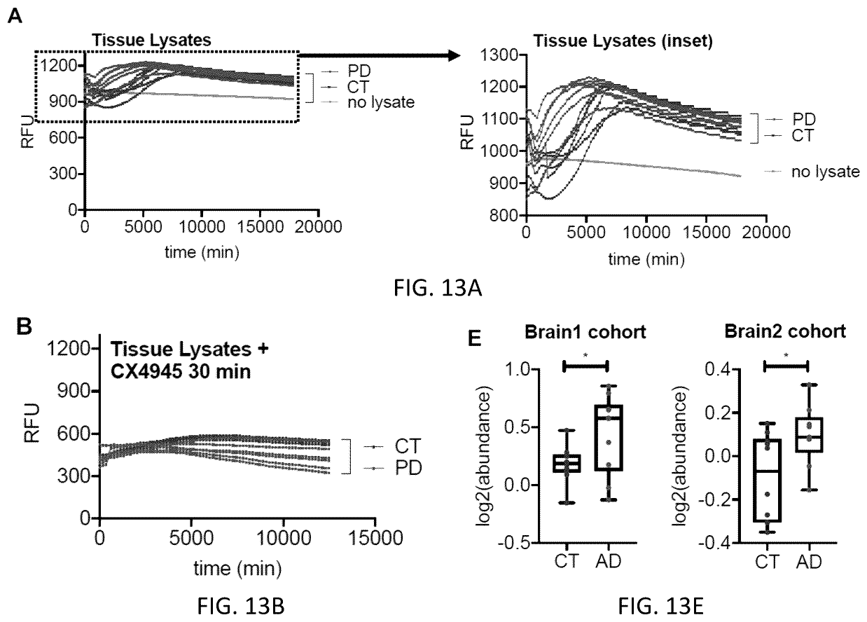

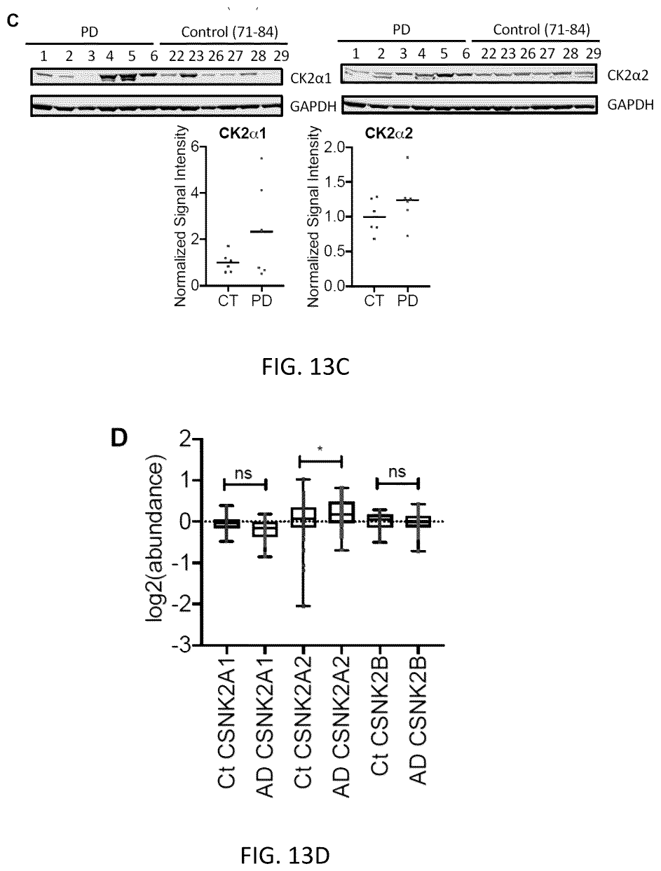

To demonstrate the disease relevance of CK2 as a target for neurodegenerative disease, CK2 levels and activity were measured in AD and PD (FIGS. 12A-12D). CK2 enzymatic activity was quantified in substantia nigra postmortem tissue from a set of PD patient samples (n=6) and age-matched controls (n=6), and a significant upregulation in PD was found (FIG. 12A, 13A, 13B). At the protein level, CK2α1 and CK2α2 were similar in expression in PD and controls, indicating that differential CK2 activity was observed as a result of post-translational regulatory mechanisms (FIG. 13C). Preliminary data showed CK2α2 levels trending higher in AD patient-derived astrocytes. These data indicated a correlation of CK2 overactivity with neurodegenerative disease.

Transcriptome profiling data from the Aging, Dementia, and TBI RNA-seq study showed that that CSNK2A2 was significantly upregulated in dementia patients compared to healthy controls (P=0.024, two-tailed t-test, FIG. 12B). Protein expression data from the UPP Proteomics Study confirmed that CK2α2 was also upregulated in AD patients compared to controls (P=0.015, one-way ANOVA with Sidak's post-hoc test, FIG. 13D). Interestingly, CK2α2 expression is enriched in the brain relative to other tissues, unlike CK2α1.

To further investigate this CK2 dysregulation in an in vitro AD model, iPSC-derived astrocytes were generated from a cohort of 6 AD patients and 5 age-matched controls. CK2α2 levels trended higher in AD astrocytes (FIG. 12C). HMGB1 is a chromatin-binding factor normally found in the nucleus, but it can also act as a pro-inflammatory damage-associated molecular pattern (DAMP) when it is secreted, a process that is induced via phosphorylation. While there has been evidence that CK2 phosphorylates HMGB1 homologues, it's unclear whether this occurs in humans. However, there was a question of whether CK2 could modulate HMGB1 phosphorylation and secretion via phosphatase PP2A. PP2A dephosphorylates HMGB1, while CK2 phosphorylates and activates protein SET, an inhibitor of PP2A. HMGB1 was detected in media from AD astrocytes in the absence of inflammatory stimuli (FIG. 12C). Furthermore, CHR treatment reduced HMGB1 secretion in AD astrocytes (FIG. 12D). This is consistent with prior data showing reduction of HMGB1 release by kaempferol, another flavone with CK2 inhibitory activity.

Although astrocytes and microglia are the principal immune-responsive cells of the brain, neurons are also capable of engaging innate immune pathways, including via HMGB1 secretion. Induced neurons (iNs) derived from AD patient and control fibroblasts were generated, and secretion of HMGB1 was observed trending higher from AD iNs than age-matched control iNs (FIG. 12F, two-tailed Welch's t-test, P=0.053). This is consistent with prior studies showing that HMGB1 is also released in aged rodent brains and CSF. Analysis of published protein expression data (Higginbotham et al., bioRxiy 2019, 14:806752) from two Emory ADRC cohorts confirmed that HMGB1 was significantly upregulated in AD patients compared to controls (P=0.037 and P=0.026, two-tailed t-tests, n=9-10 per group, FIG. 13E). Gene expression data in a cohort of AD iNs and age-matched controls shows that levels of CSNK2A1 and CSNK2A2 trend higher in AD neurons.

Taken together, these data demonstrate that CK2 activity is upregulated in neurodegenerative diseases like PD and AD. In AD patient-derived astrocytes and neurons, CK2 levels correlate with higher secretion of DAMPs such as HMGB1, which could be abrogated via small molecule CK2 inhibition.

Thermal shift assays (TSA) in cell extracts from iPSC-derived astrocytes treated with flavones showed that apigenin, TMF, and chrysoeriol (another natural flavone) bind CK2a, while naringenin and trimethyl-naringenin do not. Furthermore, the stabilization of CK2 by active natural flavones was dose-dependent.

CK2a levels were found to increase upon IL1β stimulation of astrocytes. The commercial CK2 inhibitor, CX4945, reduced IL1β-induced inflammation in astrocytes to a similar degree as TMF. CX4945 is structurally unrelated to apigenin, but also acts as an ATP-competitive inhibitor of CK2a (engaging with the ATP-binding pocket).

A kinase capture experiment showed that active flavones inhibit CK2a/a′ by binding in the ATP-binding pocket. Capture of CK2a1 and CK2a2 by a covalent desthiobiotin probe (ActivX) was dose-dependently blocked by API (FIG. 4B).

CHR treatment reduced the phosphorylation of biomarkers AKT pS129, and NF-KB pS529 (CK2 substrates), which showed target engagement and inhibition of CK2 in cells (FIGS. 6C, 7B, 7D, 14, 15).

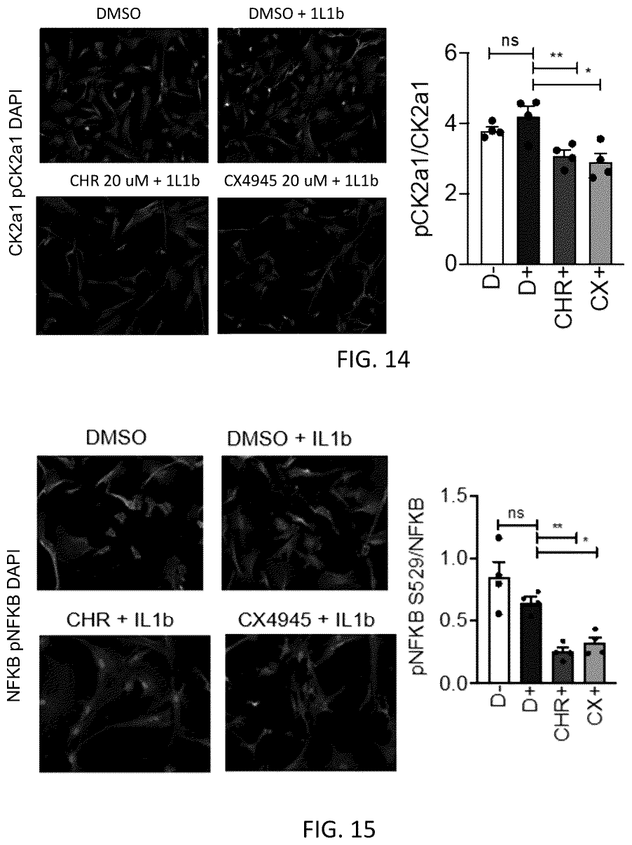

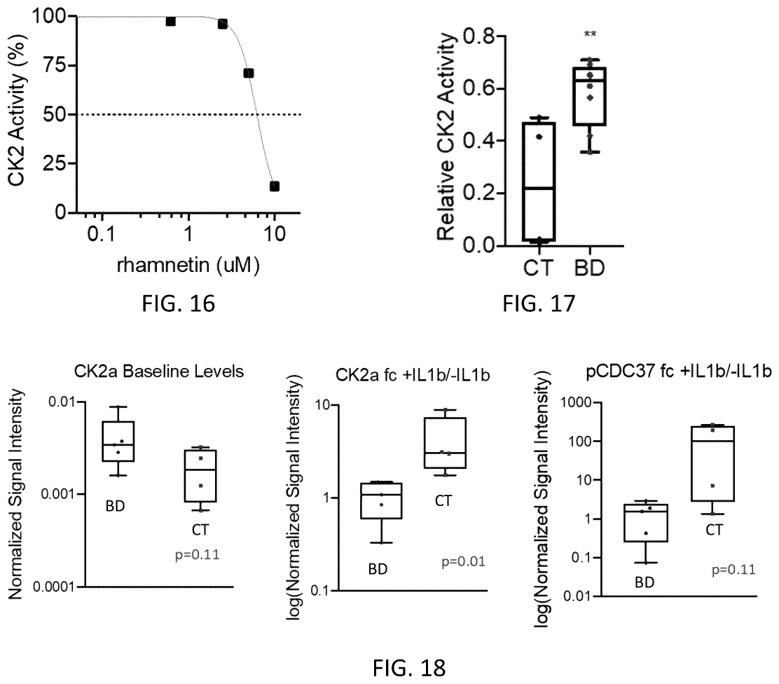

In other studies, pCK2/CK2 and pNF-κB/NF-κB were reduced by CHR (20 μM) and CX4945 (20 μM) treatment for 5 h in nuclei of primary human astrocytes (FIGS. 14, 15). Rhamnetin, a CK2 inhibitor (IC₅₀=6.2 μM) was shown to inhibit phosphorylation of a commercial synthetic substrate peptide (AssayQuant) (FIG. 16).

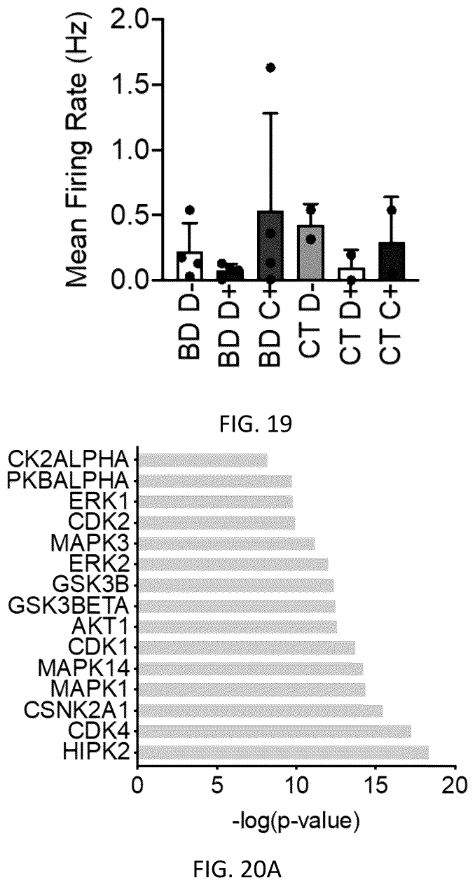

Bipolar disorder (BD) patient iPSC-derived astrocytes were shown to have higher CK2 levels and activity versus controls (CT) (FIGS. 17 and 18). CK2A1 baseline protein levels (normalized GAPDH trended higher in BD astrocytes compared to controls (FIG. 18). Additionally, CK2A1 levels of phosphorylation of CK2 substrate CDC37-513 were not increased by IL1-β activation in BD astrocytes, unlike in controls. CHR-pretreated astrocytes were shown to support higher neuronal activity (FIG. 19). The astrocytes were pretreated for 5 h with vehicle/vehicle, IL1β/vehicle, or IL1β/CHR 20 μM and evaluated 18 hours after plating the astrocytes on iGluta; two control iPSC-derived astrocytes (59, 61) and 4 bipolar iPSC-derived astrocytes (51, WT, TC, RS) were evaluated. The results show that the mean firing rate was reduced in rate in iGluta neurons co-cultured with BD or CT astrocytes pre-treated with IL1-β (D+ vs. D−). Conversely, neuronal activity is increased with CHR and IL1-β-pretreated (C+) BD astrocytes, compared to vehicle (D+). Activity of CT astrocytes is also renormalized (C+vs. D+).

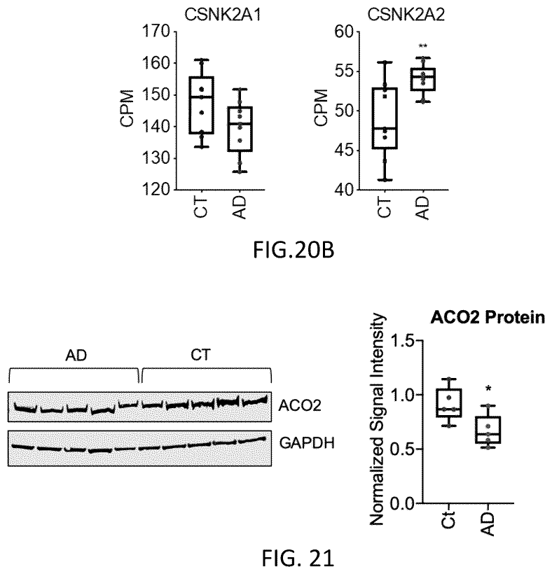

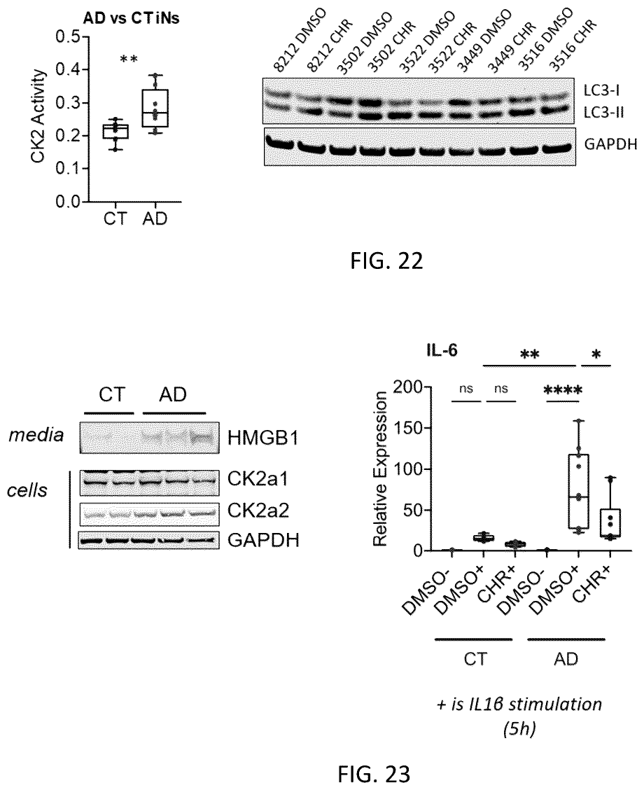

AD patient fibroblast-derived induced neurons (iNs) were shown to have higher CK2 levels and activity (kinase enrichment analysis of transcriptomics data) vs. CT (FIGS. 20A-20B). The AD patient fibroblast-derived iNs also exhibited higher secretion of damage-associated molecular pattern protein HMGB1 (n=2 CT, n=3 AD) (FIG. 12C), as well as secretion of HMGB1 in iNs from AD patients (n=5) and one patient that later developed mild cognitive impairment (MCI) compared to cognitively normal controls (n=3) (FIG. 12E). CK2 indirectly boosts the phosphorylation and subsequent secretion of HMGB1 through SET, inhibitor of phosphatase PP2A. The AD patient fibroblast-derived iNs exhibited mitochondrial defects as reflected by a reduced mitochondrial matrix protein level compared to age-matched controls (marker ACO2, FIG. 22).

AD patient-derived induced neurons showed increased CK2 activity via Cyclex CK2 ELISA assay in cellular lysates (FIG. 20). CK2 inhibition with CHR (24 h) increased LC3-II (marker of autophagy/mitophagy) in AD patient-derived neurons (FIG. 22). This shows that CK2 inhibition could improve mitochondrial dysfunction in AD neurons.

AD patient iPSC-derived astrocytes not only had a basal inflammatory state correlated to increased CK2 levels and HMGB1 secretion (FIG. 9E), but also showed hyperactivation in response to IL1-β stimulation (FIG. 23). AD astrocytes showed increased IL6 expression after IL1-β stimulation compared to controls, and IL6 expression was reduced by CHR treatment in both controls and AD astrocytes (one-way ANOVA, *P<0.05, **P<0.01, ****P<0.0001).

### Example 2

### Structure-Activity Relationships and Design of Novel Chemical Entities (NCEs)

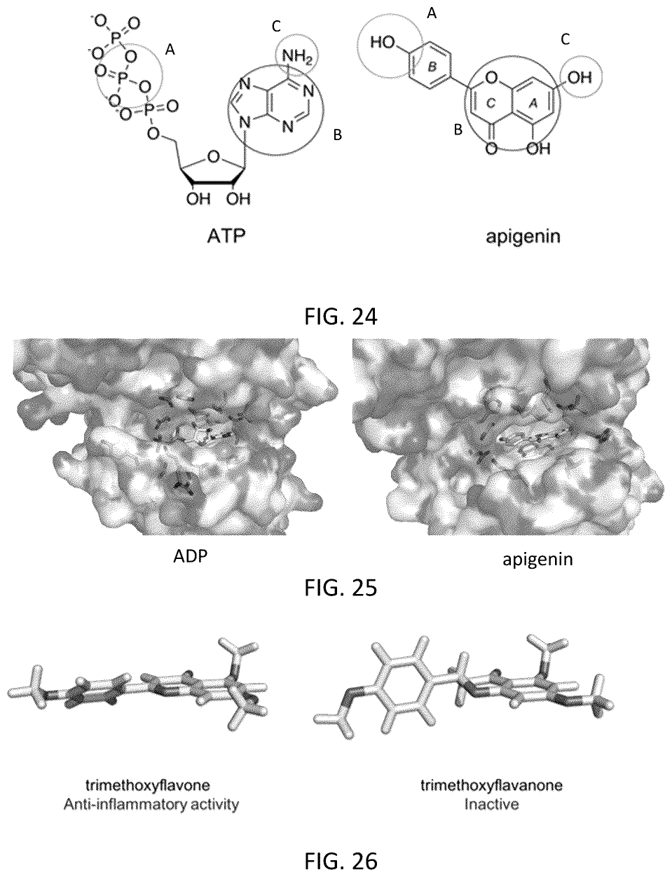

A published crystal structure of apigenin bound to human CK2a1 shows that anti-inflammatory activity correlates with relative binding affinity to CK2 (computational docking studies) as well as published IC₅₀ values. The chromene scaffold of flavones is integral for activity: the 4′-OH (—O⁻ at physiological pH) mimics the negatively charged phosphate of ATP, while the bicyclic chromene group acts like adenine of ATP and the other —OH groups, particularly at position 5, make further hydrogen bonding contacts with nearby residues (FIGS. 24, 25). This is likely because flavanones or epicatechins do not possess the right shape (flat) required to fit in the ATP-binding pocket of CK2a (FIG. 26). This observation is supported by the literature, which shows that naringenin has >50 fold higher IC₅₀ than apigenin. Moreover, glycosylated or glucoronidated derivatives and metabolites are inactive because: a) they are highly likely to be brain-impermeable (a claim also backed up by computational chemistry calculations) and b) their CK2 inhibitory activity in in vitro kinase assays is much lower (see Table 3). Another key feature is 4′-OH substitution; removing this group or methylating it reduces the anti-inflammatory activity and increases the CK2 IC₅₀ value (chrysin, acacetin and diosmetin in Table 4).

TABLE 4
Less Active Natural Flavones

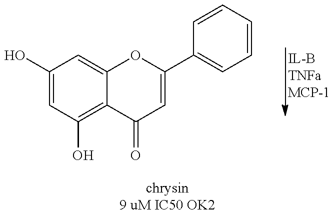 [SMILES: O=c1cc(-c2ccccc2)oc2cc(O)cc(O)c12]

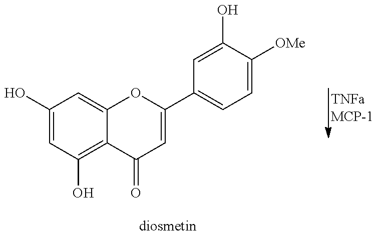 [SMILES: COc1ccc(-c2cc(=O)c3c(O)cc(O)cc3o2)cc1O]

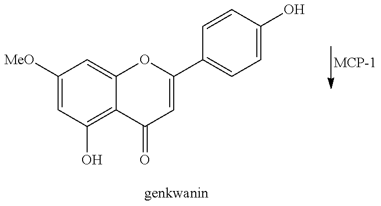 [SMILES: COc1cc(O)c2c(=O)cc(-c3ccc(O)cc3)oc2c1]

Two large in silico screens of 1.2 million Core Screening Compounds from Chembridge and 7 million Screening Compounds from Molport for docking into the ATP-binding pocket of CK2 (PDB: 3AMY and 3NGA) using Phase and Receptor-Based Virtual Screening (Schrodinger Maestro). The top 0.1% of compounds with the highest predicted binding affinity as well as favorable predicted physicochemical properties were filtered. NCEs were designed as analogs of the flavone scaffold to maximize solubility, bioavailability and blood brain barrier (BBB) permeability and they were pre-screened using crystal structure-based virtual docking studies using Schrodinger Maestro. Adding additional substituents on the A or C rings increases hydrophobic and other types of contacts with residues lining the ATP-binding pocket, increasing potency (Table 1 supra).

### Example 3

### Synthesis Schemes of Flavone Derivatives and Overview of Biological and Pharmacokinetic Property Evaluation

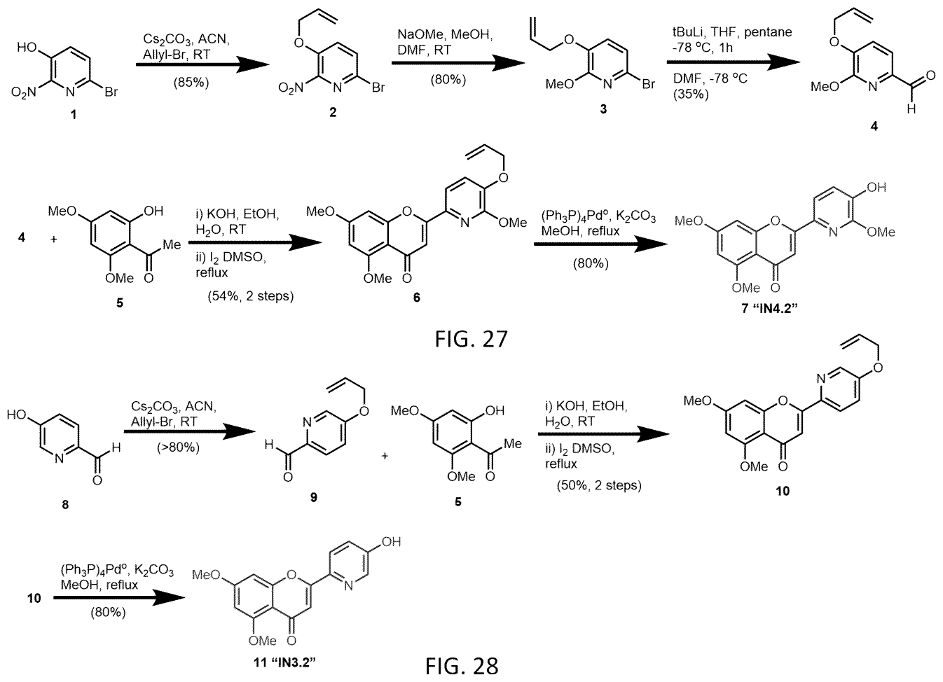

Exemplary synthetic schemes for several flavone derivatives (NCEs) shown in Table 1 are provided in FIGS. 27-34, respectively. In the synthetic schemes, solid reaction arrows indicate reactions that were performed, and dashed reaction arrows indicate prophetic reactions.

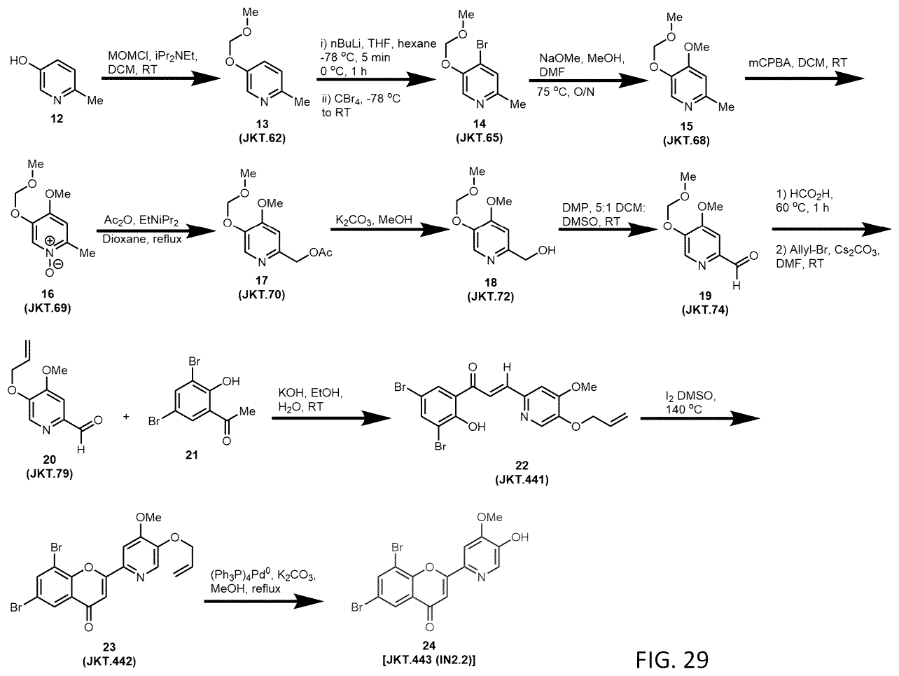

FIG. 27 shows a synthetic route for compound IN4.2 according to formula I where R² and R⁴ are —OR^{a}, and R⁶ is substituted pyridyl. FIG. 28 shows a synthetic route for compound IN3.2 according to formula I where R² and R⁴ are —OR^{a}, and R⁶ is substituted pyridyl. FIG. 29 shows a synthetic route for compound IN2.2 (JKT.443) according to formula I where R¹ and R³ are halo, and R⁶ is substituted pyridyl.

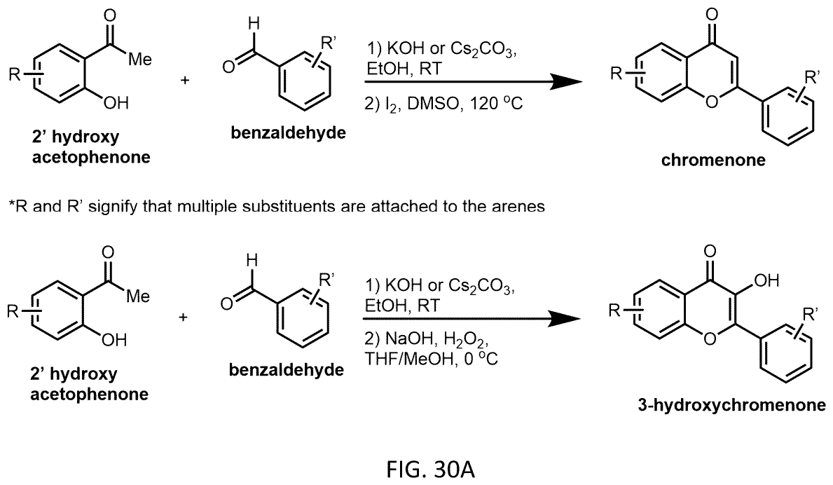

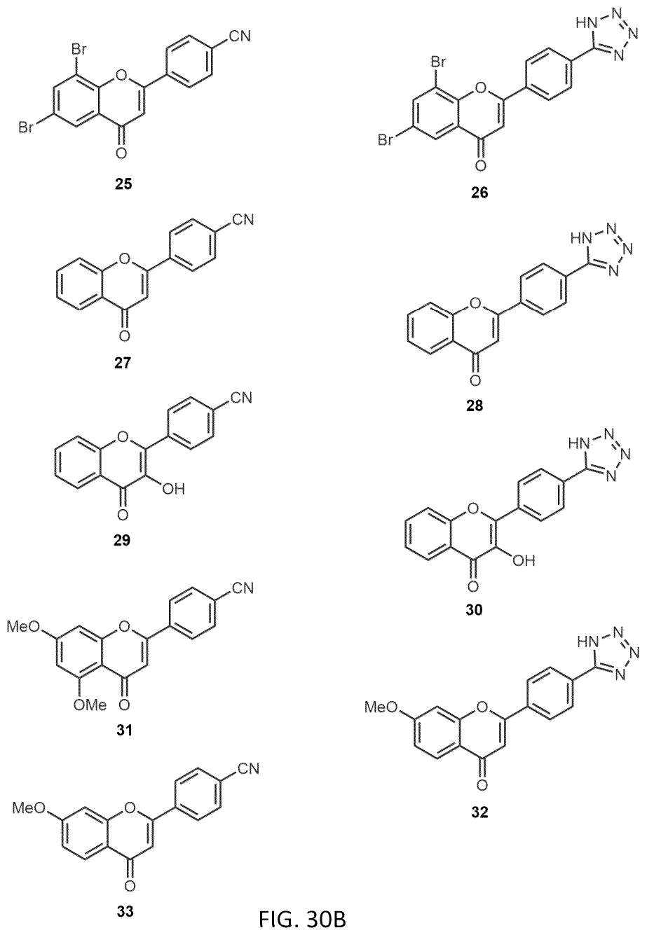

In general, combination of the appropriate 2′-hydroxyacetophenone and benzaldehyde will furnish a 2′-hydroxychalcone, which will then be subjected to two different modes of oxidative cyclization to generate either a chromenone or 3-hydroxychromenone. FIG. 30A shows general synthetic routes for the chromenones and 3-hydroxychromenones, along with structures of nine exemplary compounds prepared by the syntheses (FIG. 30B).

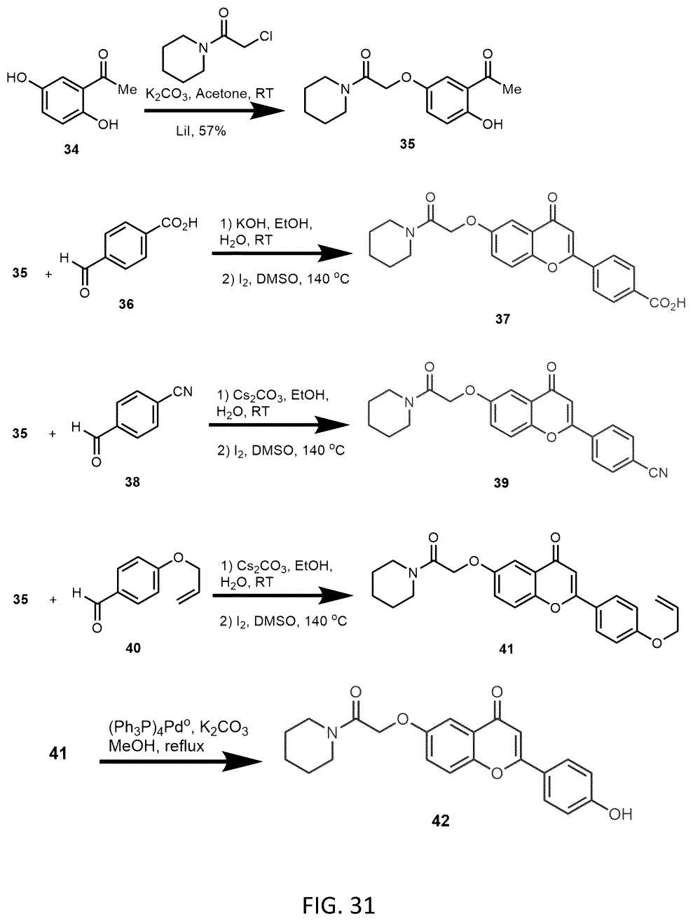

FIG. 31 shows synthetic routes for compounds 37, 39, and 42 according to Formula I, wherein R³ is

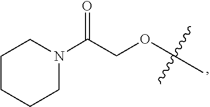 [SMILES: *OCC(=O)N1CCCCC1]

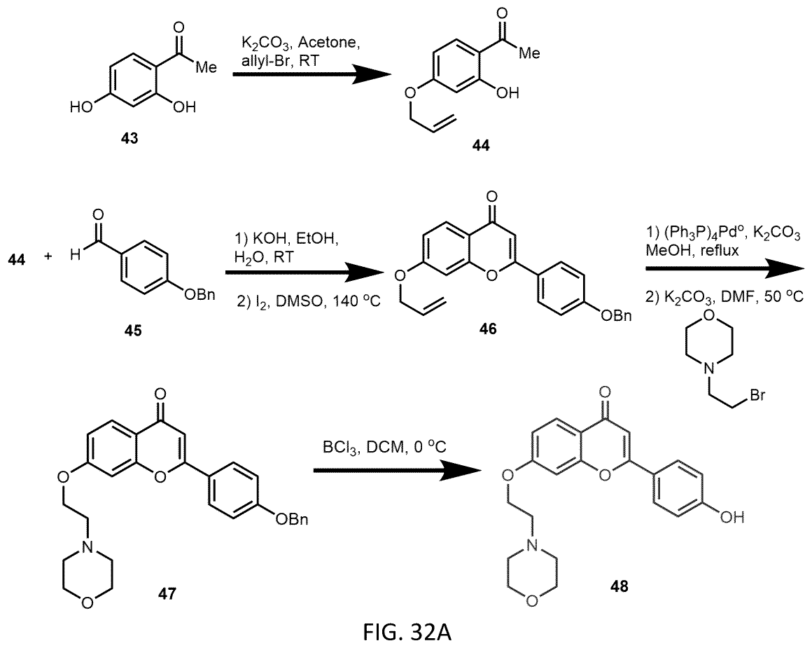

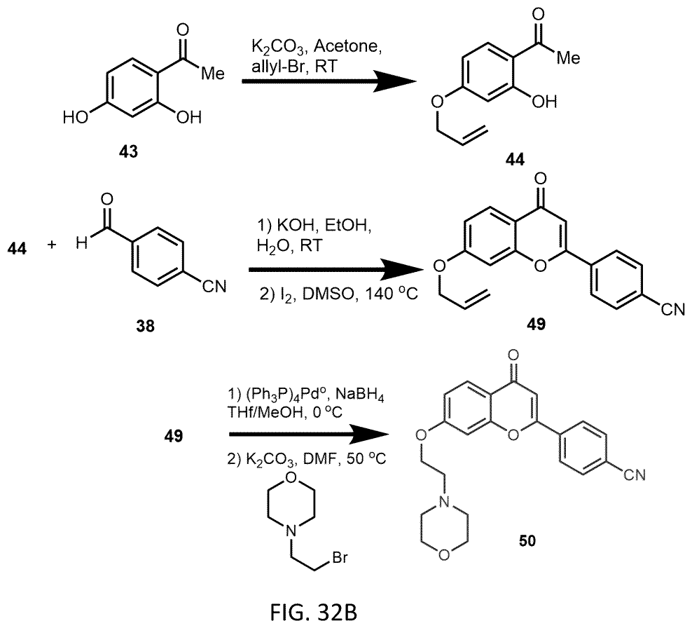

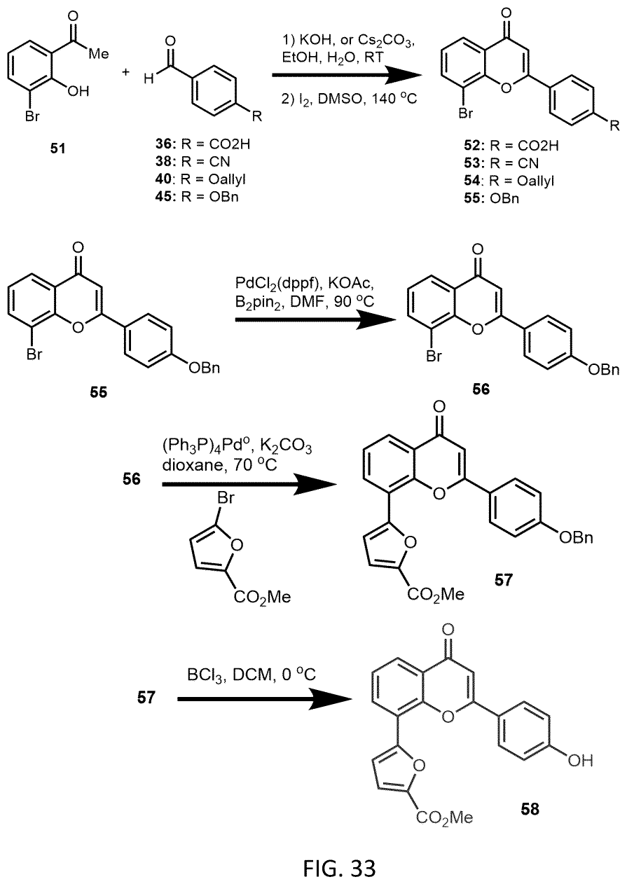

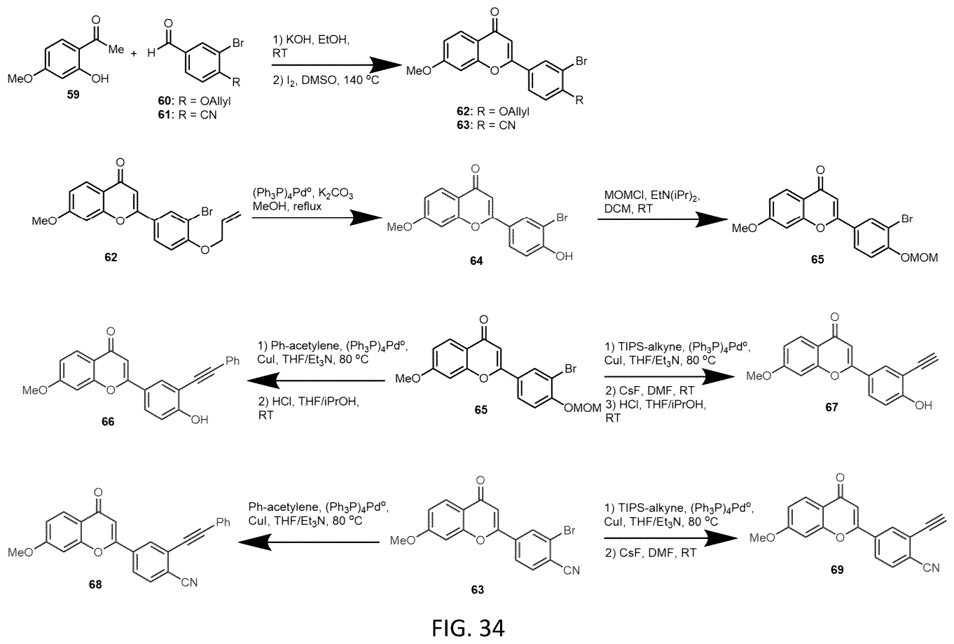

and R⁶ is substituted FIGS. 32A and 32B show a synthetic phenyl. Y route for compounds 48 and 50, respectively, according to formula I wherein R² is —O(CH₂)₂R^{a}, where R^{a} is 1,4-oxazinyl, and R⁶ is substituted phenyl. The benzoic acid analog can be similarly synthesized with the requisite aldehyde. FIG. 33 shows a synthetic route for compound 58 according to formula V. FIG. 34 shows exemplary synthetic routes for some compounds according to formula IV. With respect to the schemes in FIG. 34, compound 59 alternatively can be replaced with compounds 5, 21, or 70-74 to generate other series:

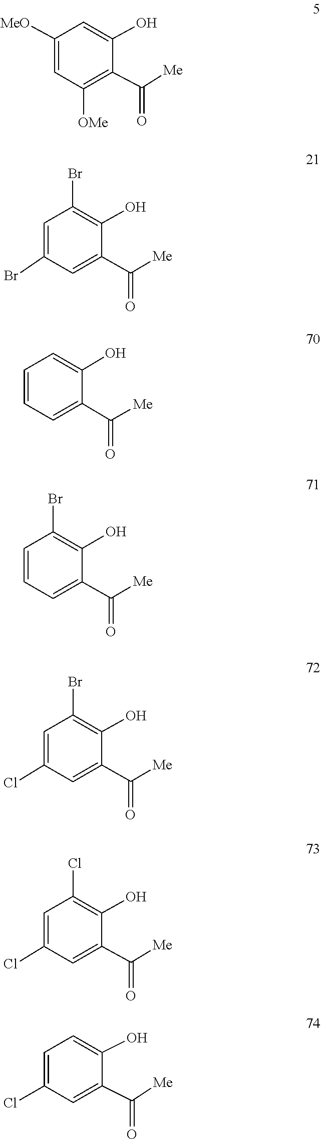

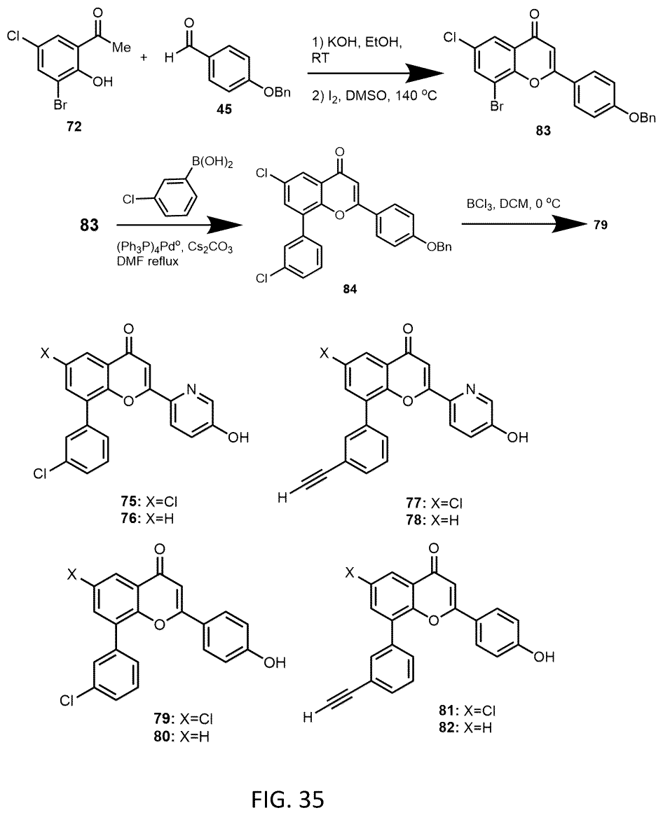

FIG. 35 shows an exemplary synthetic route for compounds 75-82 according to formula II.

Compounds that can be formed from the requisite acetophenone and aldehyde include the H-bond donor series 85-88 and the sp3 and sp-2C-linker series 89-100, among others:

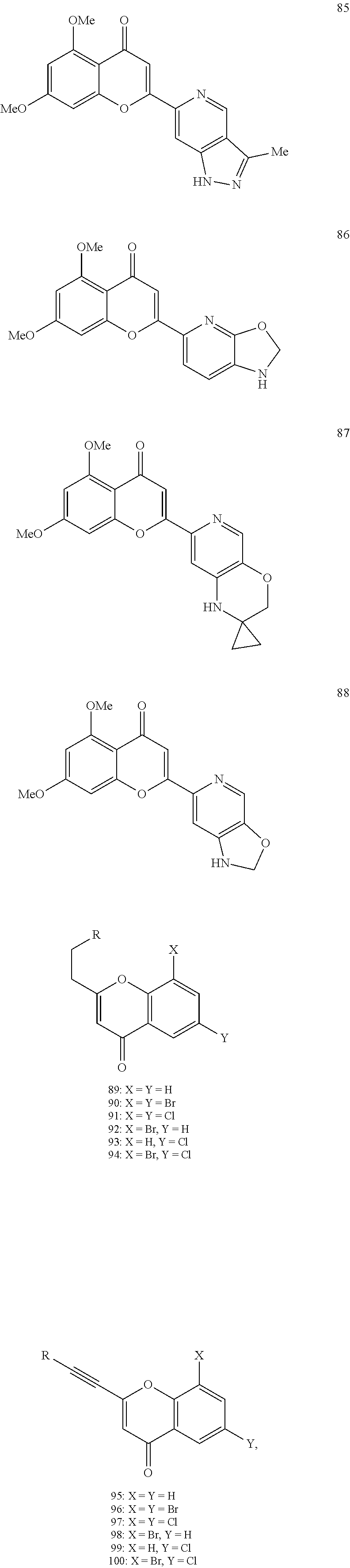

where K may be, among others:

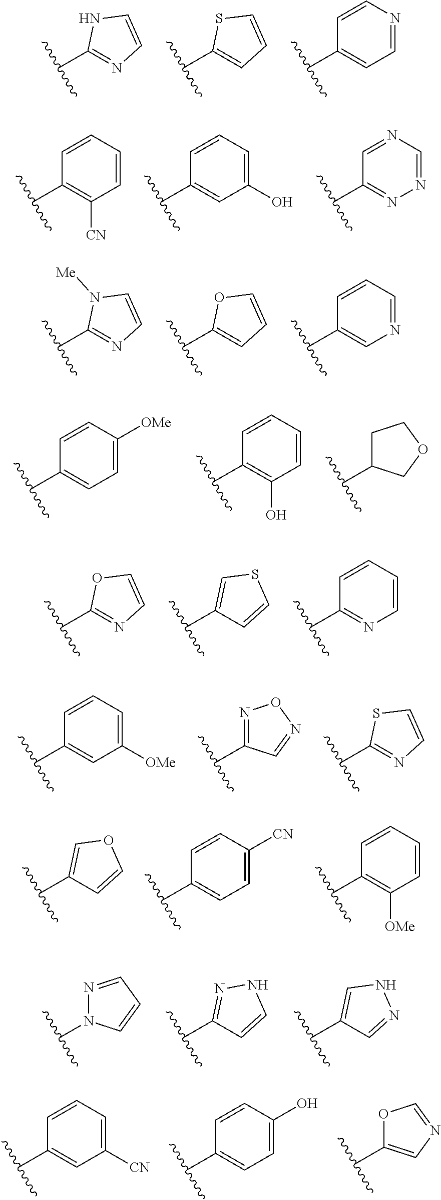

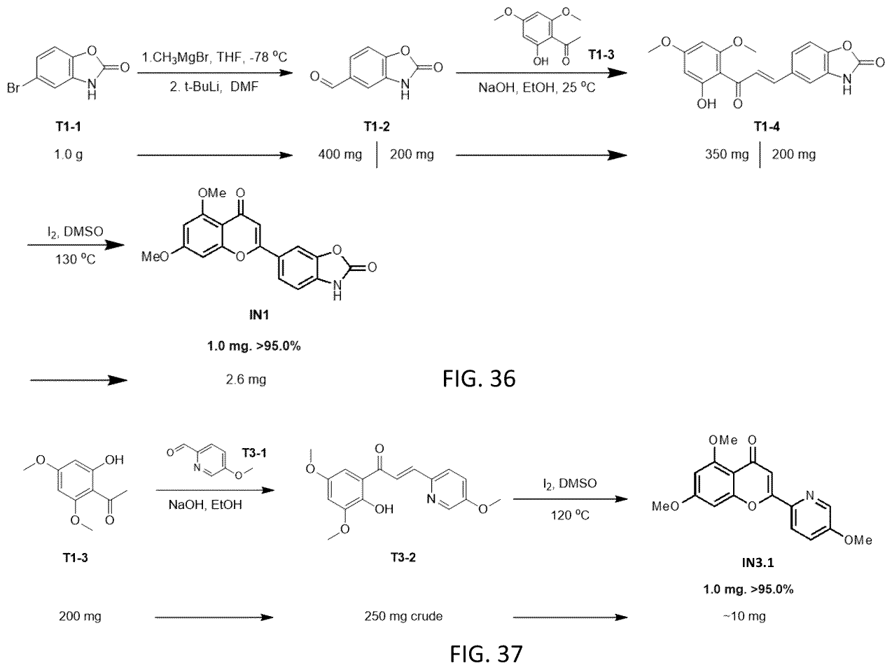

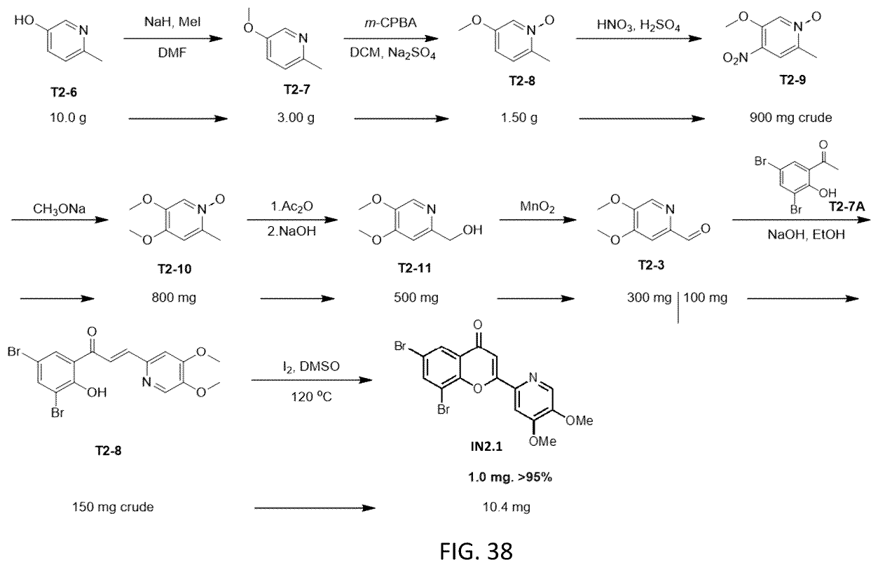

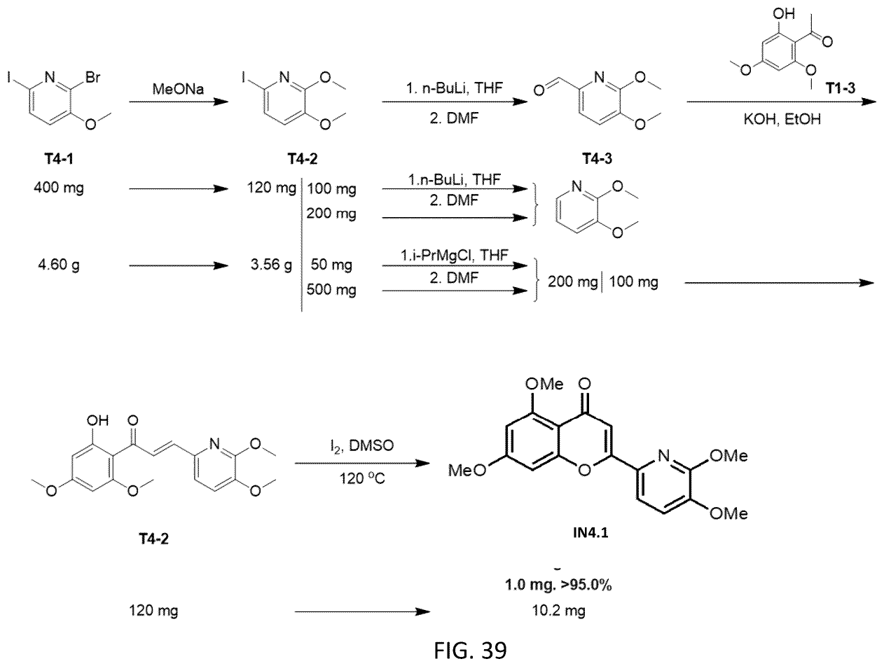

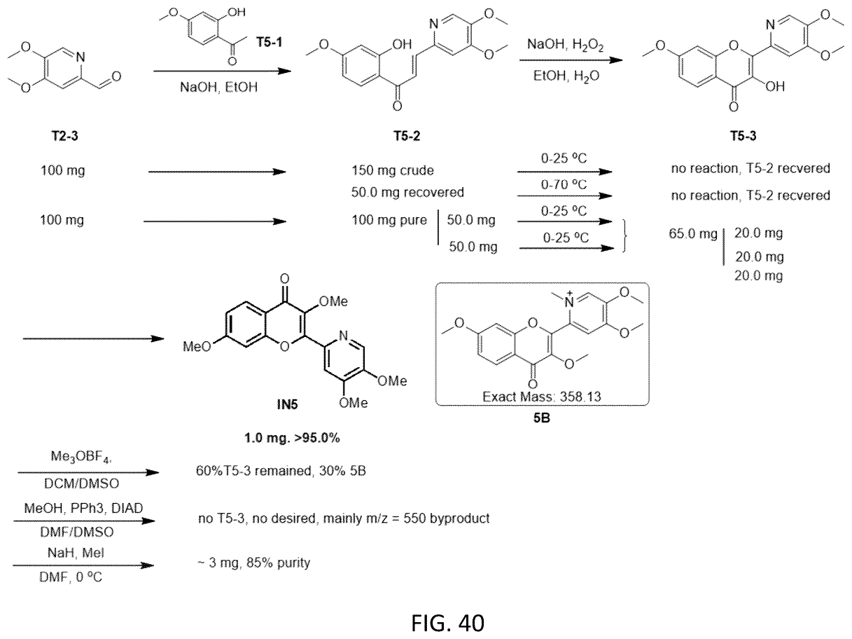

FIG. 36 shows a synthetic route for compound IN1 according to formula I where R² and R⁴ are —OR^{a}, and R⁹ and R¹⁰ together with the atoms to which they are bound form a substituted heteroaliphatic or heteroaryl ring. FIG. 37 shows a synthetic route for compound IN3.1 according to formula I where R² and R⁴ are —OR^{a}, R^{7B} is N, and R⁹ is —OR^{a}. FIG. 38 shows a synthetic route for compound IN2.1 according to formula I where R² and R⁴ are halo, R^{7B} is N, and R⁸ and R⁹ are —OR^{a}. FIG. 39 shows a synthetic route for compound IN4.1 according to formula I where R² and R⁴ are —OR^{a}, R^{7B} is N, and R⁸ and R⁹ are —OR^{a}. FIG. 40 shows a synthetic route for compound IN5 according to formula I where R² and R⁵ are —OR^{a}, R^{7B} is N, and R⁸ and R⁹ are —OR^{a}. Detailed synthetic procedures and characterization data for compound IN2.2 according to formula I are provided in Example 4, corresponding with FIG. 29.

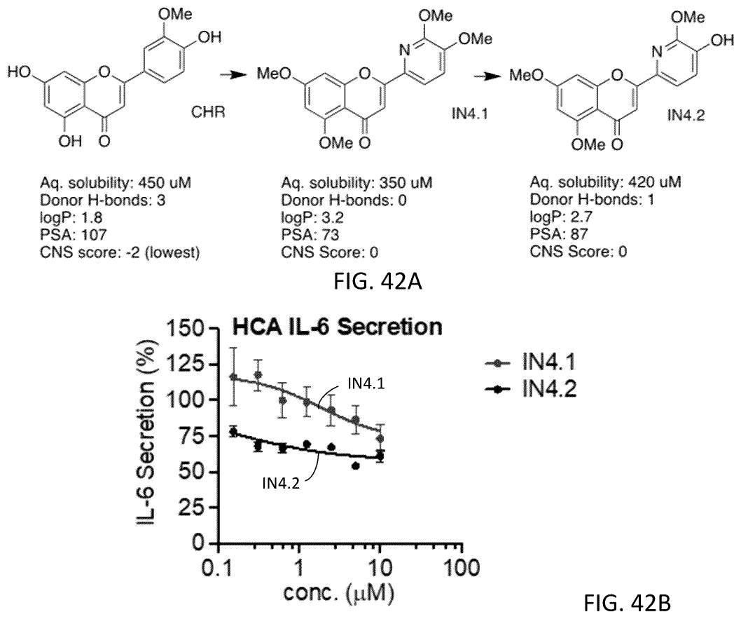

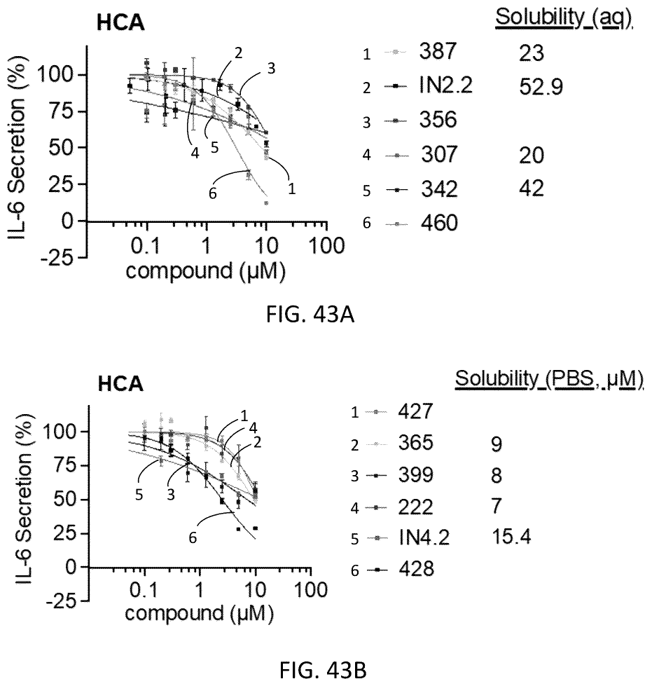

Several of the NCEs show anti-inflammatory activity comparable to that of natural flavones (FIG. 41) but with improved physicochemical properties (FIGS. 42-43). The results show that synthetic compounds without multiple metabolically labile hydroxyl moieties retain drug-like solubility, improve CNS permeability scores, and exhibit anti-inflammatory activity.

Biological activity is expected to be strongly correlated with in vitro CK2 inhibitory activity, as seen for several natural flavones (FIG. 41). Testing will show low cytotoxicity in astrocytes at <10 μM doses.

The NCEs will be evaluated for anti-inflammatory activity in cells. Dose response curves will be generated for each analogue using human astrocytes. Cells will be treated+/−IL1-β and 8 concentrations of each compound or vehicle for 5 hours, then Promega Lumit kits will be used to measure IL-6 secretion in media by luminescence quantified in a multi-mode microplate reader. More potent compounds will exhibit a lower IC₅₀ value for inhibition of luminescence/IL-6 secretion. Other pro-inflammatory cytokines such as IL-8 or TNF-α could be measured in the dose-response assays. Alternatively, the NCEs will be evaluated for anti-inflammatory activity in PBMCs. Dose response curves for inhibition of secretion of several pro-inflammatory cytokines (e.g. IL-6, IL-8, TNF-α) will be measured for each analog using Luminex or Lumit (Promega) kits in LPS-stimulated PBMCs.

The NCEs will be evaluated for in vitro activity (CK2A1/2 kinase activity assays). Dose response curves will be generated for each analogue using recombinant CK2A1 and CK2A2 kinases, peptide substrate RRRADDSDDDDD, and (γ-³²P)ATP. More potent compounds will exhibit lower IC₅₀ values for incorporation of radioactive phosphate in the substrate peptide.

The NCEs also will be evaluated for aqueous kinetic solubility. Solubility will be evaluated in buffered aqueous solutions for p.o. delivery and isotonic solutions for i.p. delivery. Better compounds will show solubilities of >10 μM, such as >50 μM, >100 μM, >300 μM, >400 μM, or even >500 μM.

The BBB permeability will be assessed in an organoid model. qRT-PCR will be used to assess and quantify compound permeability in cross-sections of a BBB organoid model (Cho et al., Nat Commun 2017, 8:15623). Better compounds will show reduction of IL-6 and IL-8 mRNA, as they are able to permeate inside the astrocytic core of the organoids and have an anti-inflammatory effect.

Selected NCEs with tolerated doses showing >50% target engagement in mice will be identified. Intraperitoneal injection (i.p.) and oral gavage (p.o.) will be tested as routes of administration, and three doses will be tested for PD and PK measures for each compound. Tolerability in healthy C57Bl/6J mice via daily treatment for 14 days will be tested. In parallel, promising leads will be evaluated a) in human liver microsomes (HLM) to assess stability and metabolism and b) kinome selectivity profiling (KINOMEscan). Desirable results will include >50% target engagement in brain tissue as measured by kinase activity in brain lysates; b) a half-life of at least t_{1/2}=˜10 hours, which is suitable for once daily dosing; c) dose exhibiting <15% weight loss over 2-week period, no mortality or morbidity; d) <50% metabolism after 1 h; e) better kinase selectivity than commercial CK2 inhibitor silmitasertib (<28/468 kinases inhibited >90% at 1 μM).

Selected NCEs will be evaluated by treating 3 BALB/c mice with one of 3 doses of compound (30, 10, and 1 mg/kg) i.p. and p.o. Blood will be collected via retro-orbital bleeding at t=0, 0.4, 1, 2, 4, 8, and 24 hr. At 24 hr, mice will be sacrificed and kidney, liver, and brain tissue will be collected. Drug and metabolite concentrations in blood will be measured via LC-MS/MS, acquiring PK parameters CLint, Vss, t_{1/2}, AUC and F (%, for p.o. administration). At the time of sacrifice, target engagement in brain and in the liver will be measured via direct quantification of CK2 activity in tissue lysates using the Cyclex CK2 ELISA assay or Western blotting of CK2 substrates such as NF-KB pS529 or other CK2 biomarkers.

Some NCEs will be evaluated for maximum tolerated dosing in mice. Tolerability will be assessed by treating healthy C57Bl/6J mice for 14 days and measuring weight daily. Dosing at 30, 10, and 1 mg/kg will be assessed. At study conclusion, complete blood count (CBC), blood chemistry, and target engagement will be assessed. Tolerated and effective dosage will be assessed by measuring target engagement in liver and brain tissue lysates, as described above. Maximum well-tolerated doses will be selected by the following criteria: <15% weight loss; no blood, hepatic or kidney toxicity, as measured by abnormal CBC and blood chemistry (e.g. blood urea nitrogen, alanine aminotransferase, aspartate aminotransferase, alkaline phosphatase, and total bilirubin); no signs of morbidity (hunched posture, rough coat, dehydration, hypothermia, lethargy, distended abdomen, signs of central nervous system toxicity such as head tilt, tremors, circling, spasticity, paralysis, unresponsive or moribund condition).

Some compounds will be tested for metabolic stability via human liver microsomes (HLM) and kinase selectivity via selectivity profiling using DiscoverX's KINOMEscan scanMAX platform, which tests 468 human kinases. Desired properties are compounds with fewer off-target kinases than CX-4945, which is known to inhibit 28 other kinases >90% at a testing concentration of 1 μM in this assay. In the HLM assay, the target goal is >50% compound stability after one hour. The best compound will have favorable PK/PD properties and adequate kinome selectivity and HLM stability as delineated above.

Selected NCEs will be evaluated in AD patient-derived neurons, astrocytes, and microglia and APP/PS1 mice. In cells, the effects of CK2 inhibition will be assessed on (a) activation of AD glia and (b) mitochondrial function in AD neurons (mitochondrial markers, ATP production). In mice, efficacy will be assessed after 4-week drug treatment at 6, 9, and 12 months by measuring disease burden through behavioral measures (cognitive tests, Morris Water Maze and contextual fear conditioning), biochemistry (Luminex assay 12-plex cytokine panel including Tnfα and Il1β cytokine levels; Ck2 pY255 and substrate phosphorylation NF-KB pS529, 1 kB pS32 levels by immunoblotting), and histology (Iba1/Gfap staining; Mff pS146, Mff, Lc3-II and Aco2 staining). This will show whether pharmacological manipulation of CK2 can ameliorate neuroinflammatory, metabolic, and cognitive phenotypes in AD preclinical models. Positive results will show significant reduction (e.g., at least 2/3 of measured outcomes) in the AD mouse model, as well as reduction of inflammatory cytokines in AD glia and/or mitochondrial function boost in AD neurons. The NCEs also will be assessed for antiviral activity against SARS-CoV-2.

Selected NCEs will be tested in AD and age-matched control iPSC-derived astrocytes and microglia and fibroblast-derived induced neurons (iNs). We will generate n=6 lines of AD-patient derived astrocytes and microglia and n=4 controls. The measured outcome will be dose-dependent reduction of baseline or IL1β-induced IL-6 and IL-8 secretion in AD astrocytes and microglia after 5 hours. The best NCEs will demonstrate low IC₅₀ for reduction of baseline inflammatory cytokine expression or the upregulation of inflammatory cytokines by IL1β. NCEs will also be tested for amelioration of mitochondrial phenotypes, including mitochondrial function (ATP production), autophagy/mitophagy markers LC3-II and ACO2, and mitochondrial fission marker pMFF by immunofluorescence staining (IF). For assessing effects on neuronal mitochondria, AD (n=5) or control (n=5) iNs will be treated with CK2 inhibitor or vehicle a) for 24 h, fixed and stained for mitophagy and mitochondrial fission markers (LC3-II, MFF p5146/MFF and ACO2) by immunofluorescence or b) co-treated for 24 h+/−TMRM, a cell permeable mitochondrial dye. Mitochondrial function will be read out by TMRM-mediated fluorescence of cells on a plate reader as well as the CellTiterGlo (Promega) assay for ATP production. NCE-treated iNs will show improved mitochondrial function, as measured by increased TMRM signal (orange fluorescence) and higher ATP production. NCE-treated iNs will exhibit increased levels of LC3-II, reduced levels of ACO2 (increased mitophagy) and reduced mitochondrial fission (reduced pMFF/MFF).

Efficacy of CK2 inhibition will be tested with lead NCEs in vivo a validated genetic AD mouse model through behavioral measures, biochemical studies, and histological studies using APP/PS1 mice (Jackson Labs stock number 004462), which exhibit progressively deteriorating hallmarks of neuroinflammation and cognitive deficits months. This model has been very well characterized phenotypically and exhibits upregulated CK2 activity between 3 and 6 months of age. APP/PS1 mice develop learning deficits by 6 months, plaques in the hippocampus and frontal cortex by 6 months, and gliosis by 6-9 months. The compound dosage will be that established from healthy wild-type isogenic C57BL/6J mice that afforded at least 50% target inhibition in vivo. Then, APP/PS1 mice and non-transgenic littermate controls will be treated with vehicle or the CK2 inhibitor daily for 4 weeks by i.p injection starting from 5, 8, or 11 months of age. Disease progression will be assessed at 3 time points: 6 months, 9 months and 12 months. These time points are chosen to provide an assessment of CK2 inhibition in delaying disease onset (preventing the early cognitive deficits and inflammation at 6 months) and in mitigating disease symptoms once pathology is firmly established (9 and 12 months). Cognitive function will be assessed by Morris water maze and contextual fear conditioning. The Morris Water maze is widely used to measure spatial learning in rodents because it is known to be affected by hippocampal and cortical function, and because APP/PS1 mice have been reported to show spatial memory deficits in the Morris Water Maze by 7 months of age. Published values suggest that the key outcome measure, time in the target quadrant, declines from approximately 30 s to 18 s, with a standard deviation of -10. Contextual fear conditioning is a second readout of cognitive ability. Like the Morris Water maze, contextual fear learning is a widely used and accepted indicator of hippocampal function. APP/PS1 mice have also been reported to show contextual fear learning deficits by 6 months of age Published values suggest the key outcome measure, freezing to a shock-associated context, declines by approximately 50% relative to non-transgenic control mice. Prior fear conditioning data suggests typical percent time freezing values of 40% for control mice with a standard deviation of 15%. Following cognitive tests, neuroinflammation will be assessed by histology for the inflammatory markers Iba1 and Gfap the mitochondrial markers Mff p5146, Mff, Lc3-II and Aco2 as well as by biochemistry (12-plex cytokine panel including Tnfa and Il1β cytokine levels, Ck2 pY255 and substrate phosphorylation (NF-KB pS529, 1 kB pS32) by Western blotting. For measuring Gfap and Iba1 signal by immunofluorescence, reasonable intensity values average 13 au with a standard deviation of 2.6 au. Trials will treat n=18 mice per condition per sacrifice timepoint (total 3 timepoints). This will allow at least n=15 mice per condition (WT+Vehicle, WT+CK2 inhibitor, APP/PS1+Vehicle, APP/PS1+CK2 inhibitor) in case of study drop-out due to unforeseen circumstances. All mice will complete both Morris Water maze and contextual fear conditioning tests. At the conclusion of behavioral testing, mice will be saline-perfused. One half of each brain will be immediately flash frozen on dry ice and reserved for biochemical assays. The other half will be drop-fixed in paraformaldehyde for immunohistochemistry. Alternatively, osmotic pumps could be used for chronic continuous delivery directly into the brain by i.c.v. cannula. The cannula would be removed 1 week before doing the behavior studies to allow healing. In that case, the mice would be tested on the Barnes maze instead of the water maze.

### Example 4

### Supporting Information for the Synthesis of IN2.2 According to FIG. 29

The following procedures and data display the details of the synthetic approach delineated in FIG. 29 as a representative example.

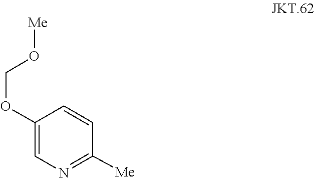 [SMILES: COCOc1ccc(C)nc1]

MOMCl (4.55 mL, 60 mmol, 1.2 eq) was added dropwise to a stirring solution of 3-hydroxy-6-methyl pyridine (12, 5.45 g, 50 mmol, 1 eq) and ethyl-diisopropylamine (9.60 mL, 55 mmol, 1.1 eq) in dry dichloromethane (250 mL) at RT. The reaction was then stirred at room temperature.

After 16 h, the reaction was washed with saturated aqueous sodium bicarbonate and brine, dried over sodium sulfate, filtered, and concentrated at reduced pressure. The crude reaction mixture was purified by chromatography on normal phase silica gel (ethyl acetate/hexane eluent) to give JKT.62 (6.65 g, 43.5 mmol, 87%) as a colorless liquid.

¹H NMR (500 MHz, CDCl₃): δ: 8.29 (d, 1H), 7.27 (dd, 1H), 7.07 (d, 1H), 5.16 (s, 2H), 3.47 (s, 3H), 2.50 (s, 3H).

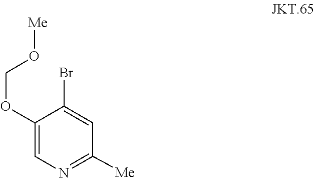 [SMILES: COCOc1cnc(C)cc1Br]

To a stirring solution of JKT.62 (1.53 g, 10 mmol, 1 eq) in in THF (40 mL) in a 200 mL recovery flask cooled in a dry ice/acetone bath was added nBuLi in hexane (1.6 M, 15.6 mL, 25 mmol, 2.5 eq), dropwise. The yellow-red solution was then maintained in the dry ice/acetone bath. 20 mL additional THF were added. Then, a solution of CBr₄ (8.29 g, 25 mmol, 2.5 eq) in THF (25 mL) was added dropwise. The reaction was allowed to warm slowly, to room temperature. After stirring for 15 h, the reaction was poured into saturated ammonium chloride aqueous and extracted twice with ethyl acetate. The combined organic fractions were washed with brine, dried over sodium sulfate, filtered, and concentrated at reduced pressure. The crude reaction mixture was purified by chromatography on normal phase silica gel (ethyl acetate/hexane eluent) to give JKT.65 (1.49 g, 6.4 mmol, 64%) as a brown liquid.

¹H NMR (500 MHz, CDCl₃): δ: 8.20 (s, 1H), 7.15 (s, 1H), 5.13 (s, 2H), 3.43 (s, 3H), 2.37 (s, 3H).

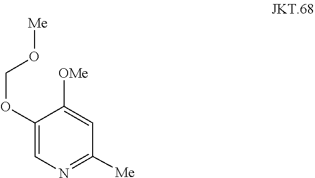 [SMILES: COCOc1cnc(C)cc1OC]

A mixture of JKT.65 (2.76 g, 11.9 mmol, 1 eq) and NaOMe (25 weight % in methanol, 5.5 mL, 23.8 mmol, 2 eq) in dimethylformamide (24 mL) was heated to 75° C. with stirring for 15 h. The resultant mixture was cooled to room temperature, poured into water, and extracted thrice with ethyl acetate. The combined organic fractions were washed with water and brine, dried over sodium sulfate, filtered, and concentrated at reduced pressure. The crude reaction mixture was purified by chromatography on normal phase silica gel (ethyl acetate/hexane eluent) to give JKT.68 (950 mg, 5.2 mmol, 44%) as a brown liquid.

¹H NMR (500 MHz, CDCl₃): δ: 8.20 (s, 1H), 6.68 (s, 1H), 5.17 (s, 2H), 3.89 (s, 3H) 3.51 (s, 3H), 2.48 (s, 3H).

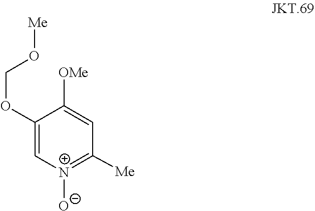 [SMILES: COCOc1c[n+]([O-])c(C)cc1OC]

A solution of the JKT.68 (950 mg, 5.2 mmol, 1 eq) in DCM (52 mL) was treated with mCPBA (70 wt %, 1356 mg, 5.5 mmol, 1.05 eq). The mixture was stirred at room temperature for 2 h. The reaction was then concentrated partway and purified directly by chromatography on normal phase silica gel (methylene chloride/methanol eluent) to give JKT.69 (750 mg, 3.77 mmol, 73%) as a white solid.

¹H NMR (500 MHz, CDCl₃): δ: 8.27 (s, 1H), 6.69 (s, 1H), 5.17 (s, 2H), 3.92 (s, 3H) 3.49 (s, 3H), 2.50 (s, 3H).

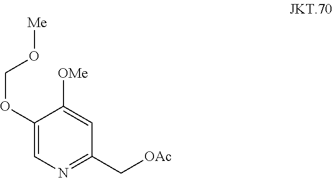 [SMILES: COCOc1cnc(COC(C)=O)cc1OC]

A solution of JKT.69 (750 mg, 3.77 mmol, 1 eq) and ethyl-diisopropylamine (7.3 mL, 41.5 mmol, 11 eq) in 1,4-dioxane (7.6 mL) was treated with acetic anhydride (3.6 ml, 37.7 mmol 10 eq). The resultant mixture was heated with stirring to 110° C. for 15 h. The reaction was then cooled to room temperature, poured into water and extracted thrice with methylene chloride. The combined organic fractions were dried over sodium sulfate, filtered, and concentrated at reduced pressure. The crude reaction mixture was purified by chromatography on normal phase silica gel (ethyl acetate/hexane eluent) to give JKT.70 (830 mg, 3.44 mmol, 91%) as a brown oil.

¹H NMR (500 MHz, CDCl₃): δ: 8.33 (s, 1H), 6.92 (s, 1H), 5.22 (s, 2H), 5.14 (s, 2H), 3.94 (s, 3H) 3.52 (s, 3H), 2.04 (s, 3H).

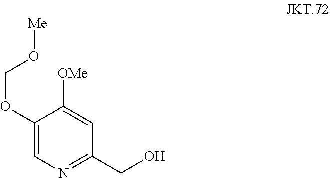 [SMILES: COCOc1cnc(CO)cc1OC]

A solution of JKT.70 (830 mg, 3.44 mmol, 1 eq) in methanol (34 mL) was treated with potassium carbonate (499 mg, 3.61 mmol, 1.05 eq), and the resultant mixture was stirred at room temperature for 1 h. The reaction was then concentrated at reduced pressure. It was then partitioned between water and methylene chloride. The aqueous layer was extracted twice more with methylene chloride. The combined organic fractions were dried over sodium sulfate, filtered, and concentrated at reduced pressure to give JKT.72 (480 mg, 2.41 mmol, 70%) as an off-white solid. The crude material was suitable for use in the next reaction.

¹H NMR (500 MHz, CDCl₃): δ: 8.29 (s, 1H), 6.79 (s, 1H), 5.21 (s, 2H), 4.68 (s, 2H), 3.92 (s, 3H) 3.53 (s, 3H).

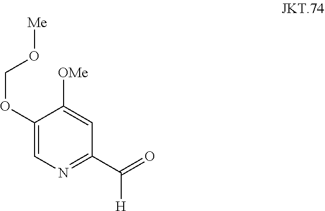 [SMILES: COCOc1cnc(C=O)cc1OC]

A solution of JKT.72 (398 mg, 2 mmol, 1 eq) in 5:1 methylene chloride: dimethylsulfoxide (40 mL) was treated with Dess-Martin periodinane (933 mg, 2.2 mmol, 1.1 eq). The mixture was stirred at room temperature for 1 h. The reaction was then poured into saturated sodium thiosulfate aqueous, and the layers were separated. The aqueous layer was extracted twice more with methylene chloride. The combined organic fractions were dried over sodium sulfate, filtered, and concentrated at reduced pressure. The crude reaction mixture was purified by chromatography on normal phase silica gel (ethyl acetate/hexane eluent) to give JKT.74 (342 mg, 1.74 mmol, 87%) as a white solid.

¹H NMR (500 MHz, CDCl₃): δ: 9.95 (s, 1H), 8.52 (s, 1H), 7.54 (s, 1H), 5.34 (s, 2H), 4.00 (s, 3H) 3.54 (s, 3H).

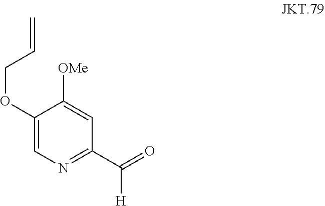

A solution of JKT.74 (630 mg, 3.2 mmol, 1 eq) in formic acid (32 mL) was heated to 60° C. for 1 h. The reaction was then concentrated at reduced pressure. The residue obtained was treated with cesium carbonate (10.40 g, 32 mmol, 10 eq) and sonicated. The mixture obtained was suspended in dimethylformamide (32 mL) and stirred vigorously. Allyl bromide (0.56 mL, 6.4 mmol, 2 eq) was added, and the resultant mixture was stirred at room temperature for 17 h. The reaction was poured into water and ethyl acetate, and the layers were separated. The aqueous layer was extracted twice more with ethyl acetate. The combined organic fractions were washed with brine, dried over sodium sulfate, filtered, and concentrated at reduced pressure. The crude reaction mixture was purified by chromatography on normal phase silica gel (ethyl acetate/hexane eluent) to give JKT.79 (200 mg, 1.04 mmol, 33%) as waxy, off-white solid.

¹H NMR (500 MHz, CDCl₃): δ: 9.93 (s, 1H), 8.27 (s, 1H), 7.52 (s, 1H), 6.08 (m, 1H), 5.47 (m, 1H), 5.38 (m, 1H), 4.77 (m, 2H) 4.00 (s, 3H).

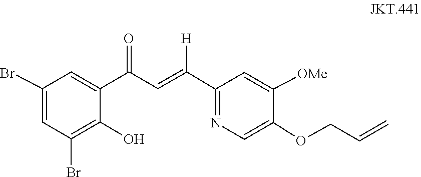 [SMILES: C=CCOc1cnc(/C=C/C(=O)c2cc(Br)cc(Br)c2O)cc1OC]

A mixture of acetophenone 21 (442 mg, 1.5 mmol, 1 eq) and JKT.79 (290 mg, 1.5 mmol, 1 eq) in ethanol (7.2 mL) was treated with KOH (3M in 96% aqueous ethanol, 2 mL, 6 mmol, 4 eq). The resultant orange slurry was stirred at room temperature vigorously for 17 h. The reaction was then quenched with 6 M HCl, leading to the formation of a yellow solid, which was isolated on a filter frit. This crude material was recrystallized from minimal boiling methanol to give JKT.441 (484 mg, 1.03 mmol, 69%) as a yellow solid.

¹H NMR (500 MHz, DMSO): δ: 12.90 (s, 1H), 8.40 (s, 1H), 8.37 (s, 1H), 8.26 (m, 1H), 8.17 (s, 1H), 7.99 (m, 1H), 7.81 (m, 1H), 6.06 (m, 1H), 5.44 (m, 1H), 5.32 (m, 1H), 4.78 (m, 2H) 4.05 (s, 3H).

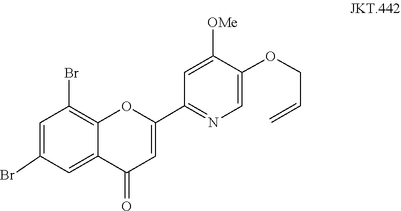 [SMILES: C=CCOc1cnc(-c2cc(=O)c3cc(Br)cc(Br)c3o2)cc1OC]

JKT.441 (469 mg, 1 mmol, 1 eq) was solvated in 20 mL of DMSO at 70° C. After addition of iodine (26 mg, 0.1 mmol, 0.1 eq), the reaction vessel was sealed and heated with stirring to 140° C. for 3 h. The reaction was cooled to room temperature and poured into water (100 mL), followed by extraction with methylene chloride (3×50 mL). The combined organic fractions were washed with water and then 0.1% Na₂S₂O₃, dried over sodium sulfate, filtered, and concentrated at reduced pressure. The residue obtained was triturated in ca 10 mL boiling methanol and filtered. The filtrate was concentrated at reduced pressure to give JKT.442 (170 mg, 0.36 mmol, 36%) as a tan solid.

¹H NMR (500 MHz, CDCl₃): δ: 8.32 (d, 1H), 8.27 (s, 1H), 8.05 (d, 1H), 7.81 (s, 1H), 7.40 (s, 1H), 6.09 (m, 1H), 5.48 (m, 1H), 5.39 (m, 1H), 4.78 (m, 2H) 4.06 (s, 3H).

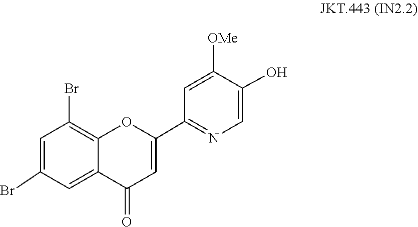 [SMILES: COc1cc(-c2cc(=O)c3cc(Br)cc(Br)c3o2)ncc1O]

A mixture of JKT.442 (140 mg, 0.3 mmol, 1 eq), tetrakis(triphenylphosphine)-palladium(0) (18 mg, 0.015 mmol, 0.05 eq), and potassium carbonate (166 mg, 1.2 mmol, 4 eq) in methanol (3 mL) in a dry 25 mL flask was sparged with argon for two minutes. The vessel was sealed and heated with stirring to reflux. The reaction was cooled to room temperature after two hours. The reaction was crashed out with saturated ammonium chloride (ca 8 mL), and the resulting solid was isolated on a filter frit and washed with water. The solid was then triturated in minimal boiling methanol and filtered once more to give JKT.443 (60 mg, 0.14 mmol, 47%) as a tan solid.

¹H NMR (500 MHz, DMSO): δ: 10.50 (br s, 1H), 8.41 (d, 1H), 8.20 (s, 1H), 8.08 (d, 1H), 7.76 (s, 1H), 7.09 (s, 1H), 3.98 (s, 3H).

MS (ESI): Predicted: 425.90. Found: 425.80.

### Example 5

### IL-6 Reduction in Astrocytes

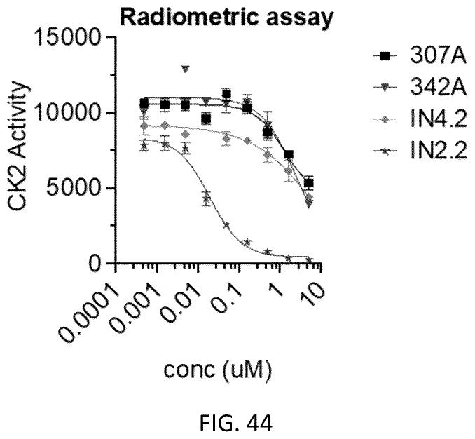

Human primary astrocytes (n=3 patients) were treated with compounds 387, IN2.2, 356, 307, 342, 427, 365, 399, 222, 428, 460, and IN4.2 (Table 5) to determine dose-dependent reduction of IL-6 levels in primary astrocytes (mean±s.e.m, n=3). Aqueous solubility (PBS, pH=7.4) was also determined for a subset of the compounds (Eurofins). The results are shown in FIGS. 43A and 43B. FIG. 44 shows dose-dependent inhibition of CK2A1 activity by four of the compounds in vitro (Eurofins KinaseProfiler radiometric kinase assay).

TABLE 5

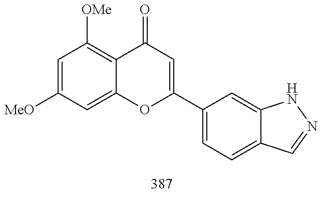 [SMILES: COc1cc(OC)c2c(=O)cc(-c3ccc4cn[nH]c4c3)oc2c1]

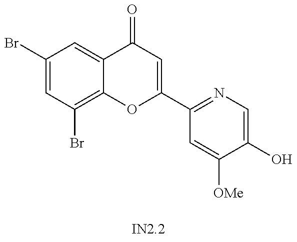 [SMILES: COc1cc(-c2cc(=O)c3cc(Br)cc(Br)c3o2)ncc1O]

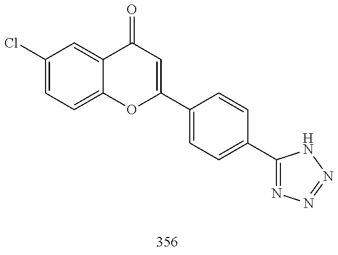 [SMILES: O=c1cc(-c2ccc(-c3nnn[nH]3)cc2)oc2ccc(Cl)cc12]

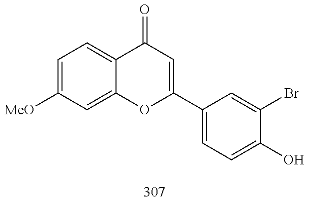 [SMILES: COc1ccc2c(=O)cc(-c3ccc(O)c(Br)c3)oc2c1]

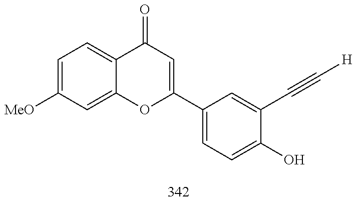 [SMILES: C#Cc1cc(-c2cc(=O)c3ccc(OC)cc3o2)ccc1O]

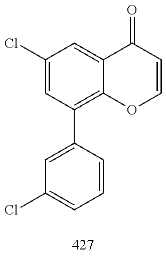 [SMILES: O=c1ccoc2c(-c3cccc(Cl)c3)cc(Cl)cc12]

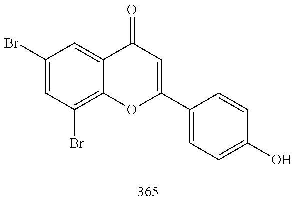 [SMILES: O=c1cc(-c2ccc(O)cc2)oc2c(Br)cc(Br)cc12]

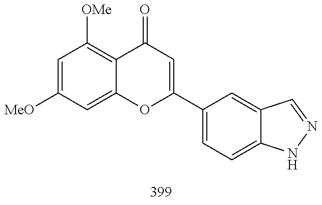 [SMILES: COc1cc(OC)c2c(=O)cc(-c3ccc4[nH]ncc4c3)oc2c1]

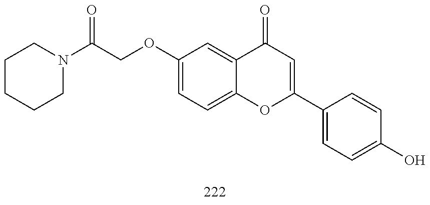 [SMILES: O=C(COc1ccc2oc(-c3ccc(O)cc3)cc(=O)c2c1)N1CCCCC1]

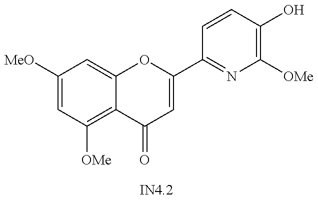 [SMILES: COc1cc(OC)c2c(=O)cc(-c3ccc(O)c(OC)n3)oc2c1]

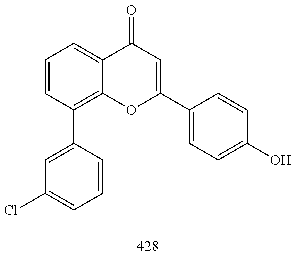 [SMILES: O=c1cc(-c2ccc(O)cc2)oc2c(-c3cccc(Cl)c3)cccc12]

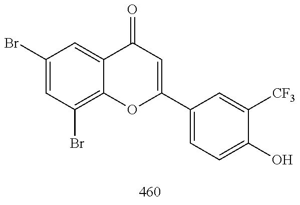 [SMILES: O=c1cc(-c2ccc(O)c(C(F)(F)F)c2)oc2c(Br)cc(Br)cc12]

### Example 6

### Antiviral Activity Against SARS-CoV-2

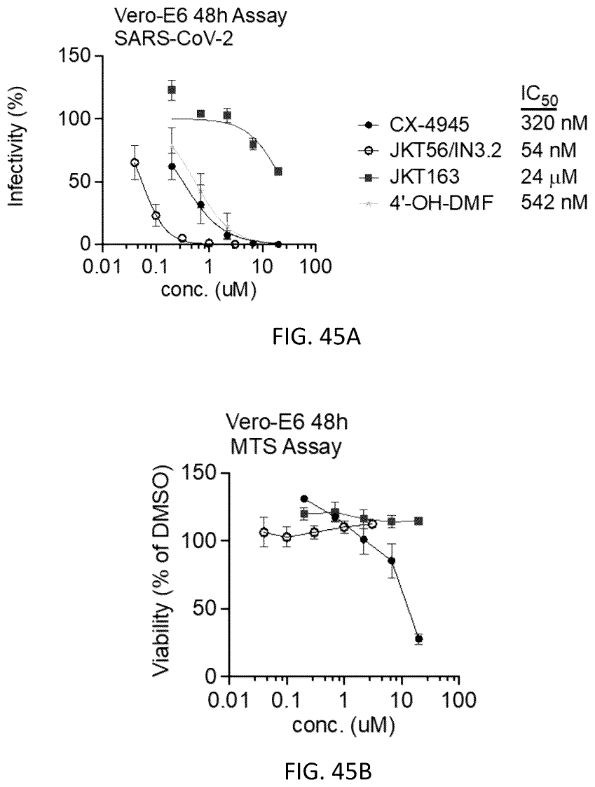

Vero-E6 cells were plated at 2,500 cells/well in DMEM+10% FBS. 18 hours later, media was replaced by the compounds in Table 6 at 1.1× concentration (0.1% DMSO) in DMEM+2% FBS. 2 h later, 10 uL of SARS-CoV-2 virus was added at MOI=0.025 and the cells were incubated for 48 h. Cells were fixed in 4% PFA, washed, and stained with anti-NP (nucleocapsid protein) antibody and Alexa-Fluor 594 secondary antibody as well as SytoxGreen nucleic acid stain. Infectivity was determined by the percentage of NP-positive cells relative to total cells in DMSO controls as quantified by immunofluorescence on a Nikon microscope (mean±s.e.m., n=3) (FIG. 44A). In parallel, Vero-E6 cells were exposed to compounds in DMEM+2% FBS for 48 h, and dose-response curves for viability were determined by the MTS Assay (Promega CellTiter-96 AQ One, mean±s.e.m., n=3) (FIG. 45B).

TABLE 6

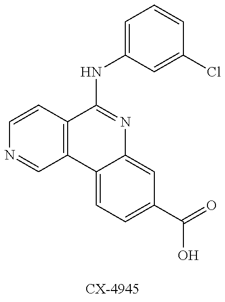 [SMILES: O=C(O)c1ccc2c(c1)nc(Nc1cccc(Cl)c1)c1ccncc12]

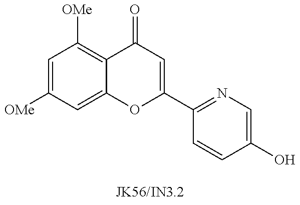 [SMILES: COc1cc(OC)c2c(=O)cc(-c3ccc(O)cn3)oc2c1]

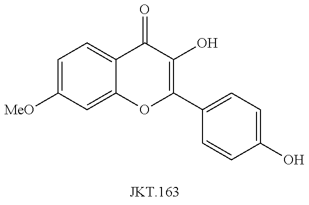 [SMILES: COc1ccc2c(=O)c(O)c(-c3ccc(O)cc3)oc2c1]

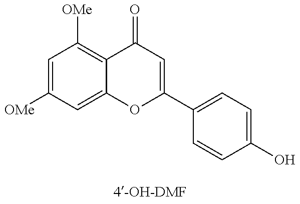 [SMILES: COc1cc(OC)c2c(=O)cc(-c3ccc(O)cc3)oc2c1]

### Serum/Tissue Distribution and Blood-Brain Barrier Permeability

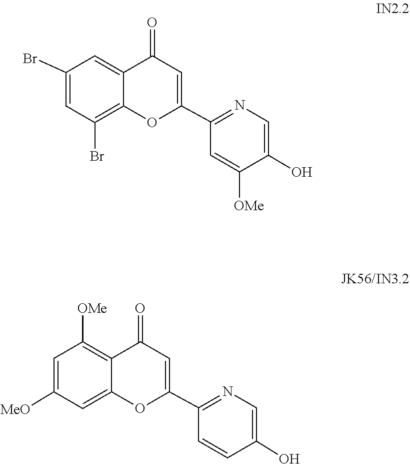 [SMILES: COc1cc(-c2cc(=O)c3cc(Br)cc(Br)c3o2)ncc1O ; COc1cc(OC)c2c(=O)cc(-c3ccc(O)cn3)oc2c1]

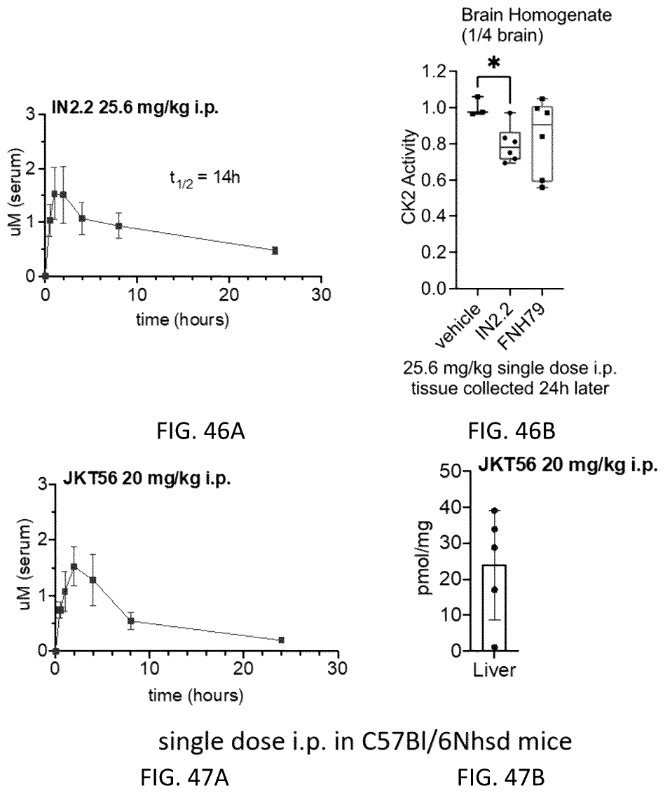

IN2.2 or vehicle (3% wt/wt hydroxpropymethylcellulose aqueous solution) was injected as a single dose (25.6 mg/kg) i.p. in 2-3 month old C57Bl/6Nhsd mice (n=6 and n=3 controls). Blood was collected at t=0, 0.5, 1, 2, 4, 8, 24 via retro-orbital bleeding and allowed to clot at room temperature to separate serum and clarified. At the final timepoint, mice were anesthetized with ketamine and tissues were harvested. Drug concentrations in serum and tissues were determined by LC/MS-MS. Target engagement in the brain was determined in brain homogenates from treated animals using the Cyclex CK2 ELISA kit. IN2.2 showed superior target engagement in brain in comparison to related compound FNH79 (boxplot, two-tailed t-test, p<0.05). Results are shown in FIGS. 46A and 46B.

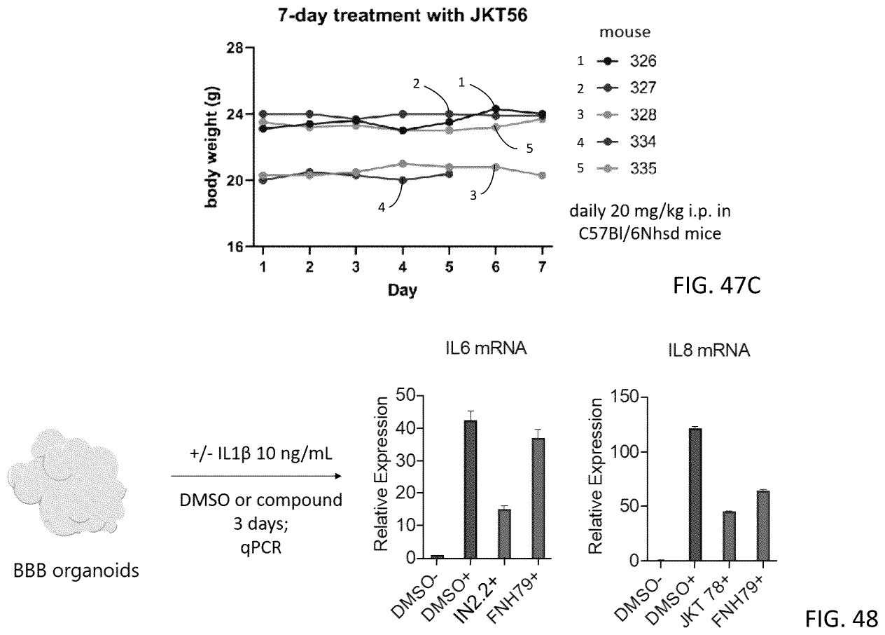

JKT56/IN3.2 or vehicle (2% wt/wt hydroxpropymethylcellulose aqueous solution) was injected as a single dose (20 mg/kg) i.p. in 2-3 month old C57Bl/6Nhsd mice (n=6). Blood was collected at t=0, 0.5, 1, 2, 4, 8, 24 via retro-orbital bleeding and allowed to clot at room temperature to separate serum and clarified. At the final timepoint, mice were anesthetized with ketamine and tissues were harvested. Drug concentrations in serum and tissues were determined by LC/MS-MS (FIGS. 47A and 47B). Tolerability was assessed in 2-3 month old C57Bl/6Nhsd mice (mean±s.d., n=5) by injecting with JKT56/IN3.2 daily at 20 mg/kg and monitoring closely for any signs of toxicity based on body weight changes and behavior/appearance (e.g. signs of distress, rough coat, squinty eyes, hunched posture). All mice appeared to be healthy for the duration of the study and their body weights were stable (FIG. 47C). One mouse was found deceased on day 6.

BBB permeability of IN2.2 was also determined in BBB organoids (Cho et al., Nat Commun 2017, 8:15623). Pools of -50 organoids were treated for 3 days with IL1-β and DMSO or compounds (FNH79 at 20 μM, IN2.2 at 6.7 μM) and collected in RNA-Bee reagent (Amsbio). qPCR for IL6 and IL8 expression was performed (mean±s.d., n=3). IN2.2 showed superior permeability and anti-inflammatory activity as shown by reduction of IL6 and IL8 expression compared to related compound FNH79 (FIG. 48).

### Example 8

### Therapeutic Uses

A subject identified as having a disease or conditioned characterized at least in part by dysregulated CK2 activity is administered a therapeutically effective amount of a pharmaceutical composition comprising a CK2 inhibitor as disclosed herein. In some embodiments, the subject is identified as having a disease or condition characterized at least in part by inflammation, such as neuroinflammation. In some examples, the subject is identified as having cancer, cardiac hypertrophy, multiple sclerosis, cystic fibrosis, a neurodegenerative disease, bipolar disorder, a viral infection, or any combination thereof. In one example, the subject has cancer and is identified as having a mutated CSNK2A1 genotype and/or upregulated levels of CK2. In another example, the subject has Parkinson's disease, Alzheimer's disease, or bipolar disorder. In yet another example, the subject has a viral infection, such as a SARS-CoV-2 infection. In any of the foregoing examples, the subject may be administered the therapeutically effective amount of the pharmaceutical composition at periodic intervals for an effective period of time to mitigate at least one sign or symptom of the disease or condition. For example, the subject may be administered the therapeutically effective amount of the pharmaceutical composition once daily or in divided doses over the course of a day, such as 2-3 divided doses per day. The pharmaceutical composition is administered by any suitable route including, but not limited to, orally, parenterally (e.g., intravenously, intramuscularly, subcutaneously), or topically.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A compound according to formula I, or a pharmaceutically acceptable salt, hydrate, stereoisomer, or tautomer thereof: 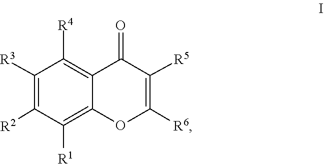
where R¹ is halo, H, substituted or unsubstituted aryl, substituted or unsubstituted heteroaryl, —SF₅, or -L-Q where L is a linker and Q is an E3-ligase binding moiety;
R² is H, —O(CH₂)ₘR^{a} where m is an integer greater than or equal to zero, or -L-Q;
R³ is halo, H, OR^{a}, 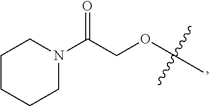 [SMILES: *OCC(=O)N1CCCCC1]
a hydrophobic group, or -L-Q;
R⁴ and R⁵ independently are H or —OR^{a};
R^{a} is substituted or unsubstituted aliphatic, H, substituted or unsubstituted heteroaliphatic, or acyl; and
R⁶ is 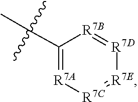
where
R^{7A} is N or CH and R^{7B} is N or CH where one or both of R^{7A} and R^{7B} is N, R^{7C} is C—R⁸, R^{7D} is C—R¹⁰, and R^{7E} is C—R⁹,
R⁸ is —OR^{a}, H, substituted or unsubstituted alkyl, or halo, R⁹ is —OR^{a}, —CN, —C(O)OR^{a}, or azole, and R¹⁰ is H, alkynyl, -alkynyl-R¹¹, -alkynyl-heteroaliphatic, halo, —OR^{a}, or a hydrophobic group, or R⁸/R⁹ or R⁹/R¹⁰ together with the atoms to which they are bound form a substituted or unsubstituted heteroaliphatic or heteroaryl ring, and
R¹¹ is substituted or unsubstituted heteroaryl or substituted or unsubstituted aryl.

2. The compound according to claim 1, wherein:
(i) R¹ and R³ are halo, and R² and R⁴ independently are H, OH, or methoxy; or
(ii) R¹ and R³ are H, and R² and R⁴ are OH or methoxy; or
(iii) R¹ and R³ are other than H, and R² and R⁴ are H; or
(iv) one of R¹-R⁴ is other than H, and the others of R¹-R⁴ are H.

3. The compound according to claim 1, where: each of Z¹-Z⁴ is
(i) R¹ and R³ independently are bromo or chloro, and R² and R⁴ are H; or
(ii) R² and R⁴ are methoxy, and R¹ and R³ are H; or
(iii) R² is methoxy and R¹, R³, and R⁴, are H; or
(iv) R² and R⁴ are H, R³ is H or halo, and R¹ is halo or 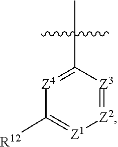
where each of Z¹-Z⁴ is CH or one of Z¹-Z⁴ is N and the others of Z¹-Z⁴ are CH; and R¹² is halo or alkynyl.

4. The compound according to claim 1, wherein:
(i) one of R⁸ and R¹⁰ is H and the other of R⁸ and R¹⁰ is methoxy, halo, alkynyl, or substituted or unsubstituted alkyl; or
(ii) R⁸ and R¹⁰ are H; or
(iii) R⁹ is hydroxy, methoxy, —CO₂H, or cyano; or
(iv) R⁶ is

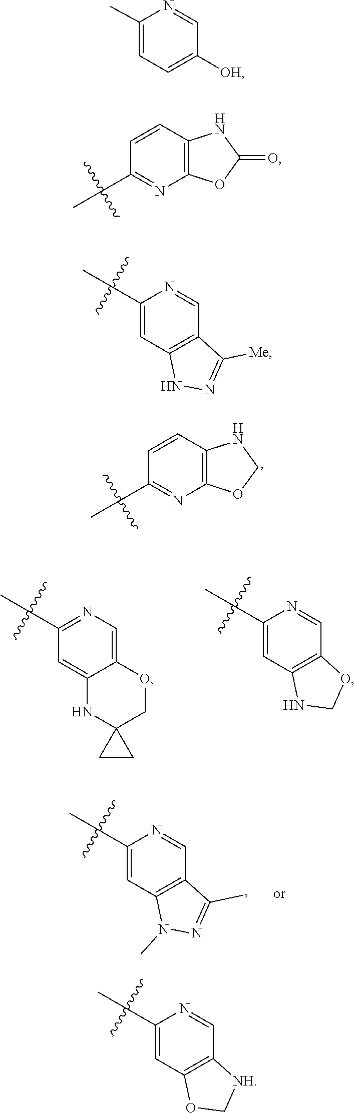

5. The compound of claim 1, having a structure according to formula II or formula III, or a pharmaceutically acceptable salt, hydrate, stereoisomer, or tautomer thereof: 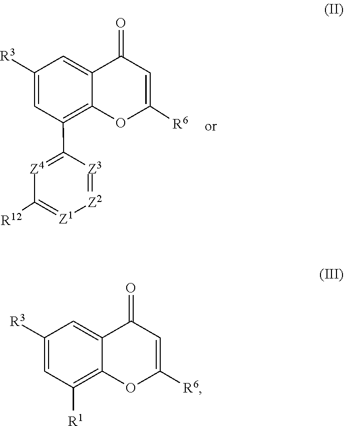
where Z¹-Z⁴ are CH or one of Z¹-Z⁴ is N and the others of Z¹-Z⁴ are CH; and
R¹² is halo, H, alkynyl, or -alkynyl-R¹¹.

6. The compound of claim 1, having a structure according to any one of formulas IIA-IIE, IIIC or IIID, or a pharmaceutically acceptable salt, hydrate, stereoisomer, or tautomer thereof: 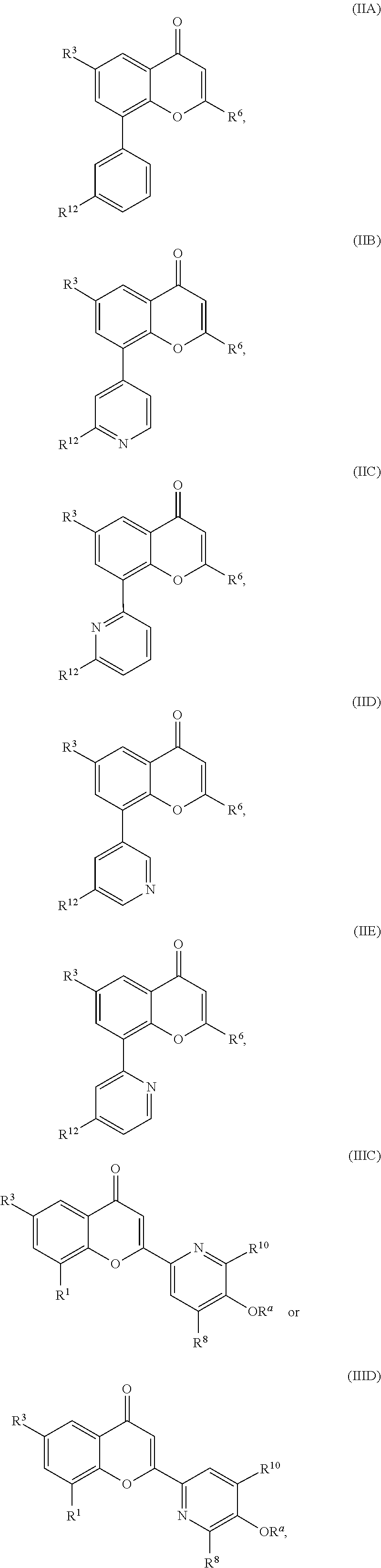
where R³ is halo or H;
R⁸ is H, substituted or unsubstituted alkyl, halo, or —OR^{a};
R¹⁰ is H, alkynyl, substituted or unsubstituted alkyl, halo, or —OR^{a};
R¹² is halo, H, or alkynyl; and
R^{a} is H, alkyl, or acyl.

7. The compound according to claim 1, wherein one of R¹, R², or R³ is -L-Q and -L-Q is: 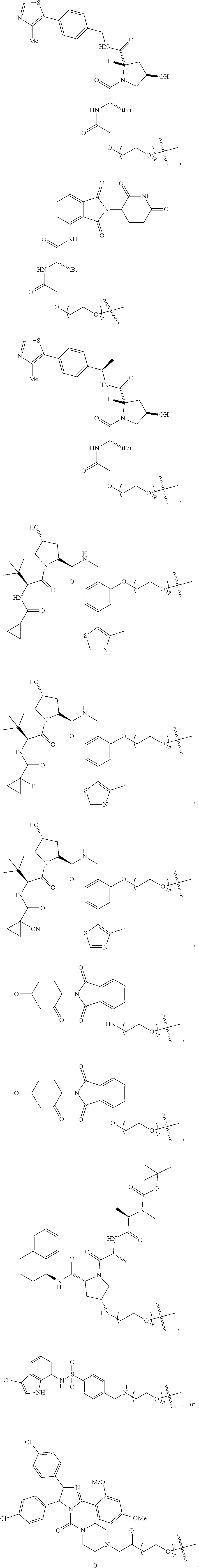
where p is an integer from 2 to 7.

8. The compound according to claim 1, wherein:
(i) the compound is capable of passing through the blood-brain barrier; or
(ii) the compound has an aqueous solubility ≥10 μM; or
(iii) the compound has an half-maximal inhibitory concentration <100 nM for CK2; or
(iv) any combination of (i), (ii), and (iii).

9. The compound of claim 1, wherein the compound is:

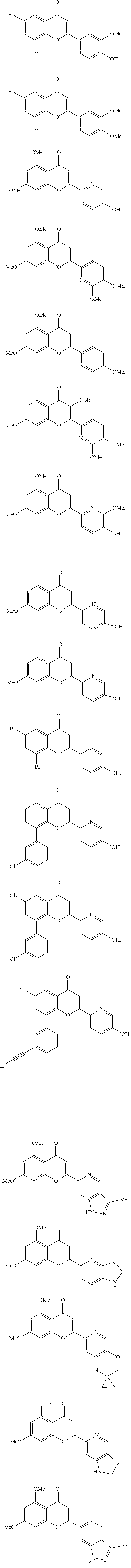

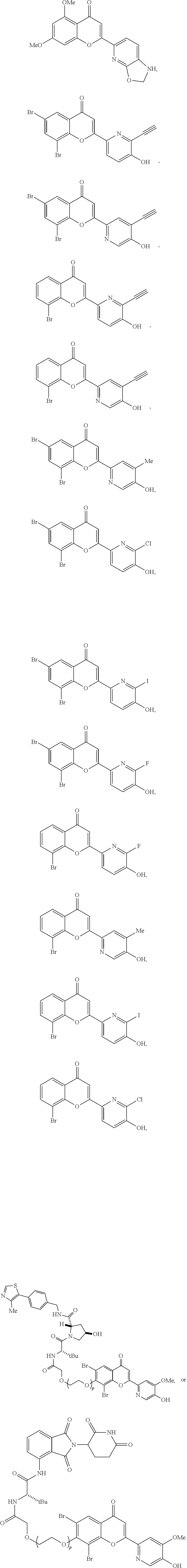

10. A pharmaceutical composition, comprising a compound according to claim 1 and a pharmaceutically acceptable excipient.

11. A method of inhibiting CK2 enzyme activity, comprising:
contacting a cell that expresses CK2 enzyme with an effective amount of one or more compounds according to claim 1, thereby inhibiting activity of the CK2 enzyme.

12. The method of claim 11, wherein the cell is an astrocyte, a microglia, a neuron, a white blood cell, an adipocyte, a myocyte, or an epithelial cell.

13. The method of claim 11, wherein inhibiting activity of the CK2 enzyme inhibits phosphorylation of one or more biomarkers, increases mitophagy, decreases mitochondrial fission, increases mitochondrial function, or any combination thereof.

14. The method of claim 13, wherein the one or more biomarkers are HMGB1, S100A9, SORCS1, IFI16, ILF2, IFNL1, ARFGAP1, RL6IP4, DTD1, SQSTM1, FERMT2, HDLBP, MAP4K4, NAV1, PNPLA6, SMC3, TMX2, IMMT, NF-KB, IκBa, FUNDC1, CK2 (pY255), or any combination thereof.

15. The method of claim 11, where contacting the cell with the one or more compounds comprises administering a therapeutically effective amount of the one or more compounds, or an amount of a pharmaceutical composition comprising the therapeutically effective amount of the one or more compounds, to a subject.

16. The method of claim 15, wherein the subject has a disease or condition characterized at least in part by dysregulated CK2 enzyme activity, and administering the therapeutically effective amount of the one or more compounds or the amount of the pharmaceutical composition comprising the therapeutically effective amount of the one or more compounds to the subject ameliorates at least one sign or symptom of the disease or condition.

17. The method of claim 16, wherein the disease or condition is characterized at least in part by inflammation.

18. The method of claim 16, wherein the disease or condition is cancer, cardiac hypertrophy, cystic fibrosis, a neurodegenerative disease, bipolar disorder, depression, a viral infection, obesity, diabetes mellitus, atherosclerosis, epilepsy, or any combination thereof.

19. The method of claim 18, wherein:
the neurodegenerative disease is Parkinson's disease, Huntington's disease, Alzheimer's disease, multiple sclerosis, or amyotrophic lateral sclerosis; or
the viral infection is a SARS-CoV-2 infection.

20. The method of claim 13, wherein R¹, R², or R³ is -L-Q, and inhibiting CK2 enzyme activity further comprises degrading the CK2 enzyme.
